(19) United States
(12) Patent Application Publication
(10) Pub. No.: US 2012/0028931 A1
(43) Pub. Date: Feb. 2, 2012

(54) HETEROCYCLIC M-GLU5 ANTAGONISTS

(75) Inventors: Amedeo Leonardi, Milan (IT); Carlo Riva, Varese (IT); Luciano Guarneri, Garbagnate Milanese (IT); Davide Graziani, Milan (IT); Fabio Marinoni, Milano (IT); Gianni Motta, Barlassina (IT); Ilaria Bettinelli, Ispra (Varese) (IT)

(73) Assignee: RECORDATI IRELAND LIMITED, Cork (IE)

(21) Appl. No.: 12/882,169

(22) Filed: Sep. 14, 2010

Related U.S. Application Data
(60) Provisional application No. 61/242,208, filed on Sep. 14, 2009.

(51) Int. Cl. A61K 31/616; A61K 31/4545; C07D 409/14; C07D 401/14; C07D 417/14; C07D 413/14; A61K 31/497; A61K 31/454; A61K 31/4439; A61K 31/55; C07D 495/04; A61K 31/4725; A61K 31/46; A61K 31/517; A61P 13/00; A61P 13/10; A61P 25/00; A61P 25/22; A61P 25/30; A61P 25/04; A61P 43/00; A61P 1/04; C07D 405/14

(52) U.S. Cl. 514/161; 546/193; 514/318; 546/194; 544/405; 514/255.05; 546/209; 514/326; 546/269.1; 514/210.2; 540/597; 514/217.04; 514/343; 514/305; 514/278; 514/252.17

(57) ABSTRACT

Compounds I

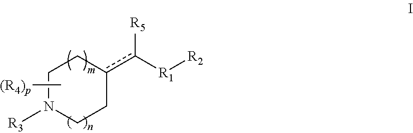

(R₁ is an optionally substituted C₁-C₁₃ heteromonocyclic, heterobicyclic or heterotricyclic group containing from 1 to 5 heteroatoms selected from N, O and S;
R₂ is H, an optionally substituted monocyclic aromatic group, or a C₁-C₅ heteroaromatic group containing from 1 to 4 heteroatoms selected from N, O and S;
R₃ is an optionally substituted C₁-C₁₃ heteromonocyclic, heterobicyclic or heterotricyclic group containing from 1 to 5 heteroatoms selected from N, O and S; an optionally substituted mono-, bi- or tricyclic C₆-C₁₄ aryl group, an optionally substituted C₃-C₆ cycloalkyl group, or an optionally substituted C₃-C₆ cycloalkenyl group;
each R₄, independently for each position capable of substitution, is H or C₁-C₆ alkyl;
R₅ is H, halogen or C₁-C₆ alkyl;
m is 0, 1 or 2; n is 0, 1 or 2; p is 0, 1, 2, 3, 4, 5, or 6; and [symbol] is an optional double bond)
   and their enantiomers, diastereomers, N-oxides and pharmaceutically acceptable salts, and pharmaceutical compositions containing them, are useful for the treatment of neuromuscular dysfunction of the lower urinary tract and also for the treatment of gastrooesophageal reflux disease; anxiety disorder; abuse, substance dependence and substance withdrawal disorders; neuropathic pain disorder, migraine and fragile X syndrome disorders.

### STATEMENT OF RELATED APPLICATIONS

This application claims priority under 35 U.S.C. §119(e) from U.S. Provisional application 61/242,208 filed on Sep. 14, 2009. The entire disclosure of this provisional application is incorporated by reference herein.

### FIELD OF THE INVENTION

This invention relates to novel heterocyclic compounds having selective affinity for the mGlu5 subtype of metabotropic receptors, to pharmaceutical compositions thereof and to uses for such compounds and compositions.

### BACKGROUND OF THE INVENTION

Lower urinary tract disorders encompass an assortment of syndromes that affect normal micturition. Lower urinary tract disorders may develop through a combination of pathological and/or age-related changes of the urogenital system, or other aetiology, e.g., neurological disorders. Individuals suffering from lower urinary tract disorders suffer from impaired quality of life, including embarrassment, poor self-perception, and a general reduction in emotional well-being, social function, and general health. Lower urinary tract disorders, moreover, may be associated with other physical ailments, including cellulites, pressure ulcers, urinary tract infections, falls with fractures, sleep deprivation, social withdrawal, depression, and sexual dysfunction. Older individuals suffering from lower urinary tract disorders may require more care from health care providers, both family and profession, which may be a factor in decisions to place them in institutions.

According to the U.S. National Institutes of Health (NIH), up to 35 million Americans are estimated to suffer lower urinary tract disorders. Lower urinary tract disorders are more common among women than men (2:1) until age 80, after which men and women are equally affected. The prevalence of lower urinary tract disorders increases with age. By the age 65, lower urinary tract disorders affect 15% to 30% of all individuals and approximately 50% of individuals in long-term care.

Agents with various modes of action have been used to treat lower urinary tract disorders. These include agents that act directly on the lower urinary tract, e.g., antimuscarinics and alpha-1 antagonists, and agents that act through the central nervous system, e.g., serotonin and/or noradrenalin reuptake inhibitors. According to the NIH, however, while some progress has been made in the diagnosis, management, and treatment of lower urinary tract disorders, these disorders frequently remain intractable. Thus, there is a continued need for improved agents, formulations and therapies to treat lower urinary tract disorders.

Glutamic acid, an excitatory amino acid, is present at synapses throughout the central nervous system and is known to act on at least two types of receptors: ionotropic and metabotropic glutamate receptors.

The principle function of ionotropic glutamate receptors is that their activation forms ligand-gated ion channels and, thereby, directly mediates electrical signalling of nerve cells, producing rapid and relatively large conductance changes in the post-synaptic membranes. Metabotropic glutamate receptors (mGluRs) regulate electrical signalling indirectly, by influencing intracellular metabolic processes via G-proteins. Changes in the post-synaptic cell that are mediated through mGluRs are consequently relatively slow over time and are not linked to rapid and large changes in neuronal membrane conductance.

Three subtypes of ionotropic glutamate receptors have been described, i.e., the NMDA, AMPA and kainate subtypes.

Eight subtypes of metabotropic glutamate receptors have been cloned. The subtypes are classified into three groups on the basis of sequence similarities, and pharmacological and biochemical properties (Spooren et al., Trends Pharmacol. Sci. 22: 331-337, 2001): Group I mGlu receptors (mGlu1 and mGlu5), Group II mGlu receptors (mGlu2 and mGlu3) and Group III mGlu receptors (mGlu4, mGlu6, mGlu7 and mGlu8).

Group I receptor mGlu5 (either human or rat) is known to comprise at least two subtypes, “a” and “b”. Subtype “b” is longer than subtype “a”, because of an alternative splicing of a 32 a.a. stretch in the C-terminal (intracellular) domain, 50 residues downstream of the beginning of the domain.

So the human mGlu5b is 1212 a.a. long, while the “a” form lacks the a. acids from 877 to 908 (n. 828 being the first of the intracellular domain). The rat mGlu5b is 1203 a.a. long, while the “a” form lacks the a. acids from 876 to 907 (n. 827 being the first of the intracellular domain). (Hermans and Challis, Biochem. J. 359: 465-484, 2001).

The mGlu receptors, belonging to family 3 of GPCRs, are characterized by two distinct topological domains: a large extracellular N-terminal domain containing a Venus fly-trap module responsible for agonist binding and the 7-TM domain plus intracellular C-terminal domain that is involved in receptor activation and G-protein coupling.

The 7-TMD of mGlu I receptors has been shown to form a binding pocket for positive and negative allosteric modulators; the negative ones have been identified thanks to high throughput screening technologies and act as non-competitive antagonists, having no effect on agonist binding. The most interesting property of these molecules, in addition to their high potency, is their remarkable subtype selectivity.

The 7-TM binding region is located in a pocket-lined by TM-III, TM-V, TM-VI and TM-VII; this site corresponds to the retinal binding pocket in rhodopsin.

Allosteric modulators of mGlu5 represent an exciting advance in demonstrating the potentiality for developing novel research tools and therapeutic agents that regulate activity of specific mGluR subtypes.

The compounds of the instant invention are reported herein as mGlu5 antagonist but actually are negative allosteric modulators acting at the 7-TM binding region.

WO 00/63166 discloses tricyclic carbamic acid derivatives useful for the treatment of different diseases, including urinary incontinence. The derivatives are disclosed to be agonists or antagonists of Group I mGlu receptors with specificity for the mGlu1 receptor.

WO 01/32632 discloses pyrimidine derivatives useful for the treatment of different diseases, including urinary incontinence. The derivatives are disclosed as selective antagonists of the mGlu1 receptor with at least 10-fold selectivity for the mGlu1 receptor over the mGlu5 receptor.

WO 01/27070 discloses new bisarylacetamides useful for the treatment of urinary incontinence, among other conditions. The molecules are disclosed to be agonists or antagonists selective for the mGlu1 receptor.

U.S. Pat. No. 6,369,222 discloses heterocycloazepinyl pyrimidine derivatives useful for the treatment of urinary incontinence, among other conditions. The derivatives are disclosed to be antagonists of the mGlu1 receptor.

The aforementioned applications and patent, therefore, disclose mGlu1 receptor antagonists as useful for treating urinary incontinence. None of the references, however, provide experimental support for treatment of urinary incontinence, either in human patients or in an animal model for lower urinary tract disease.

There is a need in the art to develop novel compounds and compositions for the treatment of lower urinary tract disorders and for the alleviation of the symptoms associated with such disorders. The present inventors have addressed this need through the development of novel heterocyclic compounds that are selective mGlu5 antagonists. The compounds of the present invention provide potent inhibition of the micturition reflex through a novel mechanism of action.

### SUMMARY OF THE INVENTION

The invention is based on the finding that selective mGlu5 antagonist compounds are useful in the treatment of lower urinary tract disorders, such as neuromuscular dysfunction of the lower urinary tract, and in the treatment of migraine and in gastroesophageal reflux disease (GERD) in mammals. mGlu5 antagonists are useful also in the treatment of anxiety disorder in mammals, and in the treatment of abuse, substance dependence and substance withdrawal disorders in mammals. Another use of mGlu5 antagonists is related to the treatment of fragile X syndrome disorders.

In one embodiment, the selective mGlu5 antagonist compounds of the present invention are used to treat a disorder of the lower urinary tract in a mammal. In this embodiment, the mGlu5 antagonist compounds of the present invention can be used to treat at least one symptom of a disorder of the lower urinary tract in a mammal.

Thus, the present invention provides a method of treating a symptom of urinary incontinence in a subject suffering from a lower urinary tract disorder, comprising administering to said subject a therapeutically effective amount of one or more of the compounds of the invention, alone or in combination with other therapeutic agents to treat urge incontinence, stress incontinence, mixed incontinence or overflow incontinence.

In certain embodiments, the compounds of the present invention are used for the treatment of a lower urinary tract disorder selected from the group consisting of overactive bladder (OAB), interstitial cystitis, prostatitis, prostadynia and benign prostatic hyperplasia (BPH). In preferred embodiments, the invention provides treatment of urinary incontinence caused by or associated with such disorders.

In another embodiment, the selective mGlu5 antagonist compounds of the present invention are used for the treatment of migraine.

In a further embodiment, the selective mGlu5 antagonist compounds of the present invention are used for the treatment of gastroesophagael reflux disease (GERD) in mammals.

In a further embodiment, the selective mGlu5 antagonist compounds of the present invention are used for the treatment of anxiety in mammals.

In a further embodiment, the selective mGlu5 antagonist compounds of the present invention are used for the treatment of abuse, substance dependence and substance withdrawal disorders in mammals.

In a further embodiment, the selective mGlu5 antagonist compounds of the present invention are used for the treatment of fragile X syndrome disorders in mammals.

In preferred embodiments, the novel selective mGlu5 antagonist compounds of the present invention are represented by Formula I

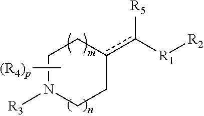

wherein:
R₁ is an optionally substituted mono-, bi- or tricyclic C₁-C₁₃ heterocyclic group containing from 1 to 5 heteroatoms selected from N, O, and S;
R₂ is hydrogen, an optionally substituted monocyclic aromatic group, or a C₁-C₅ heteroaromatic group containing from 1 to 4 heteroatoms selected from N, O, and S;
R₃ is an optionally substituted mono-, bi- or tricyclic C₁-C₁₃ heterocyclic group containing 1 to 5 heteroatoms selected from N, O, and S; an optionally substituted mono-, bi- or tricyclic C₆-C₁₄ aryl group, an optionally substituted C₃-C₆ cycloalkyl group, or an optionally substituted C₃-C₆ cycloalkenyl group;
R₄ is selected independently for each position capable of substitution from the group consisting of hydrogen and C₁-C₆ alkyl;
R₅ is hydrogen, halogen, or a C₁-C₆ alkyl group;
m is 0, 1 or 2
n is 0, 1 or 2
p is 0, 1, 2, 3, 4, 5, or 6; and
[symbol] is an optional double bond; and
   pharmaceutically acceptable salts thereof.

The optional substituents for each optionally substituted group may be selected inter alia from halogen atoms and/or from nitro, cyano, hydroxy, (C₁-C₆)-alkyl, (C₁-C₆)-alkoxy, hydroxy-(C₁-C₆)-alkyl, halo-(C₁-C₆)-alkyl and (C₁-C₆)-alkoxycarbonyl groups.

R₁ preferably represents an optionally substituted C₁-C₉ heteromonocyclic or heterobicyclic group containing from 1 to 4 heteroatoms selected from nitrogen, oxygen and sulphur.

In one alternative, R₁ represents an optionally substituted five membered C₁-C₃ heteromonocyclic group containing from 2 to 4 heteroatoms selected from nitrogen, oxygen and sulphur. Such a group may be an optionally substituted isoxazolyl, oxazolyl, oxadiazolyl, pyrazolyl, tetrazolyl, thiazolyl or triazolyl group or, more particularly, an isoxazol-3-yl, isoxazol-5-yl, 1,3-oxazol-2-yl, 1,3-oxazol-4-yl, 1,2,4-oxadiazol-3-yl, 1,2,4-oxadiazol-5-yl, 1,3,4-oxadiazol-2-yl, 3-pyrazolyl, tetrazol-2-yl, tetrazol-5-yl, 1,3-thiazol-4-yl, 1,2,3-triazol-4-yl or 1,23-triazol-5-yl group.

In another alternative, R1 represents an optionally substituted five membered C3-C4 heteromonocyclic group containing 1 or 2 heteroatoms selected from nitrogen, oxygen and sulphur, the heteromonocyclic group being fused with a benzene group. Such a group may be an optionally substituted benzimidazolyl, benzofuranyl, benzoxazolyl, benzothiazolyl, benzothienyl or indolyl group or, more particularly, a benzimidazol-2-yl, 1-benzofuran-2-yl, 1,3-benzoxazol-2-yl, 1,3-benzothiazol-2-yl, 1,3-benzothiazol-6-yl, 1-benzothien-2-yl or indol-2-yl group.

The or each optional substituent for the group R1 is preferably a halogen atom or an alkyl, alkoxy, hydroxy, hydroxyalkyl or alkoxycarbonyl group.

Most preferably, R₁ represents a 1,2,3-triazol-4-yl, 1,2,3-triazol-5-yl, 1,2,4-oxadiazol-3-yl, 3-isopropyl-1,2,4-oxadiazol-5-yl, 3-methyl-1,2,4-oxadiazol-5-yl, 1,2,4-oxadiazol-5-yl, 5-methyl-1,3,4-oxadiazol-2-yl, 1,3,4-oxadiazol-2-yl, 1,3-benzothiazol-2-yl, 1,3-benzothiazol-6-yl, 1,3-benzoxazol-2-yl, 1,3-oxazol-2-yl, 1,3-oxazol-4-yl, 2-methyl-1,3-thiazol-4-yl, 1,3-thiazol-4-yl, 4-fluoro-1-benzofuran-2-yl, 4-hydroxy-1-benzofuran-2-yl, 5-chloro-1-benzofuran-2-yl, 5-fluoro-1-benzofuran-2-yl, 5-methyl-1-benzofuran-2-yl, 6-fluoro-1-benzofuran-2-yl, 6-hydroxymethyl-1-benzofuran-2-yl, 7-fluoro-1-benzofuran-2-yl, 1-benzofuran-2-yl, 1-benzothien-2-yl, 1-methyl-3-pyrazolyl, 3-pyrazolyl, 1-methyl-benzimidazol-2-yl, benzimidazol-2-yl, 1-t.butoxycarbonyl-6-methoxy-indol-2-yl, 6-fluoro-indol-2-yl, isoxazol-3-yl, 3-methyl-isoxazol-5-yl, isoxazol-5-yl, tetrazol-2-yl, tetrazol-5-yl or thieno[3,2-b][1]benzothien-2-yl group.

R₂ preferably represents a hydrogen atom, an optionally substituted phenyl group or an optionally substituted C₁-C₅ heteroaromatic group containing from 1 to 4 heteroatoms selected from nitrogen, oxygen and sulphur. More preferably, R₂ represents a hydrogen atom, an optionally substituted phenyl group or an optionally substituted furyl, isoxazolyl, pyridyl or thienyl group.

The or each optional substituent for the group R₂ is preferably a halogen atom, an alkyl group, an alkoxy group or a trifluoromethyl group.

Most preferably, R₂ represents a hydrogen atom or a phenyl, 3-chlorophenyl, 3-fluorophenyl, 3-methoxyphenyl, 3-methylphenyl, 3-trifluoromethylphenyl, 2-furyl, 3,5-dimethyl-isoxazol-4-yl, 2-pyridyl, 6-methyl-2-pyridyl, 3-pyridyl, 4-pyridyl, 2-thienyl or 3-thienyl group.

R₃ preferably represents an optionally substituted C₂-C₉ heteromonocyclic or heterobicyclic group containing from 1 to 3 heteroatoms selected from nitrogen, oxygen and sulphur, and at least 2 adjacent carbon atoms, one of which is bonded to the nitrogen atom of the nitrogen containing ring in the general formula I. Examples of such groups are pyridyl, pyrazinyl and thienyl. The other of the said two adjacent carbon atoms is preferably substituted with an electron withdrawing group, for example, a cyano or nitro group. If further substitution is present, it is preferably by a methyl or methoxy group.

Most preferably, R₃ represents a 2-cyano-3-pyrazinyl, 3-cyano-2-thienyl, 3-cyano-4-methoxy-2-pyridyl, 3-cyano-6-methyl-2-pyridyl, 3-nitro-2-pyridyl, 6-methoxy-3-nitro-2-pyridyl or 6-methyl-3-nitro-2-pyridyl group.

R₅ preferably represents a hydrogen atom or a methyl or ethyl group.

Preferably, m=n=1.

Preferably, p=0.

Preferably, [symbol] indicates a double bond.

### DETAILED DESCRIPTION OF THE INVENTION

The inventors have tested the activity of selective mGlu1 and selective mGlu5 antagonists in a rat model that is useful to detect activity on the lower urinary tract. Surprisingly, good activity was found for antagonists selective for the mGlu5 receptor, whereas two commercially available antagonists selective for mGlu1 receptor failed to exhibit an effect. An antagonist selective for Group II mGluR receptors also failed to exhibit an effect in the rat model. Given these results, selective mGlu5 antagonists can be an effective means to treat lower urinary tract disorders.

Accordingly, the present inventors have unexpectedly found that administration of negative allosteric modulators of the glutamate mGlu5 receptor, hereinafter “mGlu5 antagonists,” provide a potent inhibition of the micturition reflex. Without wishing to be bound by any particular theory or mechanism of action, these novel compounds of present invention are thought to act in the CNS by negatively modulating the excitatory signaling to the bladder giving, as a final result, an increase of the bladder volume capacity. These modulators are thus useful for treatment of lower urinary tract disorders and symptoms thereof as described in, e.g., International Patent Application WO 04/067002 (Recordati).

### Novel Compounds of the Invention

The present invention is related to the compounds of formula I as disclosed above. The invention includes the enantiomers, diastereomers, N-oxides (e.g., piperidine N-oxides), crystalline forms, hydrates, solvates or pharmaceutically acceptable salts of the formula I compounds, as well as active metabolites of these compounds having a similar type of activity. The novel compounds of the invention are selective mGlu5 antagonists useful in the treatment of lower urinary tract disorders and for the alleviation of the symptoms associated therewith.

Except where stated otherwise, the following definitions apply throughout the present specification and claims. These definitions apply regardless of the whether a term is used by itself or in combination with other terms. Hence the definition of “alkyl” applies to “alkyl” as well as to the “alkyl” portions of “alkoxy”, “alkylamino” etc. Furthermore, all ranges described for chemical group, for example, the ranges “from 1 to 20 carbon atoms” and “C₁-C₆ alkyl” include all combinations and subcombinations of ranges and specific numbers of carbon atoms therein.

As used above, and throughout the specification, the following terms, unless otherwise indicated, shall be understood to have the following meanings:

“Alkyl” means an aliphatic hydrocarbon group, which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain, which may be straight or branched. Branched means that one or more lower alkyl groups such as methyl, ethyl or propyl are attached to a linear alkyl chain. “Lower alkyl” means an alkyl group having about 1 to about 6 carbon atoms in the chain which may be straight or branched. The term “optionally substituted alkyl” means that the alkyl group may be substituted by one or more substituents preferably 1-6 substituents, which may be the same or different, each substituent being independently selected from the groups as defined below. Non-limiting examples of suitable alkyl groups include methyl, ethyl, n-propyl, isopropyl, sec-butyl, n-butyl, and t-butyl.

“Alkenyl” means an aliphatic hydrocarbon group comprising at least one carbon-carbon double bond and which may be straight or branched and comprising about 2 to about 15 carbon atoms in the chain. Preferred alkenyl groups have about 2 to about 12 carbon atoms in the chain. More preferably about 2 to about 6 carbon atoms in the chain. Branched means that one or more lower alkyl groups such as methyl, ethyl or propyl, are attached to a linear alkenyl chain. “Lower alkenyl” means an alkenyl group having 2 to about 6 carbon atoms in the chain, which may be straight or branched. The term “optionally substituted alkenyl” means that the alkenyl group may be substituted by one or more substituents, preferably 1-6 substituents, which may be the same or different, each substituents being independently selected from the groups as defined below. Non-limiting examples of suitable alkenyl groups include ethenyl, propenyl, isopropenyl, n-butenyl, 1-hexenyl and 3-methylbut-2-enyl.

“Alkynyl” means an aliphatic hydrocarbon group comprising at least one carbon-carbon triple bond and which may be straight or branched and comprising 2 to about 15 carbon atoms in the chain. Preferred alkynyl groups have 2 to about 12 carbon atoms, particularly, 2 to about 6 carbon atoms, in the chain. Branched means that one or more lower alkyl groups such as methyl, ethyl or propyl, are attached to a linear alkynyl chain. “Lower alkynyl” means an alkynyl group having 2 to about 6 carbon atoms in the chain, which may be straight or branched. The term “optionally substituted alkynyl” means that the alkynyl group may be substituted by one or more substituents, preferably 1-6 substituents, which may be the same or different, each substituent being independently selected from the groups as defined below. Non-limiting examples of suitable alkynyl groups include ethynyl, propynyl and 2-butynyl.

“Mono-, bi- or tricyclic aryl” means an aromatic monocyclic, bicyclic or tricyclic ring system comprising 6 to 14 carbon atoms. Bi- and tricyclic aryl groups are fused at 2 or 4 points or joined at one point via a bond or a heteroatom linker (O, S, NH, or N(C₁-C₆ alkyl) (e.g., biphenyl, 1-phenylnapthyl). The aryl group can be optionally substituted on the ring with one or more substituents, preferably 1 to 6 substituents, which may be the same or different, each substituent being independently selected from the groups as defined below. Non-limiting examples of suitable aryl groups include phenyl and naphthyl. The “mono, bi or tricyclic aryl” group can also be substituted by linking two adjacent carbons on its aromatic ring via a combination of 1 to 4 carbon atoms and 1 to 3 oxygen atoms such as, for example, methylenedioxy, ethylenedioxy, and the like. Also included within the scope of the term “aryl” as it is used herein is a group in which the aryl ring is fused at two points directly or joined at one point via a bond or a heteroatom linker (O, S, NH, or N(C₁-C₆ alkyl), to one or two non aromatic carbacyclic or heterocyclic or heteroaromatic rings. Non limiting examples include indenyl, 1-phenyl-1H-imidazole, 5-phenylisoxazole, 4-phenyl-1,2,3 thiadiazole, 2-phenylpyrimidine, quinoline, 3,4-dihydro-2H-benzo[b][1,4]oxazine, benzo[d]thiazol-2(3H)-one, 1-phenylpyrrolidin-2-one, 1-phenylazetidin-2-one and the like.

“Mono-, bi-, or tricyclic heteroaromatic” means an aromatic mono-, bi, or tricyclic ring system having 1 to 14 ring carbon atoms, and containing 1-5 ring atoms chosen from N, NH, N—(CO)—C₁₋₆ alkyl, NC₁₋₆-alkyl, O, S, SO, SO₂ alone or in combination. Bi- and tricyclic aryl groups are fused at 2 or 4 points or joined at one or two points via a bond and/or a heteroatom linker (O, S, NH, or N(C₁-C₆ alkyl). The “mono, bi, or tricyclic heteroaromatic” can be optionally substituted on the ring by replacing an available hydrogen on the ring by one or more substituents which may be the same or different, each being independently selected from the groups defined below. A nitrogen atom of the mono or bicyclic heteroaromatic can be optionally oxidized to the corresponding N-oxide. Non-limiting examples of suitable heteroaromatics include furanyl, imidazolyl, isoxazolyl, oxadiazolyl, oxazolyl, pyrrolyl, pyridyl, pyrimidyl, pyridazinyl, thiazolyl, triazolyl, tetrazolyl, thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, or benzoisoxazolyl. Also included within the scope of the term “heteroaromatic”, as it is used herein, is a group in which a heteroatomic ring is fused at two points or joined at one point via a bond or a heteroatom linker (O, S, NH, or N(C₁-C₆ alkyl), to one nonaromatic, aromatic or heterocyclic rings where the radical or point of attachment is on the heteroaromatic ring. Non-limiting examples include tetrahydroquinolinyl, tetrahydroisoquinolinyl, 3-phenylpyridine, 3-cyclohexylpyridine, 3-(pyridin-3-yl)morpholine, 3-phenylisoxazole, 2-(piperidin-1-yl)pyrimidine and the like.

“Mono-, bi-, or tricyclic heterocyclic” means an aromatic or non-aromatic saturated mono bi or tricyclic ring system having 2 to 14 ring carbon atoms, and containing 1-5 ring atoms chosen from NH, N—(CO)—C₁₋₆-alkyl, NC₁₋₆-alkyl, O, SO₂ and S, alone or in combination. Bi- and tricyclic heterocyclic groups are fused at 2 or 4 points or joined at one point via a bond or a heteroatom linker (O, S, NH, or N(C₁-C₆ alkyl). The “mono bi or tricyclic heterocyclic” can be optionally substituted on the ring by replacing an available hydrogen on the ring by one or more substituents which may be the same or different, each being independently selected from the groups defined below. There are no adjacent oxygen and or sulfur atoms present in the ring system. The nitrogen or sulfur atom of the heterocyclic can be optionally oxidized to the corresponding N-oxide, S-oxide or S-dioxide. Non-limiting examples of suitable heterocyclic include furanyl, imidazolyl, isoxazolyl, oxadiazolyl, oxazolyl, pyrrolyl, pyridyl, pyrimidyl, pyridazinyl, thiazolyl, triazolyl, tetrazolyl, thienyl, carbazolyl, benzimidazolyl, benzothienyl, benzofuranyl, indolyl, quinolinyl, benzotriazolyl, benzothiazolyl, benzoxazolyl, benzimidazolyl, isoquinolinyl, isoindolyl, acridinyl, or benzoisoxazolyl. Non Limiting examples of suitable heterocyclic rings include also aziridinyl, piperidinyl, pyrrolidinyl, piperazinyl, tetrahydropyranyl, tetrahydrofuranyl, tetrahydrothiophenyl, morpholinyl, thiomorpholinyl and the like. Also included with in the scope of the term “heterocyclic” as it is used herein is a group in which the heterocyclic ring is fused at two points or joined at one point via a bond or a heteroatom linker (O, S, NH, or N(C₁-C₆ alkyl), to one aromatic, or cycloalkyl ring, non limiting examples include isoindoline-1,3-dione 1-methyl-2-phenyl-1H-pyrazole-3(2H)-one, indoline and the like.

“Mono or bicyclic cycloalkyl” means a non aromatic mono, bi or tricyclic ring system comprising 3 to about 14 carbon atoms, preferably 3-6 carbon atoms. The cycloalkyl group may optionally contain one or two double bonds within the ring (e.g., cyclohexenyl, cyclohexadiene). The cycloalkyl can be optionally substituted on the ring by replacing an available hydrogen on the ring by one or more substituents which may be the same or different, each being independently selected from the groups as defined below. Non-limiting examples of suitable monocyclic cycloalkyls include cyclopropyl, cyclopentyl, cyclohexyl, cycloheptyl and the like. Non-limiting examples of suitable multicyclic cycloalklys include 1-decalinyl, norbornyl, adamantyl and the like.

“Alkoxy” means an alkyl-O— group in which the alkyl group is as previously described. Non-limiting examples of suitable alkoxy groups include methoxy, ethoxy, n-propoxy and isopoxy. The alkyl group is linked to an adjacent moiety through the ether oxygen.

“Halogen” or “Hal” means fluorine, chlorine, bromine or iodine. Preferred are fluorine, chlorine or bromine, and more preferred are fluorine and chlorine.

“Haloalkyl” means an alkyl as defined above wherein one or more hydrogen atoms on the alkyl is replaced by a halo group defined above.

“Oxo” means a ═O moiety.

The term “alkanoyl” refers to radicals having a carbonyl radical as defined below, attached to an alkyl radical. Preferred alkanoyl radicals are “lower alkanoyl” radicals having 1-6 carbon atoms. The alkanoyl radicals may be substituted or unsubstituted, such as formyl, acetyl, propionyl (propanoyl), butanoyl (butyryl), isobutanoyl (isobutyryl), valeryl (pentanoyl), isovaleryl, pivaloyl, hexanoyl or the like.

The term “carbonyl”, whether used alone or with other terms, such as “alkylcarbonyl”, denotes —(C═O)—. The term “alkylcarbonyl” refers to radicals having a carbonyl radical substituted with an alkyl radical. More preferred alkylcarbonyl radicals are “lower alkylcarbonyl” radicals having one to six carbon atoms. Examples of such radicals include methylcarbonyl and ethylcarbonyl. The terms “alkanoyl” and “alkylcarbonyl” are synonymous.

The term “alkanoyloxy” refers to an “alkanoyl” radical as defined above linked to an oxygen radical, to generate an ester group.

The term “aminocarbonyl” when used by itself or with other terms such as “aminocarbonylalkyl”, “N-alkylaminocarbonyl”, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula —C(═O)NH₂. The terms “N-alkylaminocarbonyl” and “N,N-dialkylaminocarbonyl” denote aminocarbonyl radicals in which the amino groups have been substituted with one alkyl radical and two alkyl radicals, respectively. Preferred are “lower alkylaminocarbonyl” having lower alkyl radicals as described above attached to an aminocarbonyl radical

The term “alkylthio” refers to radicals containing a linear or branched alkyl radical, of one to ten carbon atoms, attached to a divalent sulfur atom. An example of “alkylthio” is methylthio, (CH₃—S—).

The term “amino” refers to the radical —NH₂.

The terms “N-alkylamino” and “N,N-dialkylamino” denote amino groups which have been substituted with one alkyl radical and with two alkyl radicals, respectively. More preferred alkylamino radicals are “lower alkylamino” radicals having one or two alkyl radicals of one to six carbon atoms, attached to the nitrogen atom. Examples of “alkylamino” include N-methylamino, N-ethylamino, N,N-dimethylamino, N,N-diethylamino or the like.

The term “acyl”, whether used alone, or within a term such as “acylamino”, denotes a radical provided by the residue after removal of hydroxyl from an organic acid. The term “acylamino” refers an amino radical substituted with an acyl group. An examples of an “acylamino” radical is acetylamino or acetamido (CH₃C(═O)—NH—) where the amine may be further substituted with alkyl, aryl or aralkyl.

The term “aryloxy” refers to the radical —O-aryl. Examples of such radicals include phenoxy.

The term “cyano” refers to the radical —C≡N.

The term “nitro” refers to the radical —NO₂.

The term “heterocycloalkyl” refers to the radical -Alkyl-Heterocycle.

The term “hydroxy” refers to the radical —OH.

The term “optionally substituted” means optional substitution on a specified moiety with one or more, preferably 1-8 groups, radicals or moieties which have a molecular mass of less than 300 preferably less than 200 and more preferably less than 150; independently selected for each position capable of substitution on the specified moiety.

A combination of substituents or variables is permissible only if such a combination results in a stable or chemically feasible compound. A stable compound or chemically feasible compound is one in which the chemical structure is not substantially altered when kept at temperature of about 50° C. or less, in the absence of moisture or other chemically reactive conditions, for at least 7 days.

In some embodiments, preferred compounds of Formula I are those in which R₄ is H and [symbol] is a double bond. Also preferred are compounds of Formula I in which m is 1, n is 1. Especially preferred are compounds of Formula II:

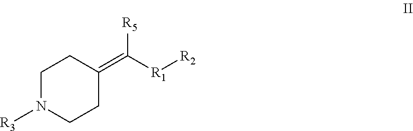

wherein the groups R₁, R₂, R₃, and R₅ are as defined above.

Preferred optional substitutents for the compounds of Formulas I and II are independently halogen, oxo, nitro, cyano, hydroxy, carbamoyl, C₁-C₆ alkylsulphonyl, C₁-C₆ alkylthio, C₁-C₆ alkylcarbonyl or C₁-C₆ alkylcarbonyl-(C₁-C₆) alkyl group, or a group of the formula —NR*R* wherein each R* independently represents hydrogen, C₁-C₆ alkyl, C₁-C₆ alkylcarbonyl, phenyl or benzyl; or

a C₁-C₆ alkyl, C₂-C₆ alkenyl, C₂-C₆ alkynyl or C₁-C₆ alkoxy group, each of which may be optionally bear from 1 to 8 substituents independently selected from oxo, halo, cyano, nitro, amino, hydroxy and phenyl; or
a C₃-C₉ mono- or bicycloalkyl group each of which may be optionally bear from 1 to 3 substituents independently selected from C₁-C₆ alkyl, oxo, halo, cyano, nitro, amino, hydroxy and phenyl substituents; or
a group of the formula -A, —O-A, —C(O)-A, —(CH₂)q-A, —NR**-A, —C(O)NR**-A, —NR**C(O)-A or —OC(O)-A, wherein
A is a phenyl group or a C₁-C₈ heterocyclic group containing from 1 to 3 heteroatoms selected from N, O, and S; each group A of being optionally substituted with from 1 to 3 groups independently selected from halo, hydroxy, cyano, nitro and C₁-C₆ alkyl;
R** is independently selected from hydrogen and C₁-C₆ alkyl, and
q is 0 or an integer from 1 to 6.

Further preferred compounds according to formulas I or II are where R₁ is an optionally substituted mono- or bicyclic or tricyclic C₁-C₁₃ heterocyclic group containing from 1 to 5 heteroatoms selected from N, O, and S.

More preferably, R₁ represents an optionally substituted mono-, bi- or tricyclic C₁-C₉ heterocyclic group containing 1 to 5 heteroatoms selected from N, O, and S. Even more preferably, R₁ is an optionally substituted five membered heteroaromatic ring containing 1 to 4 heteroatoms selected from N, O, and S. Exemplary five membered heteroaromatic ring systems include, but are not limited to, optionally substituted pyrrolyl, thienyl, furyl, pyrazolyl, imidazolyl, 1,3-oxazolyl, 1,3-thiazolyl, 1,2,4-triazolyl, 1,2,3-triazolyl, tetrazolyl, isoxazolyl, thiazolyl, isothiazolyl, 1,2,4-oxadiazolyl, 1,3,5-oxadiazolyl, 1,2,4-thiadiazolyl, 1,3,5-thiadiazolyl, benzofuryl, benzothienyl, benzo-1,3-imidazolyl, benzo-1,3-oxazolyl, and benzo-1,3-thiazolyl.

In one embodiment, R₁ is an optionally substituted group selected from pyrazolyl, benzofuranyl, triazolyl, benzothiazolyl, tetrazolyl, benzoxazolyl, thiazolyl, oxazolyl, oxadiazolyl, isoxazolyl, benzimidazolyl, benzothienyl, thienobenzothienyl, and indolyl. Preferably, the optional substituent is selected from halogen, alkyl, alkoxy, nitro, amino, hydroxyl, carboxyl, cyano, and trifluoromethyl.

Further preferred compounds according to formulas I or II are wherein R₂ is hydrogen or an optionally substituted monocyclic aromatic or C₁-C₅ heteroaromatic group containing from 1 to 4 heteroatoms selected from the group consisting of N, S, and O.

More preferably, R₂ is an optionally substituted phenyl or monocyclic C₁-C₅ heteroaromatic group containing from 1 to 4 heteroatoms selected from the group consisting of N, S, and O; particularly, R₂ is optionally substituted phenyl.

In one embodiment, R₂ is an optionally substituted group selected from phenyl, furanyl, thienyl, isoxazolyl, and pyridinyl. Preferably, the optional substituent is selected from halogen, alkyl, alkoxy, nitro, amino, hydroxyl, carboxyl, cyano, and trifluoromethyl.

Further preferred compounds according to formulas I or II are where R₃ is an optionally substituted C₁-C₆ alkyl group, an optionally substituted mono-, bi- or tricyclic C₁-C₁₃ heterocyclic group containing 1 to 5 heteroatoms selected from the group consisting of N, S, and O; an optionally substituted mono, bi or tricyclic C₆-C₁₄ aryl group, an optionally substituted C₃-C₆ cycloalkyl group, or an optionally substituted C₃-C₆ cycloalkenyl group.

More preferably, R₃ is an optionally substituted mono-, bi- or tricyclic C₁-C₁₃ heterocyclic group containing 1 to 5 heteroatoms selected from the group consisting of N, S, and O; or an optionally substituted mono, bi or tricyclic C₆-C₁₄ aryl group. Even more preferably, R₃ is a mono- or bicyclic C₁-C₉ heterocyclic group containing 1 to 3 heteroatoms selected from the group consisting of N, S, and O, and at least 2 adjacent carbon atoms.

In one embodiment, R₃ is an optionally substituted group selected from phenyl, pyridyl, pyrazinyl, and thienyl. Preferably, the optional substituent is selected from halogen, alkyl, alkoxy, nitro, amino, hydroxyl, carboxyl, cyano, and trifluoromethyl. Especially preferred amongst this group are compounds wherein R₃ is a pyridyl or phenyl group substituted with an electron withdrawing group, for example, a cyano or nitro group, and/or a methyl or methoxy group, with further substituents being optional. Most preferably, R₃ is 3-nitro-2-pyridyl, 6-methyl-3-nitro-2-pyridyl, 6-methyl-3-cyano-2-pyridyl, 4-methoxy-3-cyano-2-pyridyl, 3-cyano-2-thienyl, or 3-cyano-2-pyrazinyl.

Further preferred compounds according to formula I are where R₄ is absent and m and n are 1.

Further preferred compounds according to formulas I and II are where R₅ is hydrogen, fluorine, or methyl. Especially preferred is where R₅ is hydrogen.

In one embodiment, the compounds of the invention are compounds of formula II, wherein

R₁ is an optionally substituted group selected from pyrazolyl, benzofuranyl, triazolyl, benzothiazolyl, tetrazolyl, benzoxazolyl, thiazolyl, oxazolyl, oxadiazolyl, isoxazolyl, benzimidalolyl, benzothienyl, thienobenzothienyl, and indolyl;
R₂ is an optionally substituted group selected from phenyl, furanyl, thienyl, isoxazolyl, and pyridinyl.
R₃ is an optionally substituted group selected from phenyl, pyridyl, pyrazinyl, and thienyl; and
R₅ is hydrogen or methyl.

In a preferred embodiment for the Formula II compounds in the above paragraph, the optional substituents for R₁, R₂, and R₃ are selected from halogen, alkyl, alkoxy, nitro, amino, hydroxyl, carboxyl, cyano, and trifluoromethyl.

Further preferred are compounds selected from the group consisting of:
2-(4-{[5-(2-Furyl)-1-methyl-1H-pyrazol-3-yl]methylene}piperidin-1-yl)-6-methyl-3-nitropyridine;
6-Methyl-2-{4-[(1-methyl-5-thien-2-yl-1H-pyrazol-3-yl)methylene]piperidin-1-yl}-3-nitropyridine;
2-{4-[(5-Fluoro-1-benzofuran-2-yl)methylene]piperidin-1-yl}-3-nitropyridine;
6-Methyl-2-{4-[(5-thien-2-yl-1H-pyrazol-3-yl)methylene]piperidin-1-yl}-3-nitropyridine;
3-Nitro-2-{4-[(1-phenyl-1H-1,2,3-triazol-4-yl)methylene]piperidin-1-yl}pyridine;
2-{4-[(6-Fluoro-1-benzofuran-2-yl)methylene]piperidin-1-yl}-3-nitropyridine;
2-{[1-(6-Methyl-3-nitropyridin-2-yl)piperidin-4-ylidene]methyl}-1,3-benzothiazole;
6-Methyl-3-nitro-2-{4-[(2-phenyl-2H-tetrazol-5-yl)methylene]piperidin-1-yl}pyridine;
2-{4-[(1-Benzofuran-5-phenyl-2-yl)methylene]piperidin-1-yl}-3-nitropyridine;
2-{4-[(5-Chloro-1-benzofuran-2-yl)methylene]piperidin-1-yl}-3-nitropyridine;
2-{[1-(6-Methyl-3-nitropyridin-2-yl)piperidin-4-ylidene]methyl}-1,3-benzoxazole;
3-Nitro-2-{4-[(2-phenyl-2H-tetrazol-5-yl)methylene]piperidin-1-yl}pyridine;
6-Methyl-2-{4-[(2-methyl-1,3-thiazol-4-yl)methylene]piperidin-1-yl}-3-nitropyridine;
6-Methyl-3-nitro-2-{-4-[(1-phenyl-1H-1,2,3-triazol-4-yl)methylene]piperidin-1-yl}pyridine;
tert-Butyl 4-(3-trimethylsilylprop-2-ynylidene)piperidin-1-carboxyate;
6-Methyl-3-nitro-2-{4-[(5-phenylisoxazol-3-yl)methylene]piperidin-1-yl}pyridine;
2-{4-[1-Benzofuran-5-methyl-2-yl)methylene]piperidin-1-yl}-3-nitropyridine;
2-{4-[1-Benzofuran-2-yl)methylene]piperidin-1-yl}-3-nitropyridine;
6-Methyl-3-nitro-2-{4-[(5-phenyl-1,2,4-oxadiazol-3-yl)methylene]piperidin-1-yl}pyridine;
6-Methyl-3-nitro-2-{4-[(3-phenylisoxazol-5-yl)methylene]piperidin-1-yl}pyridine;
6-Methyl-3-nitro-2-{4-[(5-phenyl-1,3-oxazol-2-yl)methylene]piperidin-1-yl}pyridine;
6-Methyl-3-nitro-2-{4-[(5-thien-2-yl-1,3,4-oxadiazol-2-yl)methylene]piperidin-1-yl}pyridine;
6-Methyl-3-nitro-2-{4-[(3-thien-2-yl-1,2,4-oxadiazol-5-yl)methylene]piperidin-1-yl}pyridine;
6-Methyl-3-nitro-2-{4-[(1-phenyl-1H-1,2,3-triazol-5-yl)methylene]piperidin-1-yl}pyridine;
2-[4-(1-Benzofuran-2-ylmethylene)piperidin-1-yl]-6-methyl-3-nitropyridine;
6-Methyl-3-nitro-2-{4-[(5-phenyl-1,3,4-oxadiazol-2-yl)methylene]piperidin-1-yl}pyridine;
6-Methyl-3-nitro-2-{4-[(5-thien-2-ylisoxazol-3-yl)methylene]piperidin-1-yl}pyridine;
6-Methyl-3-nitro-2-{4-[(2-thien-2-yl-1,3-oxazol-4-yl)methylene]piperidin-1-yl}pyridine;
6-methyl-3-nitro-2-{4-[(5-(3-methoxyphenyl)-1,2,4-oxadiazol-3-yl)methylene]piperidin-1-yl}pyridine;
6-Methyl-3-nitro-2-{4-[(5-thien-2-yl-1,2,4-oxadiazol-3-yl)methylene]piperidin-1-yl}pyridine;
2-{[1-(6-Methyl-3-nitropyridin-2-yl)piperidin-4-ylidene]methyl}-1-benzofuran-4-ol;
2-(4-{[5-(2-Furyl)-1,2,4-oxadiazol-3-yl]methylene}piperidin-1-yl)-6-methyl-3-nitropyridine;
2-(4-{[5-(3-Thienyl)-1,2,4-oxadiazol-3-yl]methylene}piperidin-1-yl)-6-methyl-3-nitropyridine;
2-{[1-(6-Methyl-3-nitropyridin-2-yl)piperidin-4-ylidene]methyl}-1H-benzimidazole;
2-[4-(1-Benzothien-2-ylmethylene)piperidin-1-yl]-6-methyl-3-nitropyridine;
(2-{[1-(6-Methyl-3-nitropyridin-2-yl)piperidin-4-ylidene]methyl}-1-benzofuran-6-yl)methanol;
6-Methyl-3-nitro-2-[4-({5-[3-(trifluoromethyl)phenyl]-1,2,4-oxadiazol-3-yl}methylene)piperidin-1-yl]pyridine;
6-Methyl-2-{4-[(3-methyl-1,2,4-oxadiazol-5-yl)methylene]piperidin-1-yl}-3-nitropyridine;
6-{[1-(6-Methyl-3-nitropyridin-2-yl)piperidin-4-ylidene]methyl}-1,3-benzothiazole;
6-Methyl-3-nitro-2-{4-[1-(5-phenyl-1,2,4-oxadiazol-3-yl)ethylidene]piperidinyl}pyridine;
6-Methyl-2-(4-{[5-(3-methylphenyl)-1,2,4-oxadiazol-3-yl]methylene}piperidin-1-yl)-3-nitropyridine;
2-(4-{[5-(3,5-Dimethylisoxazol-4-yl)-1,2,4-oxadiazol-3-yl]methylene}piperidin-1-yl)-6-methyl-3-nitropyridine;
6-(1-Methylethyl)-2-{4-[(3-methyl-1,2,4-oxadiazol-5-yl)methylene]piperidin-1-yl}-3-nitropyridine;
3-Nitro-2-{4-[(5-phenyl-1,2,4-oxadiazol-3-yl)methylene]piperidin-1-yl}pyridine;
2-(4-{[5-(3-Chlorophenyl)-1,2,4-oxadiazol-3-yl]methylene}piperidin-1-yl)-6-methyl-3-nitropyridine;
3-{4-[(5-Phenyl-1,2,4-oxadiazol-3-yl)methylene]piperidin-1-yl}pyrazine-2-carbonitrile;
4-Methoxy-2-{4-[(5-phenyl-1,2,4-oxadiazol-3-yl)methylene]piperidin-1-yl}nicotinonitrile;
6-Methyl-3-nitro-2-{4-[(3-phenyl-1,2,4-oxadiazol-5-yl)methylene]piperidin-1-yl}pyridine;
2-{4-[(5-Phenyl-1,2,4-oxadiazol-3-yl)methylene]piperidin-1-yl}thiophene-3-carbonitrile;
1-Methyl-2-{[1-(6-methyl-3-nitropyridin-2-yl)piperidin-4-ylidene]methyl}-1H-benzimidazole;
2-[4-(1-Benzofuran-2-ylmethylene)piperidin-1-yl]-6-methoxy-3-nitropyridinebenzimidazole;
2-(4-{[5-(3-Fluorophenyl)-1,2,4-oxadiazol-3-yl]methylene}piperidin-1-yl)-6-methyl-3-nitropyridine;
6-Methyl-2-{4-[(3-methylisoxazol-5-yl)methylene]piperidin-1-yl}-3-nitropyridine;
6-Methyl-2-{4-[(5-methyl-1,3,4-oxadiazol-2-yl)methylene]piperidin-1-yl}-3-nitropyridine;
-Methyl-3-nitro-2-{4-[(5-phenyl-2H-tetrazol-2-yl)methylene]piperidin-1-yl}pyridine;
6-Methyl-2-(4-{[5-(6-methylpyridin-2-yl)-1,2,4-oxadiazol-3-yl]methylene}piperidin-1-yl)-3-nitropyridine;
3-{4-[(5-Thien-3-yl-1,2,4-oxadiazol-3-yl)methylene]piperidin-1-yl}pyrazine-2-carbonitrile;
2-(4-{[2-(2-Furyl)-1,3-thiazol-4-yl]methylene}piperidin-1-yl)-6-methyl-3-nitropyridine;
6-Methyl-3-nitro-2-{4-[(2-phenyl-1,3-thiazol-4-yl)methylene]piperidin-1-yl}pyridine;
2-{4-[(4-Fluoro-1-benzofuran-2-yl)methylene]piperidin-1-yl}-6-methyl-3-nitropyridine;
3-[4-(1-Benzofuran-2-ylmethylene)piperidin-1-yl]pyrazine-2-carbonitrile;
6-Methyl-3-nitro-2-{3-[(5-phenyl-1,2,4-oxadiazol-3-yl)methylene]azetidin-1-yl}pyridine;
(4E,Z)-1-(6-Methyl-3-nitropyridin-2-yl)-4-[(5-phenyl-1,2,4-oxadiazol-3-yl)methylene]azepane;
6-Methyl-2-(4-{[5-(3-methylphenyl)-2H-tetrazol-2-yl]methylene}piperidin-1-yl)-3-nitropyridine;
6-Methyl-3-nitro-2-{(3Z)-3-[(5-phenyl-1,2,4-oxadiazol-3-yl)methylene]pyrrolidin-1-yl}pyridine;
6-Methyl-3-nitro-2-{(3E)-3-[(5-phenyl-1,2,4-oxadiazol-3-yl)methylene]pyrrolidin-1-yl}pyridine;
6-Methyl-3-nitro-2-[4-(thieno[3,2-b][1]benzothien-2-ylmethylene)piperidin-1-yl]pyridine;
2-{4-[(7-Fluoro-1-benzofuran-2-yl)methylene]piperidin-1-yl}-6-methyl-3-nitropyridine;
tert-Butyl 6-methoxy-2-[[1-(6-methyl-3-nitro-2-pyridyl)-4-piperidylidene]methyl]indole-1-carboxylate;
6-Fluoro-2-{[1-(6-methyl-3-nitropyridin-2-yl)piperidin-4-ylidene]methyl}-1H-indole;
2-(4-{[3-(3-Chlorophenyl)-1,2,4-oxadiazol-5-yl]methylene}piperidin-1-yl)-6-methyl-3-nitropyridine;
6-Methyl-3-nitro-2-{4-[(5-pyridin-4-yl-1,2,4-oxadiazol-3-yl)methylene]piperidin-1-yl}pyridine;
6-Methyl-3-nitro-2-{4-[(5-pyridin-2-yl-1,2,4-oxadiazol-3-yl)methylene]piperidin-2-yl}pyridine;
6-Methyl-3-nitro-2-{4-[(5-pyridin-3-yl-1,2,4-oxadiazol-3-yl)methylene]piperidin-3-yl}pyridine;
2-{4-[1-(5-(3-Chlorophenyl)-1,2,4-oxadiazol-3-yl)propylidene]piperidin-1-yl}-6-methyl-3-nitropyridine
2-{4-[1-(5-(3-Chlorophenyl)-1,2,4-oxadiazol-3-yl)ethylidene]piperidin-1-yl}-6-methyl-3-nitropyridine
2-(4-{[5-(3-Chlorophenyl)-1,2,4-oxadiazol-3-yl]methylene}piperidin-1-yl)-6-methylnicotinonitrile

### Salts, Solvates, Stereoisomers, Derivatives, Prodrugs and Active Metabolites of the Compounds of the Invention

The invention also provides the enantiomers, diastereomers, N-oxides, crystalline forms, hydrates, solvates and salts of the compounds of the general formula I and general formula II, as well as prodrugs and active metabolites of these compounds having a similar type of activity.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfate, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977; 66:1).

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the federal or a state government or listed in the U.S. Pharmacopoeia or other generally recognized pharmacopeias for use in mammals, and more particularly in humans.

Typically, a pharmaceutically acceptable salt of a compound of formulas I and II may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formulas I and II and the resulting mixture evaporated to dryness (lyophilized) to obtain the acid addition salt as a solid. Alternatively, a compound of formulas I and II may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the compounds of formulas I and II may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Also included are both total and partial salts, that is to say salts with 1, 2 or 3, preferably 2, equivalents of base per mole of acid of formulas I, II or salts with 1, 2 or 3 equivalents, preferably 1 equivalent, of acid per mole of base of formulas I and II.

For the purposes of isolation or purification it is also possible to use pharmaceutically unacceptable salts. However, only the pharmaceutically acceptable, non-toxic salts are used therapeutically and they are therefore preferred.

Pharmaceutically acceptable base addition salts are formed with metals or amines, such as alkali and alkaline earth metals or organic amines Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds of the invention may have both a basic and an acidic center may and therefore be in the form of zwitterions or internal salts.

Typically, a pharmaceutically acceptable salt of a compound of formula I or II, may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formulas I or II and the resulting mixture evaporated to dryness (lyophilized) to obtain the acid addition salt as a solid. Alternatively, a compound of formula I or II may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of formula I may form solvates (e.g., hydrates) and the invention also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical solids. Marcel Decker, New York, 1999.).

The present invention also encompasses N-oxides of the compounds of formulas I and II. The term “N-oxide” means that for heterocycles containing an otherwise unsubstituted sp²N atom, the N atom may bear a covalently bound O atom, i.e., —N→O. Examples of such N-oxide substituted heterocycles include pyridyl N-oxides, pyrimidyl N-oxides, pyrazinyl N-oxides and pyrazolyl N-oxides.

Compounds of formulas I and II may have one or more chiral centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. A mixture containing unequal portions of the enantiomers is described as having an “enantiomeric excess” (ee) of either the R or S compound. The excess of one enantiomer in a mixture is often described with a % enantiomeric excess (% ee) value determined by the formula:

%ee=(R)−(S)/(R)+(S)

The ratio of enantiomers can also be defined by “optical purity” wherein the degree at which the mixture of enantiomers rotates plane polarized light is compared to the individual optically pure R and S compounds. Optical purity can be determined using the following formula:

Optical purity=enant.ₘₐⱼₒᵣ/(enant.ₘₐⱼₒᵣ+enant.ₘᵢₙₒᵣ)

The present invention encompasses all individual isomers of compounds of formulas I and II. The description or naming of a particular compound in the specification and claims is intended to include both individual enantiomers and mixtures, racemic or otherwise, thereof. Methods for the determination of stereochemistry and the resolution of stereoisomers are well-known in the art.

For many applications, it is preferred to carry out stereoselective syntheses and/or to subject the reaction product to appropriate purification steps so as to produce substantially optically pure materials. Suitable stereoselective synthetic procedures for producing optically pure materials are well known in the art, as are procedures for purifying racemic mixtures into optically pure fractions. Those of skill in the art will further recognize that invention compounds may exist in polymorphic forms wherein a compound is capable of crystallizing in different forms. Suitable methods for identifying and separating polymorphisms are known in the art.

Diastereisomers differ in both physical properties and chemical reactivity. A mixture of diastereomers can be separated into enantiomeric pairs based on solubility, fractional crystallization or chromatographic properties, e.g., thin layer chromatography, column chromatography or HPLC.

Purification of complex mixtures of diastereomers into enantiomers typically requires two steps. In a first step, the mixture of diastereomers is resolved into enantiomeric pairs, as described above. In a second step, enantiomeric pairs are further purified into compositions enriched for one or the other enantiomer or, more preferably resolved into compositions comprising pure enantiomers. Resolution of enantiomers typically requires reaction or molecular interaction with a chiral agent, e.g., solvent or column matrix. Resolution may be achieved, for example, by converting the mixture of enantiomers, e.g., a racemic mixture, into a mixture of diastereomers by reaction with a pure enantiomer of a second agent, i.e., a resolving agent. The two resulting diasteromeric products can then be separated. The separated diastereomers are then reconverted to the pure enantiomers by reversing the initial chemical transformation.

Resolution of enantiomers can also be accomplished by differences in their non-covalent binding to a chiral substance, e.g., by chromatography on homochiral adsorbants. The noncovalent binding between enantiomers and the chromatographic adsorbant establishes diastereomeric complexes, leading to differential partitioning in the mobile and bound states in the chromatographic system. The two enantiomers therefore move through the chromatographic system, e.g, column, at different rates, allowing for their separation.

Chiral resolving columns are well known in the art and are commercially available (e.g., from MetaChem Technologies Inc., a division of ANSYS Technologies, Inc., Lake Forest, Calif.). Enantiomers can be analyzed and purified using, for example, chiral stationary phases (CSPs) for HPLC. Chiral HPLC columns typically contain one form of an enantiomeric compound immobilized to the surface of a silica packing material.

D-phenylglycine and L-leucine are examples of Type I CSPs and use combinations of π-π interactions, hydrogen bonds, dipole-dipole interactions, and steric interactions to achieve chiral recognition. To be resolved on a Type I column, analyte enantiomers must contain functionality complementary to that of the CSP so that the analyte undergoes essential interactions with the CSP. The sample should preferably contain one of the following functional groups: π-acid or π-base, hydrogen bond donor and/or acceptor, or an amide dipole. Derivatization is sometimes used to add the interactive sites to those compounds lacking them. The most common derivatives involve the formation of amides from amines and carboxylic acids.

The MetaChiral ODM™ is an example of a type II CSP. The primary mechanisms for the formation of solute-CSP complexes is through attractive interactions, but inclusion complexes also play an important role. Hydrogen bonding, π-π interactions, and dipole stacking are important for chiral resolution on the MetaChiral™ ODM. Derivatization maybe necessary when the solute molecule does not contain the groups required for solute-column interactions. Derivatization, usually to benzylamides, may be required for some strongly polar molecules like amines and carboxylic acids, which would otherwise interact strongly with the stationary phase through non-specific-stereo interactions.

Compounds of formula I can be separated into diastereomeric pairs by, for example, separation by column chromatography or TLC on silica gel. These diastereomeric pairs are referred to herein as diastereomer with upper TLC Rf; and diastereomer with lower TLC Rf. The diastereomers can further be enriched for a particular enantiomer or resolved into a single enantiomer using methods well known in the art, such as those described herein.

The relative configuration of the diastereomeric pairs can be deduced by the application of theoretical models or rules (e.g. Cram's rule, the Felkin-Ahn model) or using more reliable three-dimensional models generated by computational chemistry programs. In many instances, these methods are able to predict which diasteromer is the energetically favoured product of a chemical transformation. As an alternative, the relative configuration of the diastereomeric pairs can be indirectly determined by discovering the absolute configurations of a single enantiomer in one (or both) of the diastereomeric pair(s).

The absolute configuration of the stereocenters can be determined by very well known method to those skilled in the art (e.g., X-Ray diffraction, circular dichroism). Determination of the absolute configuration can be useful also to confirm the predictability of theoretical models and can be helpful to extend the use of these models to similar molecules prepared by reactions with analogous mechanisms (e.g., ketone reductions and reductive amination of ketones by hydrides).

The present invention also encompasses stereoisomers of the syn-anti type, and mixtures thereof encountered when [symbol] is a double bond and R₄ is an alkyl group and/or m is the same and/or different from n. The stereoisomer is designated as Z (zusammen=together) if the 2 groups of highest Cahn-Ingold-Prelog priority lie on the same side of a reference plane passing through the C═C double bond and as E (entgegen=opposite) if the 2 groups of highest Cahn-Ingold-Prelog priority lie on opposite sides of above defined plane.

As noted above, the present invention also encompasses prodrugs of the compounds of formulas I and II, i.e., compounds which release an active parent drug according to formulas I and II in vivo when administered to a mammalian subject. A prodrug is a pharmacologically active or more typically an inactive compound that is converted into a pharmacologically active agent by a metabolic transformation. Prodrugs of a compound of formulas I and II are prepared by modifying functional groups present in the compound of formulas I and II in such a way that the modifications may be cleaved in vivo to release the parent compound. In vivo, a prodrug readily undergoes chemical changes under physiological conditions (e.g., are acted on by naturally occurring enzyme(s)) resulting in liberation of the pharmacologically active agent. Prodrugs include compounds of formulas I and II wherein a hydroxy, amino, or carboxy group of a formulas I and II compound is bonded to any group that may be cleaved in vivo to regenerate the free hydroxy, amino or carboxy group, respectively. Examples of prodrugs include, but are not limited to esters (e.g., acetate, formate, and benzoate derivatives) of compounds of formulas I and II or any other derivative which upon being brought to the physiological pH or through enzyme action is converted to the active parent drug. Conventional procedures for the selection and preparation of suitable prodrug derivatives are described in the art (see, for example, Bundgaard, Design of Prodrugs, Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

Unless specifically indicated, the term “active ingredient” is to be understood as referring to a compound of formulas I and II as defined herein.

Also as noted above, the present invention also encompasses active metabolites. A “metabolite” of a compound disclosed herein is a derivative of a compound which is formed when the compound is metabolised. The term “active metabolite” refers to a biologically active derivative of a compound which is formed when the compound is metabolised. The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

### Uses of the Compounds of the Invention

Another embodiment of the present invention is method of treating diseases or disorders of the lower urinary tract, including without limitation neuromuscular dysfunctions of the lower urinary tract, comprising administering to a mammal in need of such treatment an effective amount of a compound according to Formula I or II, or a pharmaceutically acceptable salt thereof.

Further preferred are where the aforementioned neuromuscular dysfunction is selected from the group consisting of urinary urgency, overactive bladder, increased urinary frequency, decreased urinary compliance (decreased bladder storage capacity), cystitis, interstitial cystitis, incontinence, urine leakage, enuresis, dysuria, urinary hesitancy and difficulty in emptying the bladder.

Another embodiment of the present invention is method of treating neuromuscular dysfunctions of the lower urinary tract comprising administering to a mammal in need of such treatment an effective amount of a compound of the invention or a pharmaceutically acceptable salt thereof, administered in combination with an antimuscarinic drug. Preferably the antimuscarinic drug is selected from the group consisting of oxybuynin, tolterodine, darifenacin, solifenacin, trospium, imidafenacin, fesoterodine and temiverine.

Another embodiment of the present invention is method of treating neuromuscular dysfunctions of the lower urinary tract comprising administering to a mammal in need of such treatment an effective amount of a compound of the invention or a pharmaceutically acceptable salt thereof, administered in combination with α1-adrenergic antagonists. Preferably the adrenergic antagonists is selected from the group consisting of prazosin, doxazosin, terazosin, alfuzosin, silodosin and tamsulosin.

Another embodiment of the present invention is method of treating neuromuscular dysfunctions of the lower urinary tract comprising administering to a mammal in need of such treatment an effective amount of a compound of the invention or a pharmaceutically acceptable salt thereof, administered in combination with a serotonin and/or noradrenalin reuptake inhibitor. Preferably the serotonin and/or noradrenalin reuptake inhibitor is selected form the group consisting of duloxetine, milnacipran, amoxapine, venlafaxine, des-venlafaxine, sibutramine, tesofensine and des-methylsibutramine.

Another embodiment of the present invention is method of treating neuromuscular dysfunctions of the lower urinary tract comprising administering to a mammal in need of such treatment an effective amount of a compound of the invention or a pharmaceutically acceptable salt thereof, administered in combination with a selective or non-selective COX inhibitor. Preferably the selective or non-selective COX inhibitor is selected from the group consisting of ibuprofen, naproxen, benoxaprofen, flurbiprofen, fenoprfen, ketoprofen, indoprofen, pirprofen, carprofen, tioxaprofe, suprofen, tiaprofenic acid, fluprofen, indomethacin, sulindac, tolmetin, zomepirac, diclofenac, fenclofenac, ibufenac, acetyl salicylic acid, piroxicam, tenoxicam, nabumetone, ketorolac, azapropazone, mefenamic acid, tolfenamic acid, diflunisal, acemetacin, fentiazac, clidanac, meclofenamic acid, flufenamic acid, niflumic acid, flufenisal, sudoxicam, etodolac, salicylic acid, benorylate, isoxicam, 2-fluoro-α-methyl[1,1′-biphenyl]-4-acetic acid 4-(nitrooxy)butyl ester, meloxicam, parecoxib and nimesulide.

Another embodiment of the present invention is a method of treating migraine comprising administering to a mammal in need of such treatment an effective amount of a compound of the invention or a pharmaceutically acceptable salt thereof.

Another embodiment of the present invention is a method of treating GERD comprising administering to a mammal in need of such treatment an effective amount of a compound of the invention or a pharmaceutically acceptable salt thereof.

Another embodiment of the present invention is a method of treating anxiety disorder comprising administering to a mammal in need of such treatment an effective amount of a compound of the invention or a pharmaceutically acceptable salt thereof.

Another embodiment of the present invention is a method of treating abuse, substance dependence and substance withdrawal disorder comprising administering to a mammal in need of such treatment an effective amount of a compound of the invention or a pharmaceutically acceptable salt thereof.

Another embodiment of the present invention is a method of treating neuropathic pain disorder comprising administering to a mammal in need of such treatment an effective amount of a compound of the invention or a pharmaceutically acceptable salt thereof.

Another embodiment of the present invention is a method of treating fragile X syndrome disorders comprising administering to a mammal in need of such treatment an effective amount of a compound of the invention or a pharmaceutically acceptable salt thereof.

The present invention also includes the enantiomers, diastereomers, N-oxides, crystalline forms, hydrates, solvates and pharmaceutically acceptable salts of the compounds of general formulas I and II, particularly those that are selective antagonists of mGlu5 receptors.

The present invention also includes metabolites of the compounds of formulae I and II that are selective mGlu5 antagonists, hereinafter referred to as active metabolites.

The present invention also contemplates prodrugs which are metabolised in the body to generate the compounds of formulae I and II that are selective mGlu5 antagonists.

In another embodiment, the present invention provides pharmaceutical compositions comprising compounds of formulae I and II that are selective mGlu5 antagonist and enantiomers, diastereomers, N-oxides, crystalline forms, hydrates, solvates or pharmaceutically acceptable salts thereof, in admixture with pharmaceutically acceptable diluents or carriers such as those disclosed.

In yet another embodiment, this invention provides a method for identifying a compound useful for treating neuromuscular dysfunction of the lower urinary tract comprising:

(a) individually measuring the binding affinity of a test compound for the mGlu5 receptor, mGlu1 receptor and Group II mGlu receptors;

(b) identifying those test compounds that:
(1) bind to a mGlu5 receptor with an affinity of at least 10⁻⁶ M, and
(2) bind to a mGlu5 receptor with an affinity at least 10-fold stronger than the affinity for the mGlu1 receptor and Group II mGlu receptors.

c) individually measuring the ability of each of the compounds identified in step (b) to act as an antagonist or inverse agonist at the mGlu5 receptor. Compounds are selected on the basis of their affinity, selectivity and specific action vis a vis the mGlu5 receptor.

Preferably, the activity of compounds identified in steps (a), (b), and (c) above is confirmed by evaluating the activity of the compound in treatment of lower urinary tract disease in humans or an animal model system. More preferably the compounds identified exhibit activity in increasing bladder volume capacity in conscious rats.

As stated above, in certain embodiments a selective mGlu5 antagonist is used to treat the aforementioned disorders by administering the antagonist in combination with known antimuscarinic drugs or serotonin and/or noradrenalin reuptake inhibitors. Analogously, a selective mGlu5 antagonist may be administered in combination with α1-adrenergic antagonists, for the therapy of lower urinary tract symptoms, whether or not these are associated with BPH.

To the same purpose, selective mGlu5 antagonists may be administered in combination with inhibitors of the enzyme cyclooxygenase (COX) which may be selective or non-selective for the COX-2 isozyme.

### Lower-Urinary Tract Disorders

The nomenclature of lower urinary tract symptoms and pathologies used herein is set forth in Abrams et al., Neurol. and Urodyn. 21:167-178 (2002) and Andersson et al., Pharmacol. Rev. 56:581-631 (2004).

Voiding dysfunctions can be roughly classified as disturbances of storage or emptying. Storage symptoms are experienced during the storage phase of the bladder, and include increased daytime frequency, nocturia (the waking at night one or more times to void), urgency (a sudden, compelling desire to pass urine that is difficult to defer), and urinary incontinence (the any involuntary leakage of urine). Urinary incontinence may be further characterized according to symptoms. Stress urinary incontinence is the involuntary leakage on effort or exertion, or on sneezing or coughing. Urge urinary incontinence is the involuntary leakage of urine accompanied by or immediately preceded by urgency. Mixed urinary incontinence is the involuntary leakage of urine associated with urgency and also with exertion, effort, sneezing or coughing. Overflow incontinence is the involuntary leakage of urine occurring after the bladder capacity has been exceeded, e.g., from a failure to empty. Enuresis also refers to any involuntary loss of urine. Nocturnal enuresis is the loss of urine occurring during sleep.

Voiding symptoms include slow stream, splitting or spraying of the urine stream, intermittent stream (intermittency, i.e., the stopping and restarting of urine flow during micturition, hesitancy (difficulty in initiating micturition resulting in a delay in the onset of voiding after the individual is ready to pass urine), straining and terminal dribble (a prolonged final part of micturition, when the flow has slowed to a trickle/dribble).

Lower urinary tract disorders may further be categorized by a constellation of symptoms (i.e., a syndrome) or by aetiology. Individuals suffering from overactive bladder (OAB) syndrome, e.g., typically suffer from symptoms of urgency, urge incontinence, increased daytime frequency or nocturia. OAB occurs as a result of detrusor muscle overactivity referred to as detrusor muscle instability. Detrusor muscle instability can arise from non-neurological abnormalities, such as bladder stones, muscle disease, urinary tract infection or drug side effects or can be idiopathic.

Neurogenic overactive bladder (or neurogenic bladder) is a type of overactive bladder which occurs as a result of detrusor muscle overactivity referred to as detrusor hyperreflexia, secondary to known neurological disorders. Patients with neurological disorders, such as stroke, Parkinson's disease, diabetes, multiple sclerosis, peripheral neuropathy, or spinal cord lesions often suffer from neurogenic overactive bladder.

Cystitis (including interstitial cystitis) is a lower urinary tract disorder of unknown aetiology that predominantly affects young and middle-aged females, although men and children can also be affected. Symptoms of interstitial cystitis can include voiding symptoms, increased daytime frequency, urgency, nocturia or suprapubic or pelvic pain related to and relieved by voiding. Many interstitial cystitis patients also experience headaches as well as gastrointestinal and skin problems. In some cases, interstitial cystitis can also be associated with ulcers or scars of the bladder.

Prostatitis and prostadynia are other lower urinary tract disorders that have been suggested to affect approximately 2-9% of the adult male population. Prostatitis is an inflammation of the prostate, and includes bacterial prostatitis (acute and chronic) and non-bacterial prostatitis. Acute and chronic bacterial prostatitis are characterized by inflammation of the prostate and bacterial infection of the prostate gland, usually associated with symptoms of pain, increased daytime frequency and/or urgency. Chronic bacterial prostatitis is distinguished from acute bacterial prostatitis based on the recurrent nature of the disorder. Chronic non-bacterial prostatitis is characterized by inflammation of the prostate which is of unknown aetiology accompanied by the presence of an excessive amount of inflammatory cells in prostatic secretions not currently associated with bacterial infection of the prostate gland, and usually associated with symptoms of pain, increased daytime frequency and/or urgency. Prostadynia is a disorder which mimics the symptoms of prostatitis absent inflammation of the prostate, bacterial infection of the prostate and elevated levels inflammatory cells in prostatic secretions. Prostadynia can be associated with symptoms of pain, increased daytime frequency and/or urgency.

Benign prostatic hyperplasia (BPH) is a non-malignant enlargement of the prostate that is very common in men over 40 years of age. BPH is thought to be due to excessive cellular growth of both glandular and stromal elements of the prostate. Symptoms of BPH can include increased frequency, urgency, urge incontinence, nocturia, and voiding symptoms, including slow stream, splitting or spraying of the urine stream, intermittency, hesitancy, straining and terminal dribble.

The present invention provides the use of an effective amount of a selective mGlu5 antagonist, for treating lower urinary tract disorders, including those described above, in a patient in need of such treatment. For example, treatment of lower urinary tract disorders includes treatment of storage symptoms or voiding symptoms. Treatment of lower urinary tract disorders also includes treatment of increased daytime frequency, nocturia, urgency, urinary incontinence, including urge incontinence, stress incontinence, mixed incontinence and overflow incontinence, enuresis, including nocturnal enuresis, slow stream, splitting or spraying of the urine stream, intermittency, hesitancy, straining and terminal dribble.

Treatment of lower urinary tract disorders also includes treatment of OAB syndrome, including treatment of one or more symptoms of urgency, urge incontinence, daytime frequency or nocturia.

Treatment of lower urinary tract disorders further encompasses treatment of any of the aforementioned conditions, symptoms and/or syndromes when caused by or associated with cystitis, including interstitial cystitis, prostatitis, BPH, neurological disorders, decreased urinary compliance (i.e., decreased bladder storage capacity). By treatment, we mean any clinically or statistically significant attenuation or amelioration of any symptom or parameter of urinary tract disorders is within the scope of the invention. Clinically significant attenuation or amelioration means perceptible to the patient and/or to the physician.

The compounds of the invention are also useful for the treatment by combination therapy of the foregoing neuromuscular dysfunctions of the lower urinary tract. The drug administered with the compound of the invention may be:
an antimuscarinic drug, such as oxybuynin, tolterodine, darifenacin, solifenacin, trospium, imidafenacin, fesoterodine or temiverine;
a serotonin and/or noradrenalin reuptake inhibitor such as duloxetine, milnacipran, amoxapine, venlafaxine, des-venlafaxine, sibutramine, tesofensine or des-methylsibutramine;
an α₁-adrenergic antagonist, such as prazosin, doxazosin, terazosin, alfuzosin, silodosin and tamsulosin; or
a selective or non-selective COX inhibitor such as ibuprofen, naproxen, benoxaprofen, flurbiprofen, fenoprofen, ketoprofen, indoprofen, pirprofen, carprofen, tioxaprofen, suprofen, tiaprofenic acid, fluprofen, indomethacin, sulindac, tolmetin, zomepirac, diclofenac, fenclofenac, ibufenac, acetyl salicylic acid, piroxicam, tenoxicam, nabumetone, ketorolac, azapropazone, mefenamic acid, tolfenamic acid, diflunisal, acemetacin, fentiazac, clidanac, meclofenamic acid, flufenamic acid, niflumic acid, flufenisal, sudoxicam, etodolac, salicylic acid, benorylate, isoxicam, 2-fluoro-α-methyl[1,1′-biphenyl]-4-acetic acid 4-(nitrooxy)butyl ester [see Wenk et al. Europ. J. Pharmacol. 453, 319-324 (2002)], meloxicam, parecoxib or nimesulide.

The compounds of the invention are also useful for the treatment of:
gastroesophageal reflux disease (GERD),
anxiety disorder,
abuse, substance dependence and substance withdrawal disorders,
neuropathic pain disorder,
migraine, and
fragile X syndrome disorders.

### Pharmaceutical Compositions Comprising a Compound of the Invention

The invention also provides pharmaceutical compositions comprising a therapeutically effective amount of a compound of formula I or II, or an enantiomer, diastereomer, N-oxide, crystalline form, hydrate, solvate, pharmaceutically acceptable salt, prodrug or active metabolite of such a compound, in admixture with a pharmaceutically acceptable excipient or diluent.

While it is possible that, for use in the methods of the invention, a compound of formula I or II may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

As noted above, a compound of formula I may be used in combination with other therapeutically active agents. Accordingly, in one aspect, the present invention provides a pharmaceutical composition comprising at least one compound of formula I or II, or a pharmaceutically acceptable derivative (e.g., a salt or solvate) thereof, and, optionally, a pharmaceutically acceptable carrier. In particular, the invention provides a pharmaceutical composition comprising a compound having the general formula I or II or an enantiomer, diastereomer, N-oxide, crystalline form, hydrate, solvate, pharmaceutically acceptable salt, prodrug or active metabolite of such a compound, in admixture with a further therapeutically effective amount of a second therapeutic agent and, optionally, a pharmaceutically acceptable carrier or diluent. The second therapeutic agent may be an antimuscarinic drug, a serotonin and/or noradrenalin reuptake inhibitor and a selective or noselective COX inhibitor.

When combined in the same formulation it will be appreciated that the two compounds must be stable and compatible with each other and the other components of the formulation. When formulated separately they may be provided in any convenient formulation, conveniently in such manner as are known for such compounds in the art.

The term “pharmaceutically acceptable” refers to molecular entities and compositions that are generally regarded as safe. In particular, pharmaceutically acceptable carriers used in the pharmaceutical compositions of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “carrier” refers to a diluent, excipient, and/or vehicle with which an active compound is administered. Carriers used in the invention may be suitable for veterinary use as well as human pharmaceutical use. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions of the invention may comprise, in addition to, the carrier any suitable binder, lubricant, suspending agent, coating agent, preservative, stabilizer, antioxidant, dye, flavouring agent(s), and/or solubilising agent.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

### Routes of Administration and Unit Dosage Forms

The routes for administration (delivery) include, but are not limited to, one or more of: oral (e.g., as a tablet, capsule, or as an ingestible solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. Therefore, the pharmaceutical compositions of the invention include those in a form especially formulated for any of those administration routes, e.g., parenteral, oral, buccal, rectal, topical, implant, ophthalmic, nasal or genito-urinary use. In preferred embodiments, the pharmaceutical compositions of the invention are formulated in a form that is suitable for oral delivery.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, the compound of formulas I and II may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the compositions may be administered in the form of tablets or lozenges, which can be formulated in a conventional manner.

Where the composition of the invention is to be administered parenterally, such administration includes one or more of: intravenously, intraarterially, intraperitoneally, intrathecally, intraventricularly, intraurethrally, intrasternally, intracranially, intramuscularly or subcutaneously administering the agent; and/or by using infusion techniques. Bolus injections are also contemplated.

Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or acsorbic acid.

For parenteral administration (human or veterinary), the compound is best used in the form of a sterile aqueous solution which may contain other substances, for example, isotonicity agents, such as a salt or glucose, to make the solution isotonic with blood. The aqueous solutions should be suitably buffered (preferably to a pH of from 3 to 9), if needed. The preparation of suitable parenteral formulations under sterile conditions is readily accomplished by standard pharmaceutical techniques well-known to those skilled in the art. of body fluids, particularly blood. Prolonged absorption of such injectable mixtures can be achieved by introduction of absorption-delaying agents, such as aluminium monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants. Sterile injectable solutions can be prepared by mixing a compound of formulas I, II with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the aldosterone receptor antagonists and desired excipients for subsequent preparation of sterile solutions.

The compounds according to the invention may be formulated for use in human or veterinary medicine by injection and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds of the invention can be administered (e.g., orally or topically) in the form of tablets, capsules, ovules, elixirs, solutions or suspensions, which may contain flavouring or colouring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents. Solid compositions such as tablets, capsules, lozenges, pastilles, pills, boluses, powder, pastes, granules, bullets or premix preparations may also be used. Solid and liquid compositions for oral use may be prepared according to methods well-known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. In addition to the new solid-state forms of pantoprazole of the present invention as the active substance, oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of suitable preservatives include sodium benzoate, ascorbic acid and esters of p-hydroxybenzoic acid.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Examples of pharmaceutically acceptable sweeteners for the oral compositions include, but are not limited to, aspartame, saccharin, saccharin sodium, sodium cyclamate, xylitol, mannitol, sorbitol, lactose and sucrose.

Examples of pharmaceutically acceptable buffers include, but are not limited to, citric acid, sodium citrate, sodium bicarbonate, dibasic sodium phosphate, magnesium oxide, calcium carbonate and magnesium hydroxide.

Examples of pharmaceutically acceptable surfactants include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons. Ointments contain the active compound suspended or dissolved in, for example, a mixture with one or more of the following mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan and oils.

Examples of useful pharmaceutically acceptable oils include but are not limited to, mineral oils, silicone oils, fatty acids, alcohols, and glycols.

Examples of suitable pharmaceutically acceptable liquid carriers include, but are not limited to, water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Examples of pharmaceutically acceptable stabilizers and antioxidants include, but are not limited to, ethylenediaminetetriacetic acid (EDTA), thiourea, tocopherol and butyl hydroxyanisole.

Examples of pharmaceutically acceptable moisturizers include, but are not limited to, glycerine, sorbitol, urea and polyethylene glycol.

Examples of pharmaceutically acceptable emollients include, but are not limited to, mineral oils, isopropyl myristate, and isopropyl palmitate.

The compounds may also be dermally or transdermally administered, for example, by use of a skin patch.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride.

As indicated, the compounds of the present invention may be administered intranasally or by inhalation and are conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT“ ”) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebuliser.

The pharmaceutical compositions of the invention may contain from 0.01 to 99% weight per volume of the active material. For topical administration, for example, the composition will generally contain from 0.01-10%, more preferably 0.01-1% of the active material.

The active agents can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage forms comprising an effective amount of the present invention may be administered to an animal, preferably a human, in need of treatment of neuromuscular dysfunction of the lower urinary tract described by E. J. McGuire in “Campbell's UROLOGY”, 5^{th} Ed 616-638, 1986, W.B. Saunders Company.

As used herein, the term “effective amount” refers to an amount that results in measurable amelioration of at least one symptom or parameter of a specific disorder. In a preferred embodiment, the compound treats disorders of the urinary tract, such as urinary urgency, overactive bladder, increased urinary frequency, reduced urinary compliance (reduced bladder storage capacity), cystitis (including interstitial cystitis), incontinence, urine leakage, enuresis, dysuria, urinary hesitancy and difficulty in emptying the bladder. In another preferred embodiment the compound treats migraine. In other preferred embodiment the compound is used to treat GERD.

In other preferred embodiment the compound is used to treat neuropathic pain.

In other preferred embodiment the compound is used to treat anxiety.

In other preferred embodiment the compound is used to treat fragile X syndrome disorders.

In other preferred embodiment the compound is used to treat substance abuse, substance dependence and substance withdrawal disorders.

The pharmaceutical composition or unit dosage form of the present invention may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the active agents of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for example, urgency and excessive frequency of urination or both, and these may be reduced using the methods of the present invention. In the case of incontinence, any reduction in the frequency or volume of unwanted passage of urine is considered a beneficial effect of the present method of treatment.

The amount of the agent to be administered can range between about 0.01 and about 25 mg/kg/day, preferably between about 0.1 and about 10 mg/kg/day and most preferably between 0.2 and about 5 mg/kg/day. It will be understood that the pharmaceutical formulations of the present invention need not necessarily contain the entire amount of the agent that is effective in treating the disorder, as such effective amounts can be reached by administration of a plurality of doses of such pharmaceutical formulations.

In a preferred embodiment of the present invention, the compounds of Formulas I and II are formulated in capsules or tablets, preferably containing 10 to 200 mg of the compounds of the invention, and are preferably administered to a patient at a total daily dose of 10 to 300 mg, more preferably 20 to 150 mg and most preferably about 50 mg, for relief of urinary incontinence and other dysfunctions.

A pharmaceutical composition for parenteral or transdermal administration may contain from about 0.01% to about 100% by weight of the active agents of the present invention, based upon 100% weight of total pharmaceutical composition.

Generally, transdermal dosage forms contain from about 0.01% to about 100% by weight of the active agents versus 100% total weight of the dosage form.

For treatment of lower urinary tract disorders, a mGlu5 antagonist may be administered in combination with at least one compound of an additional class of therapeutic agents. Such additional class could be that of antimuscarinic drugs such as, without limitation, oxybutynin, tolterodine, darifenacin, solifenacin, trospium, fesoterodine and temiverine.

Combination therapy with at least one mGlu5 antagonist may further include treatment with an alpha1-adrenergic antagonist. Preferred alpha1-adrenergic antagonists suitable for administration in combination with mGlu5 antagonists are, for example, and without limitation, prazosin, doxazosin, terazosin, alfuzosin, silodosin, and tamsulosin. Additional alpha1-adrenergic antagonists suitable for administration in combination with mGlu5antagonists are described in U.S. Pat. Nos. 5,990,114; 6,306,861; 6,365,591; 6,387,909; and 6,403,594, incorporated herein by reference in their entireties.

Combination therapy with at least one mGlu5 antagonist may further include treatment with a serotonin and/or noradrenaline reuptake inhibitor. Examples of serotonin and/or noradrenaline reuptake inhibitors include, without limitation, duloxetine, milnacipran, amoxapine, venlafaxine, des-venlafaxine, sibutramine, tesofensine and des-methylsibutramine.

In certain embodiments, a serotonin and/or noradrenalin reuptake inhibitor suitable for administration in combination with mGlu5antagonists is a selective serotonin reuptake inhibitor (i.e., an SSRI). In certain embodiments, a serotonin and/or noradrenalin reuptake inhibitors suitable for administration in combination with mGlu5antagonists is a selective noradrenalin reuptake inhibitor (i.e., an NARI).

Combination therapy with at least one mGlu5 antagonist may further include treatment with a selective or non selective COX inhibitor. Examples of COX inhibitors include, without limitations, ibuprofen, naproxen, benoxaprofen, flurbiprofen, fenoprofen, fenbufen, ketoprofen, indoprofen, pirprofen, carprofen, tioxaprofen, suprofen, tiaprofenic acid, fluprofen, indomethacin, sulindac, tolmetin, zomepirac, diclofenac, fenclofenac, ibufenac, acetyl salicylic acid, piroxicam, tenoxicam, nabumetone, ketorolac, azapropazone, mefenamic acid, tolfenamic acid, diflunisal, acemetacin, fentiazac, clidanac, meclofenamic acid, flufenamic acid, niflumic acid, flufenisal, sudoxicam, etodolac, salicylic acid, benorylate, isoxicam, 2-fluoro-α-methyl[1,1′-biphenyl]-4-acetic acid 4-(nitrooxy)butyl ester (see Wenk et al. Europ. J. Pharmacol. 453, 319-324 (2002)), meloxicam, parecoxib, nimesulide.

The pharmaceutical composition or unit dosage form may be administered in a single daily dose, or the total daily dosage may be administered in divided doses. In addition, co-administration or sequential administration of another compound for the treatment of the disorder may be desirable. To this purpose, the combined active principles are formulated into a simple dosage unit.

For combination treatment where the compounds are in separate dosage formulations, the compounds can be administered concurrently, or each can be administered at staggered intervals. For example, the compound of the invention may be administered in the morning and the antimuscarinic compound may be administered in the evening, or vice versa. Additional compounds may be administered at specific intervals too. The order of administration will depend upon a variety of factors including age, weight, sex and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

### Synthesis of the Compounds of the Invention

Compounds of the general formulas I and II and derivatives thereof may be prepared by the general methods outlined hereinafter, said methods constituting a further aspect of the invention. In the following reaction schemes and their description, the groups R₁₋₅, m, n and p have the meaning defined for the compounds of the general formulas I and II unless otherwise stated.

It will be appreciated by those skilled in the art that it may be desirable to use protected derivatives of intermediates used in the preparation of the compounds of formula I. Protection and deprotection of functional groups may be performed by methods known in the art. Hydroxy or amino groups may be protected with any hydroxy or amino protecting group (for example, as described in Greene and Wuts. Greene's Protective Groups in Organic Synthesis., Fourth Edition, John Wiley and Sons, New York, 2007). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon.

The synthesis of the target compounds is completed by removing any protecting groups which may be present in the penultimate intermediates using standard techniques, which are well-known to those skilled in the art. The deprotected final products are then purified, as necessary, using standard techniques such as silica gel chromatography, HPLC on silica gel and the like, or by recrystallization.

The compounds of the invention are generally prepared according to the following schemes:

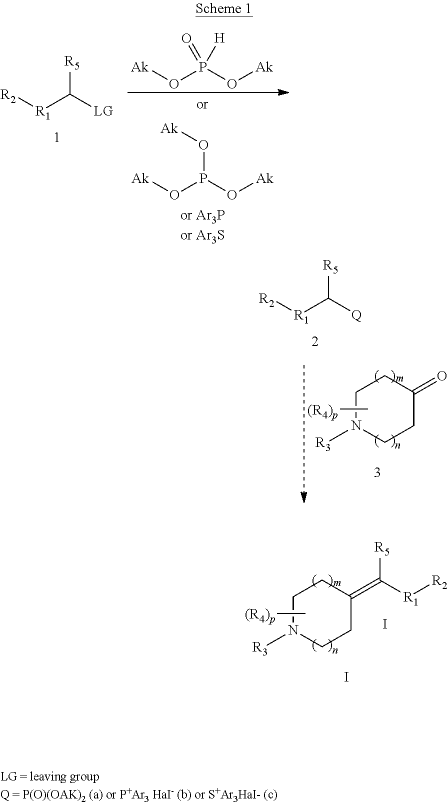

R₁₋₅, m and n are the same as given in the general formula I. Ak is lower alkyl and Z is C.

Leaving group (LG) stands for halogen, methanesulphonyloxy, p-toluenesulphonyloxy or another suitable leaving group.

Starting material (1), which is commercially available or easily prepared by standard methods known to those skilled in the art from the corresponding commercial hydroxy derivatives, is added at a temperature of −10° C. to 25° C. to a solution of the pre-formed dialkyl phosphonate anion, generated in situ from an appropriate dialkyl phospite (e.g. diethyl phosphite) upon treating with a base, preferably, but not exclusively, sodium or lithium bis-trimethylsilylamide, in an aprotic solvent, preferably THF or DME at a temperature between −50° C. and 0° C. This procedure affords compound 2a (see also Gibson, A. W.; Humphrey, G. R.; Kennedy, D. J.; Wright, S. H. B.; Synthesis 1991 (5), 414). Compounds 2a can alternatively be prepared reacting compound 1 with a trialkyl phosphite in the Michaelis-Arbuzov fashion (e.g. in triethyl phosphite at 160° C. or in a high boiling solvent (toluene, or toluene/DMF) in the presence or not of potassium iodide). Compounds 2b or 2c can be prepared by reacting 1 with a triarylphopsphine or triarylarsine in a suitable solvent (toluene, acetonitrile or the like) at a temperature ranging from −10° C. to the boiling point of the chosen solvent.

Compounds of Formula I (or II) can be obtained from 2 using standard olefination conditions such as Wittig, Horner-Emmons, or arsenic based methodologies. Some general reviews of these methodologies and directions are contained in the following references: ‘The Wittig reaction and related methods’, N. J. Lawrence in Preparation of Alkenes, J. M. J. Williams, Ed., Oxford University Press, Oxford (1996); pp 19-58; Phosphorus Ylides, O. I. Kolodiazhnyi, Wiley, N.Y. (1999); A. W. Johnson, Ylides and Imines of Phosphorus, Wiley, N.Y. (1993); Ager, D. J. Org. React. 1990, 38, 1-223.

As a usual procedure, Compound 2 is converted into the corresponding ylide by reaction with a base, preferably sodium or lithium bis-trimethylsilylamide (LiHMDS) or butyl lithium or lithium diisopropylamide (LDA) or sodium hydride or potassium tert-butoxide, in an aprotic solvent, preferably THF or DME or toluene at a temperature between −78° C. and 0° C. This ylide is then reacted with the piperidones 3 in the same reaction vessel at −60° C.-0° C. up to the boiling point of the solvent affording compounds of formula I (Gibson, A. W.; Humphrey, G. R.; Kennedy, D. J.; Wright, S. H. B.; Synthesis 1991 (5), 414 or Boehmer, J.; Schobert, R.; J Chem Res, Synop, 1998, (7), 372-373).

When the reaction is conducted using a triphenylphosphonium salt 2b, butyl lithium or LDA (lithium diisopropylamide) or LiHMDS (lithium hexamethyldisilylamide) can be preferably, but not exclusively, used to generate the phosphorus ylide in THF or other aprotic solvent (e.g. DME or THF). The ylide is then reacted with the proper piperidone 3 to provide the desired product. The phosphonate (or phosphinate or phosphine oxide) based reagents could be reacted with similar bases or with sodium or potassium methoxide or ethoxide or tert-butoxide in alcoholic solvents or with sodium hydride in aprotic solvents like dimethylsulfoxide.

Piperidones 3 are commercially available or can be easily prepared from piperidone, with the keto group free or protected as a ketal, following the procedures of, for example, acylation, (thio)carbamoylation, reductive amination, alkylation, or arylation at the basic nitrogen. All of these methodologies are well known to those skilled in the art and well documented in the literature.

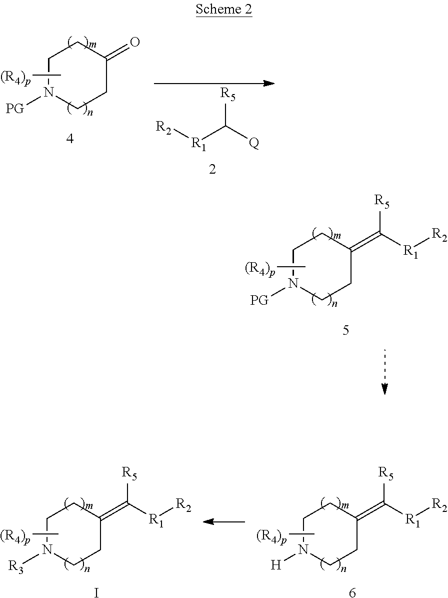

Scheme 2 represents an alternative procedure to Scheme 1 for the preparation of the compounds of formula I (and II).

R₁₋₅, m, and n are the same as given in the general formula I. PG is a protecting group and Q is as defined in Scheme 1.

The synthetic path illustrated in Scheme 2 makes use of N-protected piperidones (4; commercially available or easily prepared by standard procedures), where PG is a properly chosen amine protecting group such as, for example, tert-butoxycarbonyl (Boc), fluorenylmethoxycarbonyl (Fmoc), benzyl (Bn), benzyloxycarbonyl (Z), or trityl (Tr), arylsulphonyl. The protected piperidones 4 are reacted with compounds 2 by the same methods described in Scheme 1 for compounds 3, to afford compounds 5.

So, compounds 5 can be sequentially deprotected to 6, following standard N-deprotection procedures chosen from those reported in Greene-Wuts (Greene's Protective Groups in Organic Synthesis, 3rd Edition, Peter G. M. Wuts, Theodora W. Greene 1999, Wiley Interscience page 654-659). 6 are synthons useful for synthesizing by simple reaction procedures compounds I. These procedures embodies, for example, acylation, (thio)carbamoylation, reductive amination, alkylation, or arylation at the basic nitrogen. Arylation can be carried out by simple nucleophilic substitution (SnAr) if the aryl (heteroaryl)halogenide is enough reactive, or Pd (or Nickel) catalized reactions including the Buchwald-Hartwig amination reaction and similar methodologies. All of these methodologies are well known to those skilled in the art and well documented in the literature.

Following an additional and alternative synthetic methodology, the compounds of formula I (and II) can be prepared according to the following scheme 3:

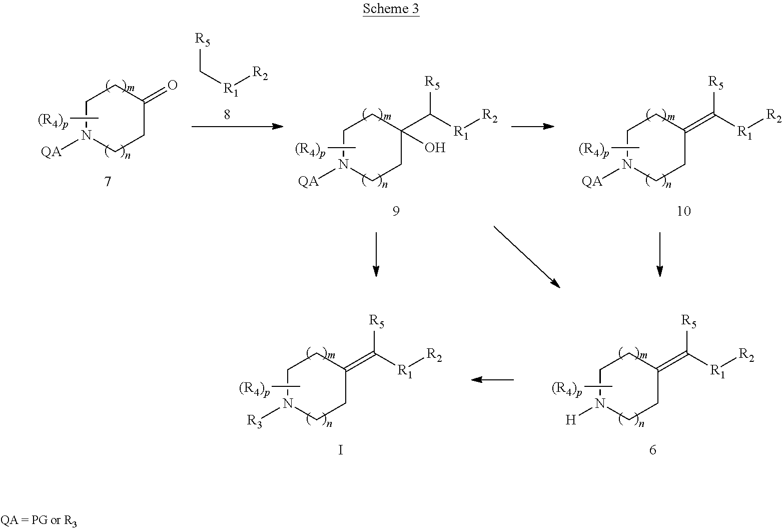

Protective group (PG) is as defined above and R₁₋₅, m, and n are the same as given in the general formula I.

By treating compound 8 with a strong base (e.g. butyl lithium, LDA, LiHMDS or the like), one can generate the carbanion at the exocyclic methyl (when R5=H) or methylene group (when R5 is not H). This carbanion is then reacted with the carbonyl compound 7 (prepared as discussed above), affording the alcohol 9. The alcohol can be reacted with strong acids (H₂SO4, CF₃COOH, CF₃SO₃H, p-tolenesulphonic acid or others) either in situ or as a crude reaction product, to afford compound 6, deprotected compound 10, or a compound of formula I directly, depending on the reaction conditions used. If compound 10 is obtained, it can be easily converted by known methodologies (see above) into compound 6. Alternative methods to generate compound 10 (or I) from 9 include the use of the Burgess reagent (Burgess reagent in organic synthesis Sachin Khapli, Satyajit Dey and Dipakranjan Mal J. Indian Inst. Sci., July-August 2001, 81, 461-476) or the conversion of the hydroxy group to a methanesulphonyloxy, p-toluenesulphonyloxy, halogen or a xanthate, followed by elimination with an appropriate base.

An additional methodology for synthesising the compounds of formula I is described in Scheme 4, in which Protective group (PG) is as defined above and R₁₋₅, m and n are the same as given in the general formula I. PR is phosphorus containing residue (e.g. dialkoxyphosphoryl, diphenoxyphosphoryl, triphenylphosphinyl, triphenylarsinyl or the like). The method outlined in Scheme 4 involves reacting compounds 7 or 12 (which are commercially available or easily prepared by standard procedures very well known in the art e.g. by simple borhydride reduction of the 4-oxo group to afford the alcohol, which can be in turn halogenated (by Mitsunobu or traditional halogenation) or converted into a tosylate, mesylate 12), following the Arbuzov displacement procedure or other suitable method, and generating phosphonium salts, phosphonates or other phosphorous (or arsenium) intermediates, which can subsequently be coupled with and aldehyde or ketone by a Wittig-Horner procedure to afford compounds 14. The preferred methodologies for carrying out this reaction sequence are discussed above for Scheme 1.

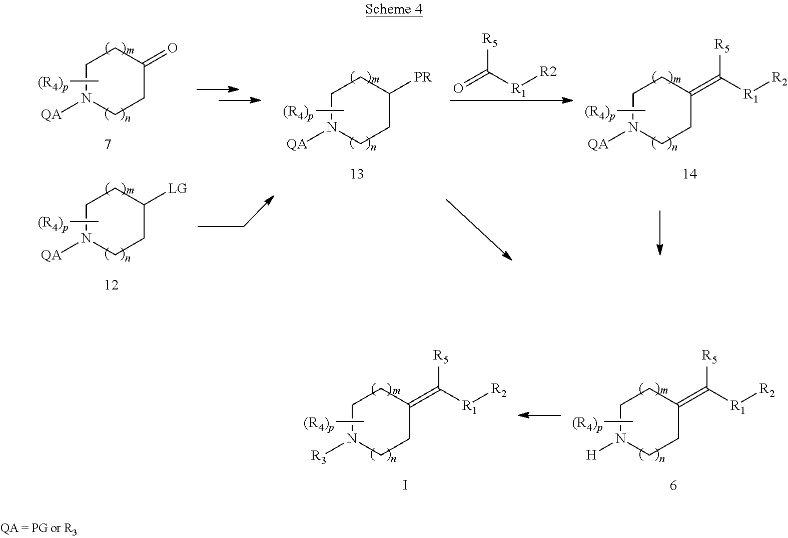

A further method for synthesising the compounds of formula I is described in Scheme 5, in which Protective group (PG) is as defined above and R₁₋₅, m and n are defined as in formula I.

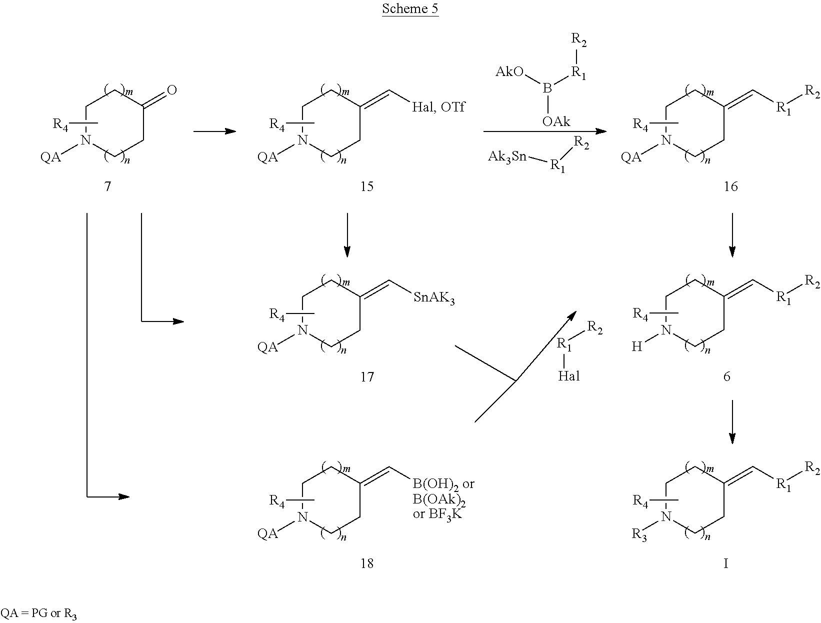

Vinyl halide compounds 15 can be obtained from 7 using standard olefination conditions such as Wittig, Horner-Emmons, Petersen or arsenic based methodologies using halomethyl phosphorus derivatives such as chloro (or bromomethyl) triphenylphosphomium chloride or diphenylchloromethylphenylphosphonate or the like. Compounds 15 can be also obtained from the corresponding exomethylene derivatives (commercially available or easily obtained from 7) carrying out first a dihalogenation of the olefinic bond (Br₂, NCS, NBS or other reagents in a suitable solvent, e.g., AcOH or a chlorinated solvent), and then dehydrohalogenation of the dihaloderivatives in the presence of a base (e.g., K₂CO₃, DBU, DMAP or the like).

Alternatively, if the same olefination reactions as above are carried out on 7 using CHBr₃ or CBr₄ or CHFBr₂ or CFBr₃ and triphenylphosphine (or another triarylphosphine optionally bonded to a polymeric resin), and optionally in the presence of a catalyst such as ZnBr₂ or diethylzinc, dihalomethylidene compounds are easily obtained. Use of CBr₄ leads to e.g. a 1,1-dibromovinyl derivative. These dihalomethilidene derivatives can be reacted with 1 equivalent of base (e.g. BuLi or NaHMDS or the like) to generate the carbanion, which is subsequently reacted with a proton source to afford 15.

The corresponding vinyl triflates 15 are obtained from the corresponding aldehydes by converting the enolate (prepared by treating the carbonyl compound with a strong base e.g. LDA at low temperature −80 to −20° C.) into the triflates using triflic anhydride, N-phenyltrifluoromethanesulphonamide or other suitable triflimide. Compound 15 can be reacted under condition suitable for, e.g., palladium catalyzed cross-coupling reactions with haloheteroaromatics or haloaromatics or with aryl or heterarylboronic acids or boron derivatives (in a Suzuki-Myauara fashion; A. Suzuki, Chem. Commun, 2005, (38), 4759-4763) or with trialkylstannyl derivatives (in a Stille fashion; March's Advanced Organic Chemistry, 6^{th} Edition, Wiley Intescience 2007, page 0792-0795). These procedures all provide compound 16. Compound 15 can also be converted into trialkylstannyl derivatives 17 through a cross coupling reaction (Han, X.; Stoltz, B. M.; Corey, E. J. J. Am. Chem. Soc. 1999, 121, 7600-7605; Gilbertson, S. R.; Challener, C. A.; Bos, M. E.; Wulff, W. D. Tetrahedron Lett. 1988, 29, 4975-4798; Wulff, W. D.; Peterson, G. A.; Bauta, W. E.; Chan, K.-S.; Faron, K. L.; Gilbertson, S. R.; Kaesler, R. W.; Yang, D. C.; Murray, C. K. J. Org. Chem. 1986, 51, 277-279) with e.g. hexamethyldistannane or by transmetallation of their lithium derivative with trimethylstannyl chloride or tributylstannyl chloride. Subsequent Stille reaction leads to compound 16.

An alternative procedure involves the preparation of intermediates 18 by cross metathesis reaction between compound 7 and a suitable vinyl boronate. Compound 18 can undergo cross-coupling reactions with haloderivatives (or triflates or tosylates or nonaflates) in the same way as compound 15.

Compounds 16, where Q is equal to PG (Protecting Group), is then deprotected to compounds 6, as described above in Scheme 2. Compound 6 can be converted to a compound of formula I using any of the standard procedures described above.

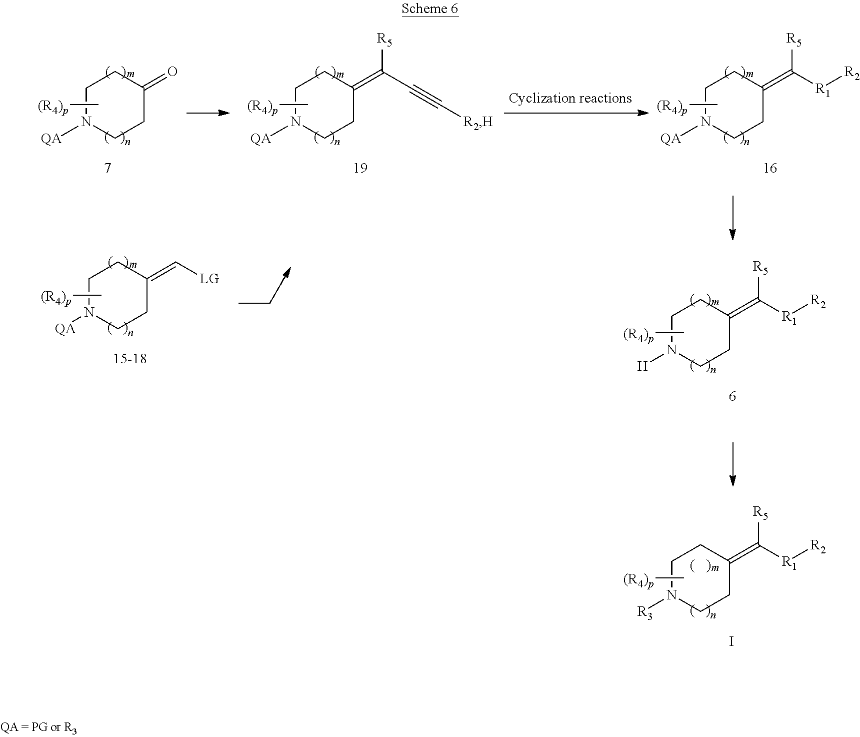

Another synthetic opportunity to afford Compound I is described in Scheme 6, in which protective group (PG) is as defined above and R₁₋₅, m and n are defined as in the general formula I.

3-(Trimethylsilyl)propargyl bromide is added at a temperature of −10° C. to 25° C. to a solution of the pre-formed phosphanion generated in situ from an appropriate dialkyl phospite (e.g. diethyl phosphite) upon treatment with a base, preferably sodium or lithium bis-trimethylsilylamide, in an aprotic solvent, preferably THF or DME at a temperature between −50° C. and 0° C. This procedure affords the compound 3-(Trimethylsilyl)propargylphosphonate (see also Gibson, A. W.; Humphrey, G. R.; Kennedy, D. J.; Wright, S. H. B.; Synthesis 1991 (5), 414). The propargylphosphonate is then converted to the corresponding stabilized ylide by reaction with a base, preferably sodium or lithium bis-trimethylsilylamide (LiHMDS), in an aprotic solvent, preferably THF or DME at a temperature between −78° C. and 0° C. This ylide is then reacted with the piperidones 7 in the same reaction vessel at −60-0° C. affording compounds 19 (R₂=Me₃Si) (Gibson, A. W.; Humphrey, G. R.; Kennedy, D. J.; Wright, S. H. B.; Synthesis 1991 (5), 414 or Boehmer, J.; Schobert, R.; J Chem Res, Synop, 1998, (7), 372-373).

The acetylenic compounds 19 can be alternatively obtained by reacting the piperidones 7 with the ylide obtained from (3-trimethylsilyl-2-propynyl)triphenylphosphonium bromide and, e.g., BuLi in THF (Hann, M. M.; Sammes, P. G.; Kennewell, P. D.; Taylor, J. B.; J Chem Soc, Perkin Trans 1, 1982, 307 or Nicolaou, K. C.; Webber, S. E.; J Am Chem Soc 1984, 106, 5734) and reacting it in a similar way as above. Another suitable procedure consists in using (3-trimethylsilyl-2-propynyl)triphenylarsonium bromide (Shen, Yanchang; Liao, Quimu; J. Organomet. Chem.; 346; 1988; 181-184) and generating the arsenic ylide with BuLi or other suitable base and then reacting it with piperidones 7.

The silyl protecting group of 19 is then removed by treatment with tetrabutylammonium fluoride (TBAF) in THF at a temperature in the range from r.t. to reflux, or by hydrolysis with base (K₂CO₃ or KOH in MeOH) or other suitable method chosen from those reported in Greene-Wuts (Greene's Protective Groups in Organic Synthesis, 3rd Edition, Peter G. M. Wuts, Theodora W. Greene 1999, Wiley Interscience page 654-659) or generally well known in the art. The acetylenic compounds 19 (where R₂=H) are then transformed into compounds 19 (R₂ not =H) by reacting them with R₂-L (where L=leaving group) following the well known Sonogashira procedure (Science of Synthesis, H. Heaney and S. Christie, October 2003, Vol. 3, Page 402 and following), that uses cuprous iodide and a palladium complex chosen from (Ph₃P)₂PdCl₂, (Ph₃P)₂Pd(OAc)₂, (Ph₃P)₄Pd (which can also be generated in situ e.g. from triphenylphosphine and Pd(OAc)₂) or any other palladium complexes cited in the literature and used for this kind of reaction, in the presence of a base such as TEA, DEA, DIPEA, TMA, butylamine, or piperidine. Solvents are chosen among THF, DME, DMF, DMA, EtOAc, DMSO, toluene and others suitable for the purpose of the reaction; or the same base in excess can be used as the reaction solvent. If one carries out the reaction in DMF or DME, the isolation of compounds 19 (R₂=H) can be avoided by adding the tetrabutylammonium fluoride or tetrabutylammonium chloride directly to the reaction medium containing 19 (R₂=Me₃Si), before the coupling (Sorensen, U.S., Pombo-Villar, E. Tetrahedron 2005, 61, 2697-2703). The R₂ substituents are introduced using aryl or heteroaryl halogenides (preferred in decreasing order iodide, bromide, chloride), aryl or heteroaryl triflates, alkyl halogenides or acyl chlorides, aroyl chlorides, heteroaroyl chlorides. Triflates are synthesized using well known methods, e.g. from phenols or hydroxyaryls (heteroaryls) using trifluoromethanesulphonic anhydride in a chlorinated solvent or using N-phenyltriflimide in toluene or a chlorinated solvent in the presence or not of a base (e.g. TEA). Both processes can be accelerated with the aid of microwaves by performing the reaction in a microwave oven. Other suitable leaving groups L for R₂-L are nonaflates, tosylates and potassium trifluoborates. Compound 19 can be obtained in a similar way (Sonogashira fashion) by reacting Compound 15-18.

Subsequent cyclization reactions (e.g. Sonogashira with ring closure or 1,3-dipolar cycloaddition without any limitation) lead to compound 16. These reactions are detailed in the experimental section that follows.

Compounds 16, where Q is equal to PG (Protecting Group), must then be deprotected to compounds 6, as described above in Scheme 2. Compounds 6 can be properly converted into Compounds I using the standard procedures described above.

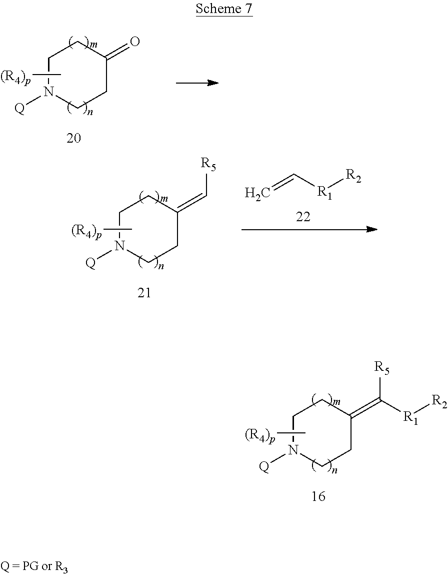

Scheme 7, describes a further possibility for synthesising the compounds of formula I. Starting from Compounds 7, Compounds 21 can be obtained using well known methods (See above for examples). A cross metathesis reaction catalyzed by. e.g., Hoveyda-Grubb's second generation ruthenium catalyst can be used to couple compounds 21 with compounds 22, affording compounds 16 (J. Am. Chem. Soc. 2000, 122. 8168). Compound 22 are commercially available or can be easily synthesised by methods well known in the art (e.g. methylenation of the corresponding aldehydes with methyltriphenylphosphonium chloride).

Compounds 16, where Q is equal to PG (Protecting Group), must then be deprotected to compounds 6, as described above in Scheme 2. Compounds 6 can be properly converted into Compounds I using the standard procedures described above.

The syntheses of other compounds not currently described in the general description above are well documented inside the experimental part of this invention which follows.

The free bases of formula I, their diastereomers or enantiomers can be converted to the corresponding pharmaceutically acceptable salts under standard conditions well known in the art. For example, the free base is dissolved in a suitable organic solvent, such as methanol, treated with, for example one equivalent of maleic or oxalic acid, one or two equivalents of hydrochloric acid or methanesulphonic acid, and then concentrated under vacuum to provide the corresponding pharmaceutically acceptable salt. The residue can then be purified by recrystallization from a suitable organic solvent or organic solvent mixture, such as methanol/diethyl ether.

The N-oxides of compounds of formula I can be synthesized by simple oxidation procedures well known to those skilled in the art.

The following examples represent synthesis of the compounds of Formula I as described generally above. These examples are illustrative only and are not intended to limit the scope of the invention. The reagents and starting materials are readily available to one of ordinary skill in the art.

As used herein, the following terms have the meanings indicated: “aq.” refers to aqueous; “eq.” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” refers to hours; “° C.” refers to degrees Celsius; “TLC” refers to thin layer chromatography; “HPLC” refers to high performance liquid chromatography; “Rf” refers to retention factor; “Rt” refers to retention time; “δ” refers to part per million down-field from tetramethylsilane; “HRMS” refers to high resolution mass spectrometry; “r.t.” refers to room temperature, “Me” refers to methyl; “Et” refers to ethyl; “Ac” refers to acetyl; “PE” refers to petroleum ether, “THF” refers to tetrahydrofuran, “CH₃CN” refers to acetonitrile, “Et₂O” refers to diethyl ether, “Ph₃P” refers to triphenylphosphine, “CBr₄” refers to tetrabromomethane, “LiHMDS” refers to lithium bis(trimethylsilyl)amide; and “LDA” refers to lithium diisopropylamide

All patents, patent applications and literature references cited in the description are hereby incorporated herein by reference in their entirety. In the case of inconsistencies, the present disclosure, including definitions, will prevail.

The present invention is not limited in scope by the specific embodiments described herein. Indeed, various modifications of the invention in addition to those described herein will become apparent to those skilled in the art from the foregoing description. Such modifications are intended to fall within the scope of the appended claims. It is further to be understood that all values given in the examples herein are approximate, and are provided for purposes of illustration.

### Example 1

### 1-(6-methyl-3-nitro-2-pyridyl)-4-{[5-(2-furyl)-1-methyl-1H-pyrazol-3-yl]-methylene}-piperidine

### 3-bromomethyl-5-(2-furyl)-1-methyl-1H-pyrazole (Compound 1a)

To a solution of 5-(2-furyl)-3-hydroxymethyl-1-methyl-1H-pyrazole (300 mg, 1.68 mmol) and PPh₃ (495 mg, 1.85 mmol) in CH₂Cl₂ (30 ml) was added CBr₄ (836 mg, 2.52 mmol) under stirring. Stirring was continued for 4 h; afterwards, the solvent was evaporated off and the crude residue was purified by automated flash chromatography (Horizon®-Biotage) eluting with PE-EtOAc 7:3 affording 405 mg of the title compound.

MS: [M+H]⁺=242.13

### Diethyl [5-(2-furyl)-1-methyl-1H-pyrazol-3-yl]-methylphosphonate (Compound 1b)

Into a solution of LiHMDS (1 M in THF, 0.622 ml, 0.622 mmol) in anhydrous THF (2.5 ml) stirred at −10° C. under nitrogen atmosphere, was dropped a solution of diethyl phosphite (0.080 ml, 0.622 mmol) in 2.5 ml of THF and the reaction mixture was kept at −10° C. per 20 min. Afterwards, was dropped Compound 1a (150 mg, 0.622 mmol) dissolved in THF (2.5 ml) and stirring continued for 2 hours at −10° C. and overnight at r.t. The reaction mixture was quenched with a saturated aqueous solution of NH₄Cl, extracted with EtOAc, dried over Na₂SO₄ and evaporated to dryness in vacuo. The crude was purified by automated flash chromatography (Horizon®-Biotage) eluting with CH₂Cl₂-MeOH 95:5 affording 186 mg of the title compound.

MS: [M+H]⁺=299.35

### 1-(6-methyl-3-nitro-2-pyridyl)-4-{[5-(2-furyl)-1-methyl-1H-pyrazol-3-yl]-methylene}-piperidine

To a solution of Compound 1b (130 mg, 0.425 mmol) in THF anhydrous (5 ml) stirred at −60° C. was added dropwise a solution of LiHMDS (1M in THF, 0.510 ml, 0.51 mmol) in THF (2 ml). Stirring was maintained at the same temperature for 15′. Afterwards, a solution of 1-(6-methyl-3-nitropyridin-2-yl)-4-oxo-piperidine (100 mg, 0.425 mmol) in 2 ml of THF was dropped and stirring was continued at −60° for 15 min; then the reaction mixture was allowed to warm up to r.t. overnight. The reaction mixture was quenched with a saturated aqueous solution of NH₄Cl, extracted with EtOAc, dried over Na₂SO₄ and evaporated to dryness in vacuo. The crude was purified by automated flash chromatography (Horizon®-Biotage) eluting with PE-EtOAc 75:25 affording 161 mg of the title compound as yellow solid.

¹H-NMR (CDCl₃, δ): 2.50 (s, 3H), 2.57-2.60 (m, 2H), 2.88-2.91 (m, 2H), 3.54-3.60 (m, 4H), 4.18 (s, 3H), 6.36 (s, 1H), 6.58 (s, 1H), 6.59-6.62 (m, 2H), 6.70 (s, 1H), 7.59 (s, 1H), 8.09-9.11 (m, 1H).

MS: [M+H]⁺=380.44

### Example 2

### 1-(6-methyl-3-nitro-2-pyridyl)-4-{[1-methyl-5-(2-thienyl)-1H-pyrazol-3-yl]-methylene}-piperidine

### 3-(Bromomethyl)-5-(2-thienyl)-1-methyl-1H-pyrazole (Compound 2a)

The title product was synthesised following the same procedure reported above for Compound 1a but using 3-hydroxymethyl-1-methyl-5-(2-thienyl)-1H-pyrazole instead of 5-(2-furyl)-3-hydroxymethyl-1-methyl-1H-pyrazole, and was obtained as colourless oil and used in the next step without further purification.

MS: [M+H]⁺=258.20

### Diethyl [1-methyl-5-(2-thienyl)-1H-pyrazol-3-yl]-methylphosphonate (Compound 2b)

To a suspension of Compound 2a (450 mg, 1.75 mmol) in toluene (15.2 ml) was added triethyl phosphite (0.3 ml, 1.75 mmol) and the reaction mixture was heated at reflux over 4 h. Dioxane (5 ml) and a further amount of triethyl phosphite (0.300 ml, 1.75 mmol) was added heating at reflux for 10 h. After evaporation, the crude residue was purified by automated flash chromatography (Horizon®-Biotage) eluting with CH₂Cl₂-MeOH 95:5 affording 550 mg of the title compound as colourless oil.

MS: [M+H]⁺=315.41

### 1-(6-methyl-3-nitro-2-pyridyl)-4-{[1-methyl-5-(2-thienyl)-1H-pyrazol-3-yl]-methylene}-piperidine

The title product was synthesised following the same procedure reported above for the compound of Example 1 but using Compound 2b instead of Compound 1b. Similar work-up and purification afforded the desired product as a yellow solid (30%).

¹H-NMR (CDCl₃, δ): 2.48 (s, 3H), 2.54-2.57 (m, 2H), 2.91-2.94 (m, 2H), 3.54-3.59 (m, 4H), 4.00 (s, 3H), 6.29 (s, 1H), 6.38 (s, 1H), 6.57-6.59 (m, 1H), 7.14-7.15 (m, 1H), 7.17 (s, 1H), 7.43-7.45 (m, 1H), 8.08-8.10 (m, 1H).

MS: [M+H]⁺=396.50

### Example 3

### 1-(3-nitro-2-pyridyl)-4-[(5-fluoro-1-benzofuran-2-yl)-methylene]-piperidine

### Diethyl (3-trimethylsilylprop-2-ynyl)-phosphonate (Compound 3a)

Into a solution of LiHMDS (1M in THF, 63.8 ml, 63.8 mmol) in anhydrous THF (162 ml) was added dropwise, under stirring at −10° C. in a nitrogen atmosphere, diethyl phosphite (7.4 ml, 63.8 mmol). The obtained solution was stirred at the same temperature for 20 min. Afterwards, 3-bromo-1-trimethylsilyl-1-propyne (10 ml, 63.8 mmol) was dropped into and the reaction mixture was stirred at −10° C. for 2 h., then quenched with water and extracted with EtOAc. The combined organic layers were washed with brine, dried on Na₂SO₄ and evaporated to dryness in vacuo to afford 14.86 g of the title product.

¹H-NMR (CDCl₃, δ): conform to Feringa, Ben L. et al., Org. Biomol. Chem., Volume 3 (14), 2005, 2524-2533

MS: [M+NH₄]⁺=266.15

### 1-(3-nitro-2-pyridyl)-4-(3-trimethysilyl-prop-2-ynylidene)-piperidine (Compound 3b)

Into a solution of Compound 1a (0.68 g, 2.74 mmol) in anhydrous THF (15 ml) stirred at −60° C. under N₂ stream, was dropped a solution of LiHMDS (1M in THF, 2.74 ml, 2.74 mmol) and the mixture was stirred at −60° C. for 15 min. To the resulting solution was added dropwise a solution of 1-(3-nitro-2-pyridyl)-4-oxo-piperidine (0.55 g, 2.49 mmol) in anhydrous THF (12 ml). The reaction mixture was stirred at −60° for 15 min., and then it was allowed to warm up to r.t. over 2 h. Afterwards, it was quenched with water and extracted with EtOAc. The combined organic layers were washed with brine, dried on Na₂SO₄ and evaporated to dryness in vacuo to afford 0.79 g of the title product. The crude was enough pure to be used in the following step without any further purification.

MS: [M+H]⁺=316.16

### 1-(3-nitro-2-pyridyl)-4-(prop-2-ynylidene)-piperidine (Compound 3c)

A solution of Compound 3b (0.57 g, 1.81 mmol), tetra-n-butylammonium fluoride hydrate (0.57 g, 2.03 mmol) in 38 ml of THF was stirred at r.t. for 2 h. The reaction mixture was poured into water and extracted with EtOAc. The combined organic layers were washed with brine, dried on Na₂SO₄ and evaporated to dryness in vacuo to afford a residue, which was purified by flash chromatography (EtOAc-Petroleum Ether 1:9) giving the title product (0.21 g).

MS: [M+H]⁺=244.13

### 1-(3-nitro-2-pyridyl)-4-[(5-fluoro-1-benzofuran-2-yl)-methylene]-piperidine

A mixture of Compound 3c (50 mg, 0.206 mmol), bis-triphenylphosphine palladium(II)dichloride (7.23 mg, 0.010 mmol), 2-bromo-4-fluorophenyl acetate (0.030 ml, 0.206 mmol), tetrabutylammonium fluoride (215 mg, 0.824) was heated at melt (80° C.) for 2 h. The reaction mixture was quenched with water and extracted with EtOAc, dried over Na₂SO₄ and evaporated to dryness in vacuo. The crude was purified by automated flash chromatography (Horizon®-Biotage) eluting with PE-EtOAc 8:2 affording 73 mg of the title compound.

¹H-NMR (CDCl₃, δ): 2.57-2.60 (m, 2H), 2.88-2.91 (m, 2H), 3.54-3.60 (m, 4H), 6.20-6.25 (s, 1H), 6.55 (s, 1H), 6.80 (m, 1H), 7.00 (m, 1H), 7.15-7.25 (d, 1H), 7.45-7.55 (m, 1H), 8.10-8.20 (d, 1H), 8.35-8.45 (m, 1H).

MS: [M+H]⁺=354.02

### Example 4

### 1-(6-methyl-3-nitro-2-pyridyl)-4-{[5-(2-thienyl)-1H-pyrazol-3-yl]-methylene}-piperidine

### 3-hydroxymethyl-1-(4-methoxybenzyl)-5-(2-thienyl)-1H-pyrazole (Compound 4a)

To a suspension of 3-hydroxymethyl-5-(2-thienyl)-1H-pyrazole (500 mg, 2.77 mmol) and K₂CO₃ (459 mg, 3.32 mmol) in CH₃CN (20 ml) was added 4-methoxybenzyl chloride (0.451 ml, 3.32 mmol) and the mixture was stirred overnight at r.t. Afterwards, it was refluxed for 4 h, evaporated to dryness and taken up with CH₂Cl₂ and H₂O, collecting and combining the organic layers, which were dried over Na₂SO₄ and evaporated to dryness in vacuo. The crude was purified by automated flash chromatography (Horizon®-Biotage) eluting with PE-EtOAc 4:6 affording 500 mg of the title compound as well as the regioisomer 5-hydroxymethyl-1-(4-methoxybenzyl)-3-(2-thienyl)-1H-pyrazole. The crude was used without further purification in the next step.

MS: [M+H]⁺=301.41

### 3-bromomethyl-1-(4-methoxybenzyl)-5-(2-thienyl)-1H-pyrazole (Compound 4b)

The title product was synthesised following the same procedure reported above for Compound 1a but using Compound 4a instead of 5-(2-furyl)-3-hydroxymethyl-1-methyl-1H-pyrazole. Purification of the crude by automated flash chromatography (Horizon®-Biotage) eluting with PE-EtOAc 8:2 afforded the wished compound (35%) as colourless oil.

MS: [M+H]⁺=365.11

### Diethyl [1-(4-methoxybenzyl)-5-(2-thienyl)-1H-pyrazol-3-yl]-methylphosphonate (Compound 4c)

The title product was synthesised following the same procedure reported above for the Compound 2b, but using Compound 4b instead of Compound 1a. Usual work-up and purification by automated flash chromatography (Horizon®-Biotage) eluting with CH₂Cl₂-MeOH 95:5 afforded the desired product as colourless oil. Yield: 89%.

MS: [M+H]⁺=421.50

### 6-Methyl-3-nitro-2-{4-[(5-thien-2-yl-1-(4-methoxybenzyl)pyrazol-3-yl)methylene]piperidin-1-yl}pyridine (Compound 4d)

The title product was synthesised following the same procedure reported above for the Compound of Example 1, but using Compound 4c instead of Compound 1b. Usual work-up and purification by automated flash chromatography (Horizon®-Biotage) eluting with PE-EtOAc 65:35 afforded the desired product as colourless oil. Yield: 28%.

MS: [M+H]⁺=502.65

### 1-(6-methyl-3-nitro-2-pyridyl)-4-{[5-(2-thienyl)-1H-pyrazol-3-yl]-methylene}-piperidine

A solution of Compound 4d (60 mg, 0.12 mmol) in trifluoroacetic acid was heated in a microwave oven (Discovery®-CEM) for 40 min at 120° C. After alkalinisation (1N NaOH), extraction with CH₂Cl₂ and evaporation in vacuo, the crude was purified by automated flash chromatography (Horizon®-Biotage) eluting with PE-EtOAc 6:4 and affording the title product as a yellow solid.

¹H-NMR (CDCl₃, δ): 2.48 (s, 3H), 2.56-2.59 (m, 2H), 2.80-2.83 (m, 2H), 3.54-3.60 (m, 4H), 6.23 (s, 1H), 6.47 (s, 1H), 6.61-6.63 (m, 1H), 7.08-7.10 (m, 1H), 7.28-7.30 (m, 1H), 7.34-7.35 (m, 1H), 8.09-8.11 (m, 1H).

MS: [M+H]⁺=382.53

### Example 5

### 1-(3-nitro-2-pyridyl)-4-[(1-phenyl-1H-1,2,3-triazol-4-yl)-methylene]-piperidine

To a solution of aniline (30 mg, 0.322 mmol) in CH₃CN (1 ml) cooled at 0-5° C., was added t-butyl nitrite (0.057 ml, 0.483 mmol) and trimethylsilyl azide (0.051 ml, 0.386 mmol). After 1 h, Compound 3c (117 mg, 0.483 mmol), cupric sulphate pentahydrate (8 mg, 0.032 mmol) and sodium ascorbate (31.9 mg, 0.161 mmol) were added. The reaction mixture was kept at r.t. overnight and then was refluxed for 8 h. After evaporation, the crude was purified by automated flash chromatography (Horizon®-Biotage) eluting with PE-EtOAc 7:3, affording the title product (117 mg) as yellow oil.

¹H-NMR (CDCl₃, δ): 2.62-2.64 (m, 2H), 3.01-3.04 (m, 2H), 3.57-3.63 (m, 4H), 6.23 (s, 1H), 6.42 (s, 1H), 6.75-6.78 (m, 1H), 7.45-7.47 (m, 1H), 7.48-7.58 (m, 2H), 7.76-7.78 (m, 2H), 7.86 (s, 1H), 8.16-8.18 (m, 1H), 8.36-8.38 (m, 1H).

MS: [M+H]⁺=363.41

### Example 6

### 1-(3-nitro-2-pyridyl)-4-[(6-fluoro-1-benzofuran-2-yl)-methylene]-piperidine

A mixture of Compound 3c (80 mg, 0.329 mmol), tetrakis(triphenylphosphine)palladium(0) (19 mg, 0.0165 mmol), 2-bromo-5-fluorophenol (0.073 ml, 0.658 mmol), Cut (9.4 mg, 0.049 mmol), butylamine (2.5 ml) and 1 ml of DMF was heated at melt at 80° C. for 10 h. After the usual work-up, the EtOAc extract was evaporated to dryness and purified by automated flash chromatography (SP1®-Biotage) eluting with PE-EtOAc gradient from 9:1 to 6:4, affording the title product (12 mg) as yellow oil.

¹H-NMR (CDCl₃, δ): 2.6 (m, 2H), 3.0-3.1 (m, 2H), 3.5-3.65 (m, 4H), 6.35 (s, 1H), 6.5 (s, 1H), 6.75-6.85 (m, 1H), 7.00 (m, 1H), 7.2 (m, 1H), 7.4-7.45 (m, 1H), 8.2 (m, 1H), 8.4 (m, 1H).

MS: [M+H]⁺=354.35

### Example 7

### 1-(6-methyl-3-nitro-2-pyridyl)-4-[(1,3-benzothiazol-2-yl)-methylene]-piperidine

### 2-(Piperidin-4-ylidenemethyl)-1,3-benzothiazole (Compound 7a)

To a solution of 2-methylbenzothiazole (0.424 ml, 3.35 mmol) in THF (15 ml) stirred at −78° C., was added LiHMDS (1M sol. in THF, 3.69 ml. 3.69 mmol). After 1 h, was dropped a solution of 1-boc-4-piperidone (701 mg, 3.52 mmol) in THF (5 ml), stirring at the same temperature for 1 h. Afterwards, the temperature was allowed to rise to r.t. and the reaction stirred overnight. Trifluoroacetic acid (2.58 ml) and toluene (15 ml) was added to the reaction mixture, heating at 90° C. for 18 h. The reaction mixture was then poured into water, alkalinized to pH 10 with 1N NaOH and extracted with EtOAc. The combined organic layers were dried over Na₂SO₄ and evaporated to dryness in vacuo. The crude (647 mg) consisting in a mixture of the title product and 2-(1,2,3,6-tetrahydropyridin-4-ylmethyl)-1,3-benzothiazole was used in the next step without further purification.

### 1-(6-methyl-3-nitro-2-pyridyl)-4-[(1,3-benzothiazol-2-yl)-methylene]-piperidine

A crude mixture of Compound 7a, 2-chloro-3-nitro-6-picoline (266 mg, 1.54 mmol) and TEA (0.293 ml, 2.1 mmol) was solubilised in N,N-dimethylacetamide (15 ml) and stirred overnight at r.t. The reaction mixture was poured into a large amount of water and extracted with EtOAc. The organic layers were washed with water, dried and evaporated to dryness in vacuo. The residue was purified by automated flash chromatography (SP1®-Biotage) eluting with a PE-Et₂O gradient from 95:5 to 5:5, affording 106 mg of the product of Example 7 and 293 mg of 4-(1,3-benzothiazol-2-ylmethyl)-6′-methyl-3′-nitro-3,6-dihydro-2H-1,2′-bipyridine.

¹H-NMR (CDCl₃, δ): 2.50 (s, 3H), 2.60-2.70 (m, 2H) 3.25-3.28 (m, 2H), 3.61-3.66 (m, 4H), 6.63 (d, 1H, J=8 Hz), 6.68 (s, 1H), 7.35-7.55 (m, 2H), 7.88 (d, 1H, J=8 Hz), 8.02 (d, 1H, J=8 Hz), 8.11 (d, 1H, J=8 Hz).

MS: [M+H]⁺=367.2

### Example 8

### 1-(6-methyl-3-nitro-2-pyridyl)-4-[(2-phenyl-2H-tetrazol-5-yl)-methylene]-piperidine

### 5-bromomethyl-2-phenyl-2H-tetrazole (Compound 8a)

The title compound was prepared as described for Compound 1a, but substituting 5-hydroxymethyl-2-phenyl-2H-tetrazole for 5-(2-furyl)-3-hydroxymethyl-1-methyl-1H-pyrazole. Usual work-up and purification by automated flash chromatography with SP1®-Biotage; (eluent: PE-EtOAc 9:1) yielded 5-(bromomethyl)-2-phenyl-2H-tetrazole (49%).

MS: [M+H]⁺=240.55

### Diethyl (2-phenyl-2H-tetrazol-5-yl)-methylphosphonate (Compound 8b)

The title compound was prepared as described for Compound 2b but substituting Compound 8a for Compound 2a. Usual work-up and purification (automated flash chromatography by Horizon®-Biotage; eluent: EtOAc-MeOH 95:5) yielded diethyl (2-phenyl-2H-tetrazol-5-yl)-methylphosphonate (93%).

MS: [M+H]⁺=297.28

### 1-(6-methyl-3-nitro-2-pyridyl)-4-[(2-phenyl-2H-tetrazol-5-yl)-methylene]-piperidine

The title product was synthesized following the same procedure reported above for the Compound of Example 1, but using Compound 8b instead of Compound 1b. Usual work-up and purification (automated flash chromatography (Horizon®-Biotage) eluting with PE-EtOAc 8:2) afforded the desired product as a yellow solid. Yield: 32.4%.

¹H-NMR (CDCl₃, δ): 2.51 (s, 3H), 2.67-2.68 (m, 2H), 3.25-3.26 (m, 2H), 3.61-3.63 (m, 4H), 6.55 (s, 1H), 6.62-6.64 (m, 1H), 7.49-7.51 (m, 1H), 7.53-7.60 (m, 2H), 8.11-8.17 (m, 3H).

MS: [M+H]⁺=378.52

### Example 9

### 1-(3-nitro-2-pyridyl)-4-[(5-phenyl-1-benzofuran-2-yl)-methylene]-piperidine

A mixture of Compound 3c (60 mg, 0.25 mmol), 2-iodo-4-phenylphenol (95 mg, 0.321 mmol), CuI (4.7 mg, 0.025 mg), bis(triphenylphosphine)palladium(II)dichloride (8.68 mg, 0.012 mmol) and 3.5 ml of TEA was stirred at 80° C. for 2 h. The reaction mixture was poured into water and extracted with EtOAc. The organic layers were washed with water, dried (Na₂SO₄) and evaporated to dryness in vacuo. Purification by automated flash chromatography (Horizon®-Biotage) eluting with PE-EtOAc 9:1 afforded 26 mg of the title product.

¹H-NMR (CDCl₃, δ): 2.6 (m, 2H), 3.0-3.1 (m, 2H), 3.5-3.65 (m, 4H), 6.35 (s, 1H), 6.5 (s, 1H), 6.75-6.85 (m, 1H), 7.00 (m, 1H), 7.2 (m, 1H), 7.4-7.45 (m, 1H), 8.2 (m, 1H), 8.4 (m, 1H).

MS: [M+H]⁺=412.35

### Example 10

### 1-(3-nitro-2-pyridyl)-4-[(5-chloro-1-benzofuran-2-yl)-methylene]-piperidine

The title compound was synthesized following exactly the same procedure reported above for the compound of Example 9 but using 4-chloro-2-iodophenol instead of 2-iodo-4-phenylphenol. Yield: 15%.

¹H-NMR (CDCl₃, δ): 2.6 (m, 2H), 3.0-3.1 (m, 2H), 3.5-3.65 (m, 4H), 6.35 (s, 1H), 6.5 (s, 1H), 6.85 (m, 1H), 7.00 (m, 1H), 7.2 (m, 1H), 7.4 (m, 1H), 7.5 (m, 1H), 8.2 (m, 1H), 8.4 (m, 1H)

MS: [M+H]⁺=370.80

### Example 11

### 1-(6-methyl-3-nitro-2-pyridyl)-4-[(1,3-benzoxazol-2-yl)-methylene]-piperidine

### 1-(t-butoxycarbonyl)-4-(1,3-benzoxazol-2-ylmethyl)-4-hydroxy-piperidine (Compound 11a)

The title compound was prepared following the procedure described above for compound 7a but using 2-methyl-1,3-benzoxazole instead of 2-methyl-1,3-benzothiazole and avoiding the addition of trifluoroacetic acid but carrying out the reaction work-up. Purification by automated flash chromatography by SP1®-Biotage (eluent: PE-EtOAc gradient from 9:1 to 4:6) afforded the title compound Yield: 30%.

¹H-NMR (CDCl₃, δ): 2.51 (s, 3H), 2.60-2.70 (m, 2H), 3.30-3.40 (m, 2H), 3.60-3.70 (m, 4H), 6.39 (s, 1H), 6.64 (d, J=8 Hz, 1H,), 7.30-7.40 (m, 2H), 7.50-7.60 (m, 1H), 7.70-7.78 (m, 1H) 8.12 (d, J=8 Hz, 1H,).

MS: [M+H]⁺=333.19

### 2-(piperidin-4-ylidenemethyl)-1,3-benzoxazole (Compound 11b)

A solution of Compound 11a (430 mg, 1.29 mmol), trifluoroacetic acid (0.99 ml, 12.9 mmol) in 25 ml of toluene was refluxed for 16 h. 98% H₂SO₄ (2 ml) was added, distilling off 20 ml of the solvent and integrating it with 20 ml of fresh toluene. After 18 h, the reaction mixture was poured into water, alkalinised with 1N NaOH and extracted with EtOAc. The combined extracts were washed with water, dried (Na₂SO₄) and evaporated to dryness in vacuo. Purification by automated flash chromatography (Horizon®-Biotage) eluting with CHCl₃—NH₃ sol. in MeOH 100:5 afforded 266 mg of the title product.

MS: [M+H]⁺=215.1

### 1-(6-methyl-3-nitro-2-pyridyl)-4-[(1,3-benzoxazol-2-yl)-methylene]-piperidine

The title compound was obtained as reported for the compound of Example 7 but starting from Compound 11b instead of Compound 7a. Yellow oil. Yield: 70%.

¹H-NMR (CDCl₃, δ): 2.51 (s, 3H), 2.60-2.70 (m, 2H), 3.30-3.40 (m, 2H), 3.60-3.70 (m, 4H), 6.39 (s, 1H), 6.64 (d, J=8 Hz, 1H,), 7.30-7.40 (m, 2H), 7.50-7.60 (m, 1H), 7.70-7.78 (m, 1H) 8.12 (d, J=8 Hz, 1H,)

MS: [M+H]⁺=351.2

### Example 12

### 1-(3-nitro-2-pyridyl)-4-[(2-phenyl-2H-tetrazol-5-yl)-methylene]-piperidine

Following the method reported for the compound of Example 8 but using 1-(3-nitro-2-pyridyl)-4-oxo-piperidine instead of 1-(6-methyl-3-nitro-2-pyridyl)-4-oxo-piperidine the title compound was obtained as a yellow solid. Yield: 35%.

¹H-NMR (CDCl₃, δ): 2.67-2.70 (m, 2H), 3.25-3.28 (m, 2H), 3.61-3.66 (m, 4H), 6.56 (s, 1H), 6.78-6.81 (m, 1H), 7.49-7.51 (m, 1H), 7.53-7.60 (m, 2H), 8.14-8.20 (m, 3H), 8.38-8.39 (m, 1H).

MS: [M+H]⁺=364.40

### Example 13

### 1-(6-methyl-3-nitro-2-pyridyl)-4-[(2-methyl-1,3-thiazol-4-yl)-methylene]-piperidine

### Diethyl (2-methyl-1,3-thiazol-4-yl)-methylphosphonate (Compound 13a)

4-Chloromethyl-2-methyl-1,3-thiazole (350 mg, 2.37 mmol) and triethyl phosphite (1 ml) were heated at 120° C. for 2 days. Afterwards, the reaction mixture was evaporated in vacuo and the residue purified by automated flash chromatography (Horizon®-Biotage) eluting with CH₂Cl₂-MeOH 95:5 and yielding the title product. Yield: 56%.

MS: [M+H]⁺=250.31

### 1-(6-methyl-3-nitro-2-pyridyl)-4-[(2-methyl-1,3-thiazol-4-yl)-methylene]-piperidine

The title product was synthesized following the same procedure reported above for the Compound of Example 1, but using Compound 13a instead of Compound 1b. Usual work-up and purification by automated flash chromatography (Horizon®-Biotage) eluting with PE-EtOAc 8:2) afforded the desired product as a yellow solid. Yield: 20%.

¹H-NMR (CDCl₃, δ): 2.48 (s, 3H), 2.53-2.56 (m, 2H), 2.75 (s, 3H), 3.00-3.02 (m, 2H), 3.52-3.59 (m, 4H), 6.37 (s, 1H), 6.57-6.59 (m, 1H), 6.89 (s, 1H), 8.08-8.10 (m, 1H).

MS: [M+H]⁺=331.52

### Example 14

### 1-(6-methyl-3-nitro-2-pyridyl)-4-[(1-phenyl-1H-1,2,3-triazol-4-yl)-methylene]-piperidine

### 1-(t-butoxycarbonyl)-4-(3-trimethylsilylprop-2-ynylidene)-piperidine (Compound 14a)

The title compound was obtained following the procedure described for Compound 3b but using 1-(t-butoxycarbonyl)-4-oxo-piperidine instead of 1-(3-nitro-2-pyridyl)-4-oxo-piperidine. After the usual work-up procedure, evaporation of the combined EtOAc extracts afforded a crude which was enough pure to be used in the next step without further purification.

¹H-NMR (CDCl₃, 6): 0.21 (s, 9H), 1.50 (s, 9H), 2.21-2.27 (m, 2H), 2.48-2.53 (m, 2H), 3.40-3.51 (m, 4H), 5.40 (s, 1H)

MS: [M+H]⁺=294.29

### 1-(t-butoxycarbonyl)-4-(prop-2-ynylidene)-piperidine (Compound 14a)

The title compound was obtained following the procedure described for Compound 3c, but using Compound 14a instead of Compound 3b. After the usual work-up procedure, evaporation of the combined EtOAc extracts afforded a crude, which was enough pure to be used in the next step without further purification.

¹H-NMR (CDCl₃, 6): 1.50 (s, 9H), 2.20-2.27 (m, 2H), 2.48-2.53 (m, 2H), 3.02 (s, 1H), 3.40-3.51 (m, 4H), 5.48 (s, 1H)

MS: [M+H]⁺=222.23

### 1-(t-butoxycarbonyl)-4-[(1-phenyl-1H-1,2,3-triazol-4-yl)-methylene]-piperidine (Compound 14c)

The title product was synthesised following the procedure described for the compound of Example 5 but substituting Compound 14b for Compound 3c. Purification of the crude, obtained by the usual work-up, was done by automated flash chromatography (Horizon®-Biotage) eluting with PE-EtOAc 7:3 and afforded the desired product as yellow oil. Yield: 32%.

MS: [M+H]⁺=341.43

### 4-[(1-phenyl-1H-1,2,3-triazol-4-yl)-methylene]-piperidine (Compound 14d)

A solution of Compound 14c (70 mg, 0.206 mmol) and trifluoroacetic acid (0.174 ml, 2.26 mmol) in 5 ml of chloroform was stirred overnight at r.t. After alkalinization with 1N NaOH and extraction with dichloromethane, the combined extracts were dried over Na₂SO₄ and evaporated to dryness in vacuo. The resultant colourless oil was used in the next step without further purification.

MS: [M+H]⁺=241.32

### 1-(6-methyl-3-nitro-2-pyridyl)-4-[(1-phenyl-1H-1,2,3-triazol-4-yl)-methylene]-piperidine

The title compound was prepared following the method described for the compound of Example 7 but using Compound 14d instead of Compound 7a. Purification of the crude obtained from the usual work-up was done by automated flash chromatography (Horizon®-Biotage) eluting with PE-EtOAc 9:1) and afforded the desired product as a yellow solid. Yield: 67%.

¹H-NMR (CDCl₃, δ): 2.50 (s, 3H), 2.60-2.63 (m, 2H), 2.99-3.02 (m, 2H), 3.57-3.62 (m, 4H), 6.41 (s, 1H), 6.60-6.62 (m, 1H), 7.45-7.47 (m, 1H), 7.48-7.58 (m, 2H), 7.76-7.78 (m, 2H), 7.86 (s, 1H), 8.16-8.18 (m, 1H).

MS: [M+H]⁺=377.43

### Example 15

### 1-(6-methyl-3-nitro-2-pyridyl)-4-[(5-phenyl-isoxazol-3-yl)-methylene]-piperidine

### Diethyl 4-hydroxy-2-oxo-4-phenylbut-3-enylphosphonate (Compound 15a)

Into a suspension of NaH 60% mineral oil dispersion (227 mg, 5.67 mmol) in 15 ml of anhydrous THF anhydrous was dropped a solution of diethyl 2-oxopropylphosphonate (1 g, 5.15 mmol) in 15 ml of THF and the resulting reaction mixture was stirred at r.t. for 1 h. Afterwards, it was cooled to 0° C. and LDA (2 M in THF, n-heptane, ethylbenzene, 5.15 ml, 10.3 mmol) was dropped, stirring at the same temperature for 30 min. After cooling to −60° C., ethyl benzoate (0.814 ml, 5.67 mmol) solubilised in 5 ml of THF was added. The reaction mixture was allowed to warm up to r.t. over 2 hours, quenched with water and 1 N HCl and extracted with Et₂O, which was dried (Na₂SO₄) and evaporated to dryness in vacuo. The crude was purified by automated flash chromatography (Horizon®-Biotage) eluting with CH₂Cl₂-MeOH 98:2 affording 1.54 g of the title product as brownish oil.

MS: [M+H]⁺=299.28

### Diethyl (5-phenylisoxazol-3-yl)-lmethylphosphonate (Compound 15b)

A solution of compound 15a (300 mg, 1.01 mmol) and hydroxylamine HCl (700 mg, 10.1 mmol) in EtOH (20 ml) was refluxed for 8 h, cooled to r.t., diluted with 1N NaOH and water, extracted with CH₂Cl₂, dried (Na₂SO₄) and evaporated to dryness in vacuo. The crude was purified by automated flash chromatography (Horizon®-Biotage) eluting with CH₂Cl₂-MeOH 98:2 affording 298 mg of the title product as brownish oil, containing about 16% (¹H-NMR determination) of the corresponding isomer diethyl 3-(phenylisoxazol-5-yl)-methylphosphonate, but used in the next step without further purification.

MS: [M+H]⁺=296.29

### 1-(6-methyl-3-nitro-2-pyridyl)-4-[(5-phenyl-isoxazol-3-yl)-methylene]-piperidine

The title compound was prepared following the methodology reported for the compound of Example 1 but replacing Compound 1b with Compound 15b. Purification of the crude obtained from the usual work-up was done by automated flash chromatography (Horizon®-Biotage) eluting with PE-EtOAc 7:3) afforded the title compound as yellow solid. Yield: 33%.

¹H-NMR (CDCl₃, δ): 2.49 (s, 3H), 2.59-2.62 (m, 2H), 2.95-2.99 (m, 2H), 3.57-3.62 (m, 4H), 6.27 (s, 1H), 6.50 (s, 1H), 6.61-6.64 (m, 1H), 7.44-7.51 (m, 3H), 7.80-7.82 (m, 2H), 8.10-8.12 (m, 1H).

MS: [M+H]⁺=377.41

### Example 16

### 1-(3-nitro-2-pyridyl)-4-[(5-methyl-1-benzofuran-2-yl)-methylene]-piperidine

### 1-(t-butoxycarbonyl)-4-[(5-methyl-1-benzofuran-2-yl)-methylene]-piperidine (Compound 16a)

A mixture of Compound 14a (200 mg, 0.681 mmol), 2-bromo-4-methylphenol (0.099 ml, 0.818 mmol), tetrakis(triphenylphosphine)palladium(0) (39.3 mg, 0.034 mmol), tetrabutylammonium fluoride (178 mg, 0.681 mmol), sodium acetate (112 mg, 1.36 mmol) in 5 ml of DMF degassed flowing nitrogen, was heated in a microwave oven (Discovery®-CEM) at 120° C. for 10 min. Quench with water and extraction with EtOAc afforded a residue, which was purified by automated flash chromatography (Horizon®-Biotage) eluting with a PE-EtOAC gradient from 1:0 to 7:3 to afford 60 mg of the title product.

MS: [M+H]⁺=328.23

### 4-[(5-methyl-1-benzofuran-2-yl)-methylene]-piperidine (Compound 16b)

A solution of Compound 16a (100 mg, 0.288 mmol), and trifluoroacetic acid (0.222 ml, 10 ml) in 5 ml of CH₂Cl₂ was stirred overnight at r.t. After the usual work-up, a colourless oil was obtained, used without further purification in the next step.

### 1-(3-nitro-2-pyridyl)-4-[(5-methyl-1-benzofuran-2-yl)-methylene]-piperidine

The title compound was synthesised according to the procedure described for the compound of Example 7 but using Compound 16b in place of Compound 7a. After the usual work-up procedure, the crude was purified by automated flash chromatography (Horizon®-Biotage) eluting with PE-EtOAc gradient from 9:1 to 7:3, affording the title compound. Yield: 3.91%

¹H-NMR (CDCl₃, δ): 2.4 (s, 3H), 2.6 (m, 2H), 3.0-3.1 (m, 2H), 3.5-3.65 (m, 4H), 6.35 (s, 1H), 6.5 (s, 1H), 6.85 (m, 1H), 7.10 (m, 1H), 7.2 (m, 1H), 7.3-7.4 (m, 2H), 8.2 (m, 1H), 8.4 (m, 1H).

MS: [M+H]⁺=350.38

### Example 17

### 1-(3-nitro-2-pyridyl)-4-[(1-benzofuran-2-yl)-methylene]-piperidine

The title compound was prepared following the procedure reported for the compound of Example 9, but using 2-iodophenol instead of 2-iodo-4-phenylphenol. After the usual work-up procedure, the crude was purified by automated flash chromatography (Horizon®-Biotage) eluting with PE-EtOAc 8:2, affording the title compound (33%).

¹H-NMR (CDCl₃): 2.6 (m, 2H), 3.0-3.1 (m, 2H), 3.5-3.65 (m, 4H), 6.35 (s, 1H), 6.5 (s, 1H), 6.75 (m, 1H), 7.2-7.3 (m, 2H), 7.4-7.5 (m, 1H), 7.5-7.6 (m, 1H), 8.2 (m, 1H), 8.4 (m, 1H).

MS: [M+H]⁺=336.36

### Example 18

### 1-(6-methyl-3-nitro-2-pyridyl)-4-[(5-phenyl-1,2,4-oxadiazol-3-yl)-methylene]-piperidine

### Diethyl (5-phenyl-1,2,4-oxadiazol-3-yl)-methylphosphonate (Compound 18a)

3-Chloromethyl-5-phenyl-1,2,4-oxadiazole (150 mg, 0.771 mmol) was heated in triethyl phosphite at 120° C. for 8 h. Then KI (30 mg) was added stirring at 160° C. for 8 h. The crude was purified by automated flash chromatography (Horizon®-Biotage) eluting with CH₂Cl₂-MeOH 95:5 affording 228 mg of the title product as yellowish oil.

MS: [M+H]⁺=297.31

### 1-(6-methyl-3-nitro-2-pyridyl)-4-[(5-phenyl-1,2,4-oxadiazol-3-yl)-methylene]-piperidine

The title product was synthesized following the same procedure reported above for the compound of Example 1 but using Compound 18a instead of Compound 1b. Purification was carried out by automated flash chromatography (Horizon®-Biotage) eluting with PE-EtOAc 7:3 affording a yellow solid. Yield: 14%.

¹H-NMR (CDCl₃, δ): 2.51 (s, 3H), 2.63-2.65 (m, 2H), 3.23-3.25 (m, 2H), 3.59-3.64 (m, 4H), 6.36 (s, 1H), 6.62-6.64 (m, 1H), 7.54-7.64 (m, 3H), 8.11-8.13 (m, 1H), 8.17-8.19 (m, 2H).

MS: [M+H]⁺=378.43

### Example 19

### 1-(6-methyl-3-nitro-2-pyridyl)-4-[(3-phenyl-isoxazol-5-yl)-methylene]-piperidine

### Diethyl (3-phenylisoxazol-5-yl)-methylphosphonate (Compound 19a)

The title compound (87.4%) was synthesized following exactly the same procedure described for Compound 18a but substituting 5-(chloromethyl)-3-phenylisoxazole for 3-chloromethyl-5-phenyl-1,2,4-oxadiazole.

MS: [M+H]⁺=296.38

### 1-(6-methyl-3-nitro-2-pyridyl)-4-[(3-phenyl-isoxazol-5-yl)-methylene]-piperidine

The title product was synthesized following the same procedure reported above for the Compound of Example 1 but using Compound 19a instead of Compound 1b. Purification was carried out by automated flash chromatography (Horizon®-Biotage) eluting with PE-EtOAc 7:3 affording a yellow solid. Yield: 10%.

¹H-NMR (CDCl₃, δ): 2.52 (s, 3H), 2.61-2.63 (m, 2H), 2.98-3.01 (m, 2H), 3.57-3.65 (m, 4H), 6.32 (s, 1H), 6.44 (s, 1H), 6.63-6.65 (m, 1H), 7.48-7.51 (m, 3H), 7.83-7.85 (m, 2H), 8.11-8.13 (m, 1H).

MS: [M+H]⁺=378.43

### Preparation of Intermediates for Use in Examples 20-22, 25-29, 31, 32 and 36

Following the methodologies described above for Compound 1b (Method A) and Compound 18a (Method B) the following phosphonates were synthesized. Purification was performed by automated flash chromatography (Horizon®-Biotage) eluting with CH₂Cl₂-MeOH 95:5 or 98:2:

| | | | Method/ |
|---|---|---|---|
| Comp. | Structure | Starting material | Yield |
| 20 | 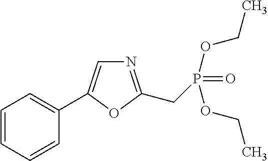 | Diethyl (5-phenyl-1,3-oxazol-2- yl)-methylphosphonate | A/75% |
| 21 | 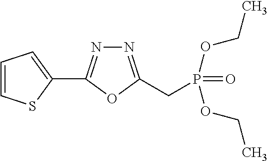 | Diethyl [5-(2-thienyl)-1,3,4- oxadiazol-2-yl]- methylphosphonate | A/51% |
| 22 | 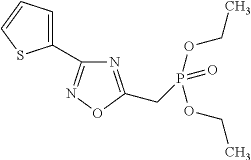 | Diethyl [3-(2-thienyl)-1,2,4- oxadiazol-5-yl]- methylphosphonate | A/88% |
| 23 | 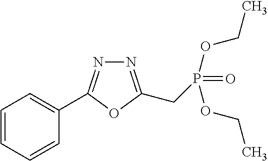 | Diethyl (5-phenyl-1,3,4- oxadiazol-2-yl)- methylphosphonate | A/94% |
| 24 | 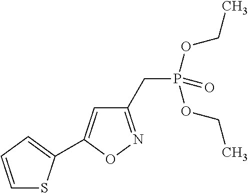 | Diethyl [5-(2-thienyl)-isoxazol-3- yl]-methylphosphonate | A/100% |
| 25 | 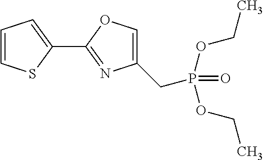 | Diethyl [2-(2-thienyl)-1,3-oxazol- 4-yl]-methylphosphonate | A/79.5% |
| 26 | 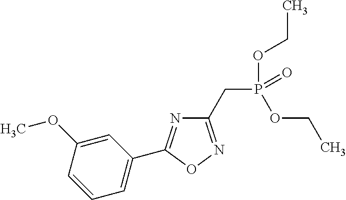 | Diethyl [5-(3-methoxyphenyl)- 1,2,4-oxadiazol-3-yl]- methylphosphonate | A/97% |
| 27 | 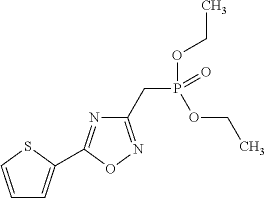 | Diethyl [5-(2-thienyl)-1,2,4- oxadiazol-3-yl]- methylphosphonate | A/91% |
| 28 | 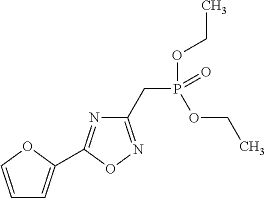 | Diethyl [5-(2-furyl)-1,2,4- oxadiazol-3-yl]- methylphosphonate | A/98% |
| 29 | 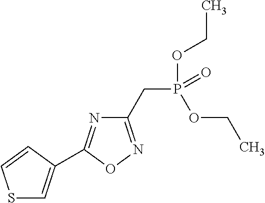 | Diethyl [5-(3-thienyl)-1,2,4- oxadiazol-3-yl]- methylphosphonate | A/97% |
| 30 | 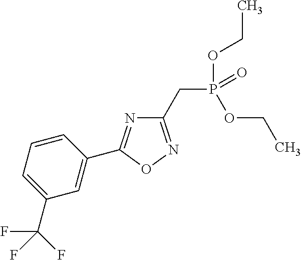 | Diethyl [5-(3- trifluoromethylphenyl)-1,2,4- oxadiazol-3-yl]- methylphosphonate | A/99.5% |

### Example 20

### 1-(6-methyl-3-nitro-2-pyridyl)-4-[(5-phenyl-1,3-oxazol-2-yl)-methylene]-piperidine

The title product was synthesised following the same procedure reported above for the compound of Example 1 but using Compound 20 instead of Compound 1b. Purification was carried out by automated flash chromatography (Horizon®-Biotage) eluting with PE-EtOAc 8:2. Further purification was got by preparative RP LC-MS chromatography, using MS-C18 XTerra column 30×50 mm eluting with ammonium bicarbonate 20 mM pH 8 buffer—acetonitrile gradient. Yield: 8.3%.

¹H-NMR (CDCl₃, δ): 2.52 (s, 3H), 2.66-2.69 (m, 2H), 3.23-3.25 (m, 2H), 3.62-3.66 (m, 4H), 6.46 (s, 1H), 6.64-6.66 (m, 1H), 7.38-7.50 (m, 3H), 7.66-7.67 (m, 2H), 8.12-8.14 (m, 1H).

MS: [M+H]⁺=377.52

### Example 21

### 1-(6-methyl-3-nitro-2-pyridyl)-4-{[5-(2-thienyl)-1,3,4-oxadiazol-2-yl]-methylene}-piperidine

### 1-(t-butoxycarbonyl)-4-{[5-(2-thienyl)-1,3,4-oxadiazol-2-yl]-methylene}-piperidine (Compound 21a)

The title product was synthesised following the same procedure reported above for the compound of Example 1 but using Compound 21 instead of Compound 1b and 1-tert-butoxycarbonyl-4-piperidone instead of 1-(6-methyl-3-nitropyridin-2-yl)piperidin-4-one. Purification was carried out by automated flash chromatography (Horizon®-Biotage) eluting with PE-EtOAc 7:3 giving the title product (82%).

MS: [M+H]⁺=348.44

### 4-{[5-(2-thienyl)-1,3,4-oxadiazol-2-yl]-methylene}-piperidine (Compound 21b)

A solution of Compound 21a (100 mg, 0.288 mmol), and trifluoroacetic acid (0.222 ml, 10 ml) was stirred overnight at r.t. After the usual work-up, was obtained a colourless oil, used without further purification in the next step.

MS: [M+H]⁺=248.50

### 1-(6-methyl-3-nitro-2-pyridyl)-4-{[5-(2-thienyl)-1,3,4-oxadiazol-2-yl]-methylene}-piperidine

The title product was synthesised following the same procedure reported above for the compound of Example 7, but using Compound 21b instead of Compound 7a. Purification was carried out by automated flash chromatography (Horizon®-Biotage) eluting with PE-EtOAc 9:1 giving the title product as a yellow solid. Yield: 82%.

¹H-NMR (CDCl₃, δ): 2.56 (s, 3H), 2.67-2.70 (m, 2H), 3.20-3.23 (m, 2H), 3.63-3.66 (m, 4H), 6.34 (s, 1H), 6.66-6.69 (m, 1H), 7.19-7.21 (m, 1H), 7.56-7.58 (m, 1H), 7.77-7.78 (m, 1H), 8.14-8.16 (m, 1H).

MS: [M+H]⁺=384.52

### Example 22

### 1-(6-methyl-3-nitro-2-pyridyl)-4-{[3-(2-thienyl)-1,2,4-oxadiazol-5-yl]-methylene}-piperidine

### 1-(t-butoxycarbonyl)-4-{[3-(2-thienyl)-1,2,4-oxadiazol-5-yl]-methylene}-piperidine (Compound 22a)

The title compound was prepared as described for Compound 21a, but using Compound 22 instead of Compound 21. Purification by automated flash chromatography (Horizon®-Biotage) eluting with PE-EtOAc 7:3 gave the title product. Yield: 50.2%.

¹H-NMR (CDCl₃, δ): 1.52 (s, 9H), 2.46-2.49 (m, 2H), 3.09-3.12 (m, 2H), 3.58-3.62 (m, 4H), 6.35 (s, 1H), 7.17-7.19 (m, 1H), 7.51-7.53 (m, 1H), 7.82-7.83 (m, 1H).

MS: [M+H]⁺=348.45

### 4-{[3-(2-thienyl)-1,2,4-oxadiazol-5-yl]-methylene}-piperidine (Compound 22b)

Prepared following the procedure reported above for Compound 21b, but using Compound 22a instead Compound 21a. Obtained as colourless oil and used in the next step without further purification.

MS: [M+H]⁺=248.35

### 1-(6-methyl-3-nitro-2-pyridyl)-4-{[3-(2-thienyl)-1,2,4-oxadiazol-5-yl]-methylene}-piperidine

The title product was synthesised following the same procedure reported above for the compound of Example 7 but using Compound 22b instead of Compound 7a. Purification was carried out by automated flash chromatography (Horizon®-Biotage) eluting with PE-EtOAc 8:2 followed by preparative RP LC-MS chromatography, using MS-C18 XTerra column 30×50 mm eluting with ammonium bicarbonate 20 mM pH 8 buffer-acetonitrile gradient and afforded the title product as a yellow solid. Yield: 22%.

¹H-NMR (CDCl₃, δ): 2.52 (s, 3H), 2.66-2.69 (m, 2H), 3.28-3.31 (m, 2H), 3.61-3.65 (m, 4H), 6.40 (s, 1H), 6.65-6.67 (m, 1H), 7.17-7.19 (m, 1H), 7.52-7.53 (m, 1H), 7.83-7.84 (m, 1H), 8.12-8.14 (m, 1H).

MS: [M+H]⁺=384.53

### Example 23

### 1-(6-methyl-3-nitro-2-pyridyl)-4-[(1-phenyl-1H-1,2,3-triazol-5-yl)-methylene]-piperidine

### 1-(t-butoxycarbonyl)-4-[(1-phenyl-1H-1,2,3-triazol-5-yl)-methylene]-piperidine (Compound 23a)

To a solution of Compound 14b (70 mg, 0.32 mmol) and phenyl azide (49 mg, 0.41 mmol) in anhydrous DMF (3 ml) placed in a sealable vessel was added [{Cp*Ru(μ₃-Cl)}₄] (15 mg, 0.055 mmol; prepared as described in J. Am. Chem. Soc., 1989, 111, 1698-1719). After having flushed with nitrogen, the recipient was sealed and put in a microwave oven (CEM) and stirred at 110° C. for 45 min. After cooling, the reaction mixture was diluted with EtOAc and washed with water, dried (Na₂SO₄) and evaporated to dryness in vacuo. The crude was purified by automated flash chromatography (Horizon®-Biotage) eluting with PE-EtOAc gradient from 8:2 to 5:5 affording the title compound. Yield: 93%.

¹H-NMR (CDCl₃, δ): 1.5 (s, 9H), 2.34-2.36 (m, 2H), 2.48-2.50 (m, 2H), 3.43-3.52 (m, 4H), 6.00 (s, 1H), 7.53-7.57 (m, 5H), 7.74 (s, 1H)

MS: [M+H]⁺=341.25

### 4-[(1-phenyl-1H-1,2,3-triazol-5-yl)-methylene]-piperidine (Compound 23b)

Prepared following the procedure reported above for Compound 22b but using Compound 23a instead of Compound 22a. Obtained as colourless oil and used in the next step without further purification.

MS: [M+H]⁺=240.18

### 1-(6-methyl-3-nitro-2-pyridyl)-4-[(1-phenyl-1H-1,2,3-triazol-5-yl)-methylene]-piperidine

The title product was synthesized following the same procedure reported above for the compound of Example 7, but using Compound 23b instead of Compound 7a. Purification was carried out by automated flash chromatography (Horizon®SP1-Biotage) eluting with PE-EtOAc gradient from 8:2 to 5:5, giving the title product. Yield: 50%.

¹H-NMR (CDCl₃δ): 2.5 (s, 3H), 2.50-2.55 (m, 2H), 2.68-2.71 (m, 2H), 3.49-3.57 (m, 4H), 6.05 (s, 1H), 6.64 (d, 1H), 7.28-7.59 (m, 5H), 7.76 (s, 1H), 8.11 (dd, 1H)

MS: [M+H]⁺=377.28

### Example 24

### 1-(6-methyl-3-nitro-2-pyridyl)-4-[(1-benzofuran-2-yl)-methylene]-piperidine

### 1-(6-methyl-3-nitro-2-pyridyl)-4-(3-trimethysilyl-prop-2-ynylidene)-piperidine (Compound 24a)

The title compound was synthesized following the procedure described for Compound 3b, but replacing 1-(3-nitro-2-pyridyl)-4-oxo-piperidine with 1-(6-methyl-3-nitro-2-pyridyl)-4-oxo-piperidine. The crude residue obtained from a standard work-up procedure was used in the next step without further purification.

MS: [M+H]⁺=330.27

### 1-(6-methyl-3-nitro-2-pyridyl)-4-(prop-2-ynylidene)-piperidine (Compound 24b)

The title compound was synthesized following the procedure described for Compound 3c, but using Compound 24a instead of Compound 1b. Purification by automated flash liquid chromatography (Horizon®-Biotage) eluting with a PE-Acetone gradient from 97:3 to 9:1 afforded the title compound. Yield: 29%.

MS: [M+H]⁺=258.08

### 1-(6-methyl-3-nitro-2-pyridyl)-4-[(1-benzofuran-2-yl)-methylene]-piperidine

To a solution of Compound 24b (50 mg, 0.19 mmol) and 2-iodophenol (51.2 mg, 0.23 mmol) in anhydrous DMF (5 ml) placed in a sealable vessel was added tetrakis(triphenylphosphine)palladium(0) (11.2 mg, 0.01 mmol), tetrabutylammonium fluoride (50.7 mg, 0.19 mmol) and sodium acetate (31.8 mg, 0.38 mmol). After having flushed with nitrogen, the recipient was sealed, put in a microwave oven (Biotage) and stirred at 120° C. for 10 min. After cooling to r.t., the reaction mixture was diluted with EtOAc and washed with water, dried (Na₂SO₄) and evaporated to dryness in vacuo. The crude was purified by automated flash chromatography (SP1®-Biotage) eluting with PE-EtOAc gradient from 95:5 to 7:3 affording the title compound. Yield: 44.3%.

¹H-NMR (CDCl₃δ): 2.5 (s, 3H), 2.55-2.65 (m, 2H), 3.05-3.1 (m, 2H), 3.55-3.65 (m, 4H), 6.3 (s, 1H), 6.55 (s, 1H), 6.60-6.65 (d, 1H), 7.2-7.3 (m, 2H), 7.45-7.50 (d, 1H), 7.50-7.55 (d, 1H), 8.1 (d, 1H).

MS: [M+H]⁺=350.38

### Example 25

### 1-(6-methyl-3-nitro-2-pyridyl)-4-[5-phenyl-1,3,4-oxadiazol-2-yl)-methylene]-piperidine

### 1-(t-butoxycarbonyl)-4-[5-phenyl-1,3,4-oxadiazol-2-yl)-methylene]-piperidine (Compound 25a)

The title compound was synthesised according to the procedure described for the compound of Example 1, but using Compound 23 instead of Compound 1b and reacting it with 1-tert-butoxycarbonyl-4-oxo-piperidine. After the usual work-up procedure, the crude was purified by automated flash chromatography (Horizon®-Biotage) eluting with PE-EtOAc 7:3 affording the title compound. Yield: 53.5%.

¹H-NMR (CDCl₃, δ): 1.52 (s, 9H), 2.46-2.49 (m, 2H), 3.03-3.06 (m, 2H), 3.58-3.60 (m, 4H), 6.31 (s, 1H), 7.53-7.56 (m, 3H), 8.07-8.09 (m, 2H).

MS: [M+H]⁺=342.33

### 4-[5-phenyl-1,3,4-oxadiazol-2-yl)-methylene]-piperidine (Compound 25b)

Prepared following the procedure reported above for Compound 21b but using Compound 25a instead of Compound 21a. Obtained as colourless oil and used in the next step without further purification.

MS: [M+H]⁺=242.42

### 1-(6-methyl-3-nitro-2-pyridyl)-4-[5-phenyl-1,3,4-oxadiazol-2-yl)-methylene]-piperidine

The title product was synthesised following the same procedure reported above for the compound of Example 7, but using Compound 25b instead of Compound 7a. Purification was carried out by automated flash chromatography (Horizon®-Biotage) eluting with PE-EtOAc 8:2 giving the title product. Yield: 41%.

¹H-NMR (CDCl₃, δ): 2.51 (s, 3H), 2.66-2.68 (m, 2H), 3.21-3.34 (m, 2H), 3.60-3.66 (m, 4H), 6.37 (s, 1H), 6.64-6.66 (m, 1H), 7.54-7.55 (m, 3H), 8.08-8.14 (m, 3H).

MS: [M+H]⁺=378.51

### Example 26

### 1-(6-methyl-3-nitro-2-pyridyl)-4-{[5-(2-thienyl)-isoxazol-3-yl]-methylene}-piperidine

### 1-(t-butoxycarbonyl)-4-{[5-(2-thienyl)-isoxazol-3-yl]-methylene}-piperidine (Compound 26a)

The title product was synthesised following the same procedure reported above for the compound of Example 1, but using Compound 24 instead of Compound 1b and 1-tert-butoxycarbonyl-4-oxo-piperidine instead of 1-(6-methyl-3-nitropyridin-2-yl)-4-oxo-piperidine. Purification was carried out by automated flash chromatography (Horizon®-Biotage) eluting with PE-EtOAc 8:2 giving the title product as a white solid. Yield: 67%.

MS: [M+H]⁺=347.56

### 4-{[5-(2-thienyl)-isoxazol-3-yl]-methylene}-piperidine (Compound 26b)

Prepared following the procedure reported above for Compound 21b, but using Compound 26a instead Compound 21a. Obtained as colourless oil and used in the next step without further purification. Yield: 70%.

MS: [M+H]⁺=247.36

### 1-(6-methyl-3-nitro-2-pyridyl)-4-{[5-(2-thienyl)-isoxazol-3-yl]-methylene}-piperidine

The title product was synthesised following the same procedure reported above for the compound of Example 7 but using Compound 26b instead of Compound 7a. Purification was carried out by automated flash chromatography (Horizon®SP1-Biotage) eluting with PE-EtOAc 8:2 giving the title product as a yellow solid. Yield: 95%.

¹H-NMR (CDCl₃, δ): 2.51 (s, 3H), 2.59-2.62 (m, 2H), 2.92-2.95 (m, 2H), 3.58-3.59 (m, 4H), 6.23 (s, 1H), 6.36 (s, 1H), 6.61-6.63 (m, 1H), 7.14-7.16 (m, 1H), 7.46-7.48 (m, 1H), 7.53-7.54 (m, 1H), 8.10-8.12 (m, 1H).

MS: [M+H]⁺=383.46

### Example 27

### 1-(6-methyl-3-nitro-2-pyridyl)-4-{[2-(2-thienyl)-1,3-oxazol-4-yl]-methylene}-piperidine

### 1-(t-butoxycarbonyl)-4-{[2-(2-thienyl)-1,3-oxazol-4-yl]-methylene}-piperidine (Compound 27a)

The title product was synthesized following the same procedure reported above for the compound of Example 1, but using Compound 25 instead of Compound 1b and 1-tert-butoxycarbonyl-4-piperidone instead of 1-(6-methyl-3-nitropyridin-2-yl)piperidin-4-one. Purification was carried out by automated flash chromatography (Horizon®-Biotage) eluting with PE-EtOAc 8:2 giving the title product as white solid. Yield: 23.5%.

MS: [M+H]⁺=347.51

### 4-{[2-(2-thienyl)-1,3-oxazol-4-yl]-methylene}-piperidine (Compound 27b)

Prepared following the procedure reported above for Compound 21b but using Compound 27a instead Compound 21a. Obtained as colourless oil and used in the next step without further purification. Yield: 94%.

MS: [M+H]⁺=247.45

### 1-(6-methyl-3-nitro-2-pyridyl)-4-{[2-(2-thienyl)-1,3-oxazol-4-yl]-methylene}-piperidine

The title product was synthesised following the same procedure reported above for the compound of Example 7, but using Compound 27b instead of Compound 7a. Purification was carried out by automated flash chromatography (Horizon®-Biotage) eluting with PE-EtOAc 8:2 giving the title product as yellow solid. Yield: 22%.

¹H-NMR (CDCl₃, δ): 2.50 (s, 3H), 2.60-2.63 (m, 2H), 2.96-2.99 (m, 2H), 3.56-3.61 (m, 4H), 6.19 (s, 1H), 6.59-6.61 (m, 1H), 7.13-7.16 (m, 1H), 7.45-7.48 (m, 1H), 7.54 (s, 1H), 7.72-7.73 (m, 1H), 8.09-8.11 (m, 1H).

MS: [M+H]⁺=383.48

### Example 28

### 1-(6-methyl-3-nitro-2-pyridyl)-4-{[5-(3-methoxyphenyl)-1,2,4-oxadiazol-3-yl]-methylene}-piperidine

The title compound was synthesized following the same procedure reported above for the compound of Example 1 but using Compound 26 instead of 1b. Purification was carried out by automated flash chromatography (Horizon®-Biotage) eluting with PE-EtOAc 8:2 giving the title product as yellow solid. Yield: 55%.

¹H-NMR (CDCl₃, δ): 2.50 (s, 3H), 2.62-2.65 (m, 2H), 3.21-3.24 (m, 2H), 3.58-3.64 (m, 4H), 3.93 (s, 3H) 6.35 (s, 1H), 6.61-6.63 (m, 1H), 7.14-7.17 (m, 1H), 7.44-7.48 (m, 1H), 7.67 (s, 1H), 7.76-7.78 (m, 1H), 8.10-8.12 (m, 1H).

MS: [M+H]⁺=408.54

### Example 29

### 1-(6-methyl-3-nitro-2-pyridyl)-4-{[5-(2-thienyl)-1,2,4-oxadiazol-3-yl]-methylene}-piperidine

The title compound was synthesized following the same procedure reported above for the compound of Example 1, but using Compound 27 instead of Compound 1b. Purification was carried out by automated flash chromatography (Horizon®-Biotage) eluting with PE-EtOAc 8:2 giving the title product as yellow solid. Yield: 57%.

¹H-NMR (CDCl₃, δ): 2.50 (s, 3H), 2.61-2.64 (m, 2H), 3.18-3.20 (m, 2H), 3.58-3.62 (m, 4H), 6.31 (s, 1H), 6.61-6.63 (m, 1H), 7.21-7.24 (m, 1H), 7.65-7.67 (m, 1H), 7.92-7.93 (m, 1H), 8.10-8.12 (m, 1H).

MS: [M+H]⁺=384.61

### Example 30

### 1-(6-methyl-3-nitro-2-pyridyl)-4-[(4-hydroxy-1-benzofuran-2-yl)-methylene]-piperidine

The title compound was prepared as reported for the compound of Example 17, but replacing 2-iodophenol with 2,3-dihydroxyiodobenzene. Purification was carried out by automated flash chromatography (SP1®-Biotage) eluting with a PE-EtOAc gradient from 95:5 to 7:3 giving the title product as orange oil. Yield: 46%.

¹H-NMR (CDCl₃, δ): 2.5 (s, 3H), 2.55-2.65 (m, 2H), 3.05-3.1 (m, 2H), 3.55-3.65 (m, 4H), 5.0 (s, 1H), 6.3 (s, 1H), 6.55-6.65 (m, 3H), 7.0-7.15 (m, 2H), 8.1 (d, 1H).

MS: [M+H]⁺=366.38

### Example 31

### 1-(6-methyl-3-nitro-2-pyridyl)-4-{[5-(2-furyl)-1,2,4-oxadiazol-3-yl]-methylene}-piperidine

The title compound was synthesized following the same procedure reported above for the compound of Example 1 but using Compound 28 instead of Compound 1b. Purification was carried out by automated flash chromatography (Horizon®-Biotage) eluting with PE-EtOAc 8:2 giving the title product as a yellow solid. Yield: 55%.

¹H-NMR (CDCl₃, δ): 2.49 (s, 3H), 2.61-2.65 (m, 2H), 3.18-3.21 (m, 2H), 3.58-3.62 (m, 4H), 6.32 (s, 1H), 6.61-6.66 (m, 2H), 7.33-7.34 (m, 1H), 7.72 (s, 1H), 8.10-8.12 (m, 1H).

MS: [M+H]⁺=368.41

### Example 32

### 1-(6-methyl-3-nitro-2-pyridyl)-4-{[5-(3-thienyl)-1,2,4-oxadiazol-3-yl]-methylene}-piperidine

The title compound was synthesized following the same procedure reported above for the compound of Example 1, but using Compound 29 instead of Compound 1b. Purification was carried out by automated flash chromatography (Horizon®-Biotage) eluting with PE-EtOAc 8:2 giving the title product as yellow solid. Yield: 53%.

¹H-NMR (CDCl₃, δ): 2.50 (s, 3H), 2.61-2.64 (m, 2H), 3.19-3.22 (m, 2H), 3.58-3.63 (m, 4H), 6.33 (s, 1H), 6.61-6.63 (m, 1H), 7.48-7.50 (m, 1H), 7.71-7.72 (m, 1H), 8.10-8.12 (m, 1H), 8.23-8.24 (m, 1H).

MS: [M+H]⁺=384.53

### Example 33

### 1-(6-methyl-3-nitro-2-pyridyl)-4-[(1H-benzimidazol-2-yl)-methylene]-piperidine

### (1H-Benzimidazol-2-ylmethyl)(triphenyl)phosphonium chloride (Compound 33a)

A mixture of 2-chloromethyl-benzimidazole (500 mg, 3 mmol) and triphenylphosphine (1.61 g, 2 mmol) in 25 ml of toluene was refluxed for 16 h. The creamy solid, which precipitated, was filtered off, washing on the funnel with toluene. 762 mg (59%) of the title product was obtained.

MS: [M]⁺=429.15

### 1-(6-methyl-3-nitro-2-pyridyl)-4-[(1H-benzimidazol-2-yl)-methylene]-piperidine

The title compound was synthesized following the same procedure reported above for the compound of Example 1 but using Compound 33a instead of Compound 1b. Purification was carried out by automated flash chromatography (Horizon®-Biotage) eluting with a PE-EtOAc gradient from 95:5 to 6:4 giving the title product as a yellow solid. Yield: 6%.

¹H-NMR (CDCl₃, δ): 2.45-2.55 (m, 5H), 3.15-3.25 (m, 2H), 3.45-3.65 (m, 4H), 6.33 (s, 1H), 6.63 (d, J=8 Hz, 1H) 7.30-7.37 (m, 2H), 7.67-7.75 (m, 2H), 8.07 (d, J=8 Hz, 1H).

MS: [M+H]⁺=350.2

### Example 34

### 1-(6-methyl-3-nitro-2-pyridyl)-4-[(1-benzothien-2-yl)-methylene]-piperidine

### 1-(t-butoxycarbonyl)-4-bromomethylene-piperidine (Compound 34a)

LiHMDS (1 M in THF, 7.38 ml, 7.38 mmol) was dropped into a suspension of bromomethyltriphenylphosphonium bromide (3.22 g, 7.38 mmol) at −15° C. under nitrogen atmosphere. After 15 min. under stirring at the same temperature, 1-(t-butoxycarbonyl)-4-oxo-piperidine (1.4 g, 7.03 mmol) dissolved in THF (10 ml) was added. Stirring was maintained and after 2 h at r.t., the reaction mixture was quenched with water and with EtOAc. The combined extracts were washed, dried over Na₂SO₄ and evaporated to dryness. The crude residue was purified by flash chromatography (EtOAc-PE 98:2) affording the title product (1.27 g).

MS: [M+H]⁺=276.2

### 1-(t-butoxycarbonyl)-4-[(1-benzothien-2-yl)-methylene]-piperidine (Compound 34b)

A mixture of Compound 34a (250 mg, 0.905 mmol), benzo[b]thiophene-2-boronic acid (483 mg, 2.71 mmol), tetrakis(triphenylphosphine)palladium(0) (52 mg, 0.045 mmol), K₂CO₃ (375 mg, 2.71 mmol), LiCl (230 mg, 5.43 mmol) in 85 ml of dimethoxyethane was refluxed for 4 h. The reaction mixture was quenched with water and diluted with EtOAc. The combined extracts were washed, dried over Na₂SO₄ and evaporated to dryness. The crude residue was purified by flash chromatography (EtOAc-Petroleum Ether 95:5) affording the title product (271 mg).

MS: [M+H]⁺=330.5

### 4-[(1-benzothien-2-yl)-methylene]-piperidine (Compound 34c)

A solution of Compound 34b (271 mg, 0.82 mmol), CF₃COOH (0.634 ml, 8.23 mmol) in 5 ml CHCl₃ was stirred for 24 h, then the solvent was evaporated affording the crude title product (205 mg), which was used without further purification in the next step.

MS: [M+H]⁺=230.5

### 1-(6-methyl-3-nitro-2-pyridyl)-4-[(1-benzothien-2-yl)-methylene]-piperidine

A solution of 2-chloro-3-nitro-6-picoline (152 mg, 0.88 mmol), Compound 34c (202 mg, 0.88 mmol) and triethylamine (0.368 ml, 2.64 mmol) in 5 ml of dimethylacetamide was stirred for 24 h at r.t. Water was dropped into the solution until precipitation of a solid; the solid was filtered and dried in a oven at 50° C. at 1 mbar of residual pressure, obtaining 231 mg of the title compound as yellow powder.

¹H-NMR (CDCl₃, δ): 2.50 (s, 3H), 2.55-2.65 (m, 2H), 2.87-2.97 (m, 2H), 3.5-3.68 (m, 4H), 6.56 (s, 1H), 6.61 (d, J=8 Hz, 1H), 7.15 (s, 1H), 7.25-7.40 (m, 2H), 7.70-7.85 (m, 2H), 8.10 (d, J=8 Hz, 1H).

MS: [M+H]⁺=366.5

### Example 35

### 1-(6-methyl-3-nitro-2-pyridyl)-4-[(6-hydroxymethyl-1-benzofuran-2-yl)-methylene]-piperidine

The title compound was prepared as reported for the compound of Example 17 but replacing 2-iodophenol with 3-hydroxy-4-iodobenzyl alcohol. Purification was carried out by automated flash chromatography (SP1®-Biotage) eluting with a PE-EtOAc gradient from 95:5 to 7:3 giving the title product as orange oil. Yield: 46%.

¹H-NMR (CDCl₃, δ): 2.5 (s, 3H), 2.55-2.65 (m, 2H), 3.0-3.1 (m, 2H), 3.55-3.65 (m, 4H), 4.8 (s, 2H), 6.30 (s, 1H), 6.5 (s, 1H), 6.55-6.65 (d, 1H), 7.2-7.3 (d, 1H), 7.50-7.55 (m, 2H), 8.1 (d, 1H).

MS: [M+H]⁺=380.41

### Example 36

### 1-(6-methyl-3-nitro-2-pyridyl)-4-{[5-(3-trifluoromethylphenyl)-1,2,4-oxadiazol-3-yl]-methylene}-piperidine

The title compound was synthesized following the same procedure reported above for the compound of Example 1, but using Compound 30 instead of Compound 1b. Purification was carried out by automated flash chromatography (Horizon®-Biotage) eluting with PE-EtOAc 8:2 giving the title product as a yellow solid. Yield: 51%.

¹H-NMR (CDCl₃, δ): 2.50 (s, 3H), 2.63-2.66 (m, 2H), 3.22-3.24 (m, 2H), 3.60-3.64 (m, 4H), 6.36 (s, 1H), 6.62-6.64 (m, 1H), 7.70-7.74 (m, 1H), 7.87-7.89 (m, 1H), 8.11-8.13 (m, 1H), 8.35-8.37 (m, 1H), 8.44 (s, 1H).

MS: [M+H]⁺=446.45

### Example 37

### 1-(6-methyl-3-nitro-2-pyridyl)-4-[(3-methyl-1,2,4-oxadiazol-5-yl)-methylene]-piperidine

### Diethyl (3-methyl-1,2,4-oxadiazol-5-yl)-methylphosphonate (Compound 37a)

Into a solution of LiHMDS (1 M in THF, 0.622 ml, 0.622 mmol) in anhydrous THF (2.5 ml) stirred at −10° C. under nitrogen atmosphere was dropped a solution of diethyl phosphite (0.080 ml, 0.622 mmol) in 2.5 ml of THF and the reaction mixture was kept at −10° C. per 20 min. Afterwards, 5-chloromethyl-3-methyl-1,2,4-oxadiazole (100 mg, 0.754 mmol) dissolved in THF (2.5 ml) was dropped in and stirring continued for 2 hours at −10° C. and overnight at r.t. The reaction mixture was used directly in the next step. Yield: 100%.

MS: [M+H]⁺=235.42

### 1-(t-butoxycarbonyl)-4-[(3-methyl-1,2,4-oxadiazol-5-yl)-methylene]-piperidine (Compound 37b)

The title compound was prepared following the procedure described above for the compound of Example 1, but using Compound 37a instead of Compound 1b and using 1-tert-butoxycarbonyl-4-piperidone instead of 1-(6-methyl-3-nitropyridin-2-yl)piperidin-4-one. The crude was used in the following step without any further purification. Yield: 100%.

MS: [M+H]⁺=280.4

### 4-[(3-methyl-1,2,4-oxadiazol-5-yl)-methylene]-piperidine (Compound 37c)

The title compound was prepared following the procedure described above for Compound 14d, but using Compound 37b instead of Compound 14c. The crude was used in the following step without any further purification. Yield: 100%.

MS: [M+H]⁺=180.3

### 1-(6-methyl-3-nitro-2-pyridyl)-4-[(3-methyl-1,2,4-oxadiazol-5-yl)-methylene]-piperidine

The title compound was prepared following the procedure described above for the compound of Example 34, but using Compound 37c instead of Compound 34c. Purification was carried out by automated flash chromatography (Horizon®-Biotage) eluting with a PE-EtOAc gradient from 10:0 to 8:2, giving the title product as yellow solid. Yield: 17%.

¹H-NMR (CDCl₃, δ): 2.04 (s, 3H), 2.16 (s, 3H), 2.25-2.35 (m, 2H), 2.80-2.90 (m, 2H), 3.20-3.30 (m, 4H), 6.04 (s, 1H), 6.41 (d, J=8 Hz, 1H), 7.81 (d, J=8 Hz, 1H).

MS: [M+H]⁺=316.20

### Example 38

### 1-(6-methyl-3-nitro-2-pyridyl)-4-[(1,3-benzothiazol-6-yl)-methylene]-piperidine

### 1,3-Benzothiazol-6-yl(tributyl)stannane (Compound 38a)

A suspension of 6-bromo-1,3-benzothiazole (100 mg, 0.47 mmol), bis(tributyltin) (304 μl, 0.61 mmol) tetrakis(triphenylphosphine)palladium(0) (27 mg, 0.023 mmol) in anhydrous THF (5 ml) was heated in a microwave oven (Discovery®-CEM) for 10 min at 110° C. The reaction mixture was quenched with water, extracted with EtOAc, dried over Na₂SO₄ and evaporated to dryness in vacuo. The crude was purified by automated flash chromatography (Horizon®-Biotage) eluting with a PE-EtOAc gradient from 10:0 to 9:1 giving the title product as colourless oil. Yield: 39%.

MS: [M+H]⁺=426.41

### 1-(t-butoxycarbonyl)-4-[(1,3-benzothiazol-6-yl)-methylene]-piperidine (Compound 38b)

A suspension of Compound 34a (300 mg, 1.09 mmol), Compound 38a (486 mg, 1.14 mmol) CuI (104 mg, 0.545 mmol), tetrakis(triphenylphosphine)palladium(0) (126 mg, 0.109 mmol) in anhydrous DMF (5 ml) was stirred for 48 hours at r.t. The reaction mixture was quenched with water, extracted with EtOAc, dried over Na₂SO₄ and evaporated to dryness in vacuo. The crude was purified by automated flash chromatography (Horizon®-Biotage) eluting with a PE-EtOAc gradient from 10:0 to 8:2 giving the title product as colourless oil. Yield: 16%.

MS: [M+H]⁺=331.4

### 4-[(1,3-benzothiazol-6-yl)-methylene]-piperidine (Compound 38c)

The title compound was prepared following the procedure described above for Compound 14d, but using Compound 38b instead of Compound 14c. The crude was used in the following step without any further purification. Yield: 100%.

MS: [M+H]⁺=231.3

### 1-(6-methyl-3-nitro-2-pyridyl)-4-[(1,3-benzothiazol-6-yl)-methylene]-piperidine

The title compound was prepared following the procedure described above for the compound of Example 34 but using Compound 38c instead of Compound 34c. Purification was carried out by automated flash chromatography (Horizon®-Biotage) eluting with a PE-EtOAc gradient from 10:0 to 8:2 giving the title product as yellow solid. Yield: 18%.

¹H-NMR (CDCl₃, δ): 2.49 (s, 3H), 2.55-2.60 (m, 2H), 2.65-2.75 (m, 2H), 3.45-3.65 (m, 4H), 6.55 (s, 1H), 6.60 (d, J=8 Hz, 1H), 7.40 (d, J=8 Hz, 1H), 7.82 (s, 1H), 8.05-8.15 (m, 2H), 8.98 (s, 1H)

MS: [M+H]⁺=367.5

### Example 39

### 1-(6-methyl-3-nitro-2-pyridyl)-4-[1-(5-phenyl-1,2,4-oxadiazol-3-yl)-ethylidene]-piperidine

### Diethyl 1-(5-phenyl-1,2,4-oxadiazol-3-yl)-ethylphosphonate (Compound 39a)

Into a suspension of sodium hydride 60% mineral oil dispersion (50 mg, 1.1 mmol) in 5 ml of anhydrous THF cooled at 0-5° C. was dropped a solution of Compound 18a (0.3 g, 1 mmol) in 10 ml of THF anhydrous and the resulting reaction mixture was stirred at r.t. for 30 min. Afterwards, CH₃I (0.126 ml, 2 mmol) was added and stirring continued overnight at r.t. The reaction mixture was quenched with a saturated aqueous solution of NH₄Cl, extracted with EtOAc, dried over Na₂SO₄ and evaporated to dryness in vacuo. The crude, obtained as colourless oil, was used in the next step without further purification.

MS: [M+H]⁺=311.31

### 1-(6-methyl-3-nitro-2-pyridyl)-4-[1-(5-phenyl-1,2,4-oxadiazol-3-yl)-ethylidene]-piperidine

The title product was synthesized following the same procedure reported above for the Compound of Example 1, but using Compound 39a instead of Compound 1b. Purification was carried out by automated flash chromatography (Horizon®-Biotage) eluting with PE-EtOAc 8:2 affording a yellow solid. Yield: 53%.

¹H-NMR (CDCl₃, δ): 2.19 (s, 3H), 2.51 (s, 3H), 2.68-2.74 (m, 2H), 3.00-3.03 (m, 2H), 3.50-3.67 (m, 4H), 6.58-6.60 (m, 1H), 6.62-6.64 (m, 1H), 7.54-7.64 (m, 3H), 8.09-8.19 (m, 3H).

MS: [M+H]⁺=392.43

### Example 40

### 1-(6-methyl-3-nitro-2-pyridyl)-4-{[5-(3-methylphenyl)-1,2,4-oxadiazol-3-yl]-methylene}-piperidine

### Diethyl [5-(3-methylphenyl)-1,2,4-oxadiazol-3-yl]-methylphosphonate (Compound 40a)

The title product was synthesised following the same procedure reported above for Compound 1b, but using 3-chloromethyl-5-m-tolyl-1,2,4-oxadiazole instead of Compound 1a. Usual work-up and purification by automated flash chromatography (Horizon®-Biotage) eluting with CH₂Cl₂-MeOH 95:5 afforded the desired product as colourless oil. Yield: 99%.

MS: [M+H]⁺=311.29

### 1-(6-methyl-3-nitro-2-pyridyl)-4-{[5-(3-methylphenyl)-1,2,4-oxadiazol-3-yl]-methylene}-piperidine

The title product was synthesized following the same procedure reported above for the Compound of Example 1 but using Compound 40a instead of Compound 1b. Purification was carried out by automated flash chromatography (Horizon®-Biotage) eluting with PE-EtOAc 8:2 affording a yellow solid. Yield: 70%.

¹H-NMR (CDCl₃, δ): 2.48 (s, 3H), 2.54 (s, 3H), 2.64-2.66 (m, 2H), 3.24-3.27 (m, 2H), 3.60-3.66 (m, 4H), 6.36 (s, 1H), 6.63-6.65 (m, 1H), 7.43-7.46 (m, 2H), 7.96-7.99 (m, 2H), 8.12-8.14 (m, 1H, 2H).

MS: [M+H]⁺=392.54

### Example 41

### 1-(6-methyl-3-nitro-2-pyridyl)-4-{[5-(3,5-dimethylisoxazol-4-yl)-1,2,4-oxadiazol-3-yl]-methylene}-piperidine

### Diethyl [5-(3,5-dimethylisoxazol-4-yl)-1,2,4-oxadiazol-3-yl]-methylphosphonate (Compound 41a)

The title product was synthesised following the same procedure reported above for Compound 1b, but using 3-chloromethyl-5-(3,5-dimethyl-isoxazol-4-yl)-1,2,4-oxadiazole instead of Compound 1a. Usual work-up and purification by automated flash chromatography (Horizon®-Biotage) eluting with CH₂Cl₂-MeOH 95:5 afforded the desired product as colourless oil. Yield: 86%.

MS: [M+H]⁺=316.32

### 1-(6-methyl-3-nitro-2-pyridyl)-4-{[5-(3,5-dimethylisoxazol-4-yl)-1,2,4-oxadiazol-3-yl]-methylene}-piperidine

The title product was synthesized following the same procedure reported above for the compound of Example 1, but using Compound 41a instead of Compound 1b. Purification was carried out by automated flash chromatography (SP01®-Biotage) eluting with PE-EtOAc 8:2 affording brown oil. Yield: 24%.

¹H-NMR (CDCl₃, δ): 2.51 (s, 3H), 2.61-2.66 (m, 5H), 2.82 (s, 3H), 3.19-3.21 (m, 2H), 3.59-3.64 (m, 4H), 6.35 (s, 1H), 6.63-6.65 (m, 1H), 8.12-8.14.

MS: [M+H]⁺=397.40.

### Example 42

### 1-(6-methyl-3-nitro-2-pyridyl)-4-[(3-isopropyl-1,2,4-oxadiazol-5-yl)-methylene]-piperidine

### Diethyl (3-isopropyl-1,2,4-oxadiazol-5-yl)-methylphosphonate (Compound 42a)

The title compound was prepared following the procedure described above for Compound 37a, but using 5-chloromethyl-3-isopropyl-1,2,4-oxadiazole instead of 5-chloromethyl-3-methyl-1,2,4-oxadiazole. The crude was used in the following step without any further purification. Yield: 100%.

MS: [M+H]⁺=263.3

### 1-(t-butoxycarbonyl)-4-[(3-isopropyl-1,2,4-oxadiazol-5-yl)-methylene]-piperidine (Compound 42b)

The title compound was prepared following the procedure described above for the compound of Example 1, but using Compound 42a instead of Compound 1b and using 1-(t-butoxycarbonyl)-4-oxo-piperidine instead of 1-(6-methyl-3-nitropyridin-2-yl)-4-oxo-piperidine. The crude was used in the following step without any further purification. Yield: 100%.

MS: [M+H]⁺=308.4

### 4-[(3-Isopropyl-1,2,4-oxadiazol-5-yl)methylene]piperidine (Compound 42c)

The title compound was prepared following the procedure described above for Compound 14d, but using Compound 42b instead of Compound 14c. The crude was used in the following step without any further purification. Yield: 100%.

MS: [M+H]⁺=208.4

### 1-(6-methyl-3-nitro-2-pyridyl)-4-[(3-isopropyl-1,2,4-oxadiazol-5-yl)-methylene]-piperidine

The title compound was prepared following the procedure described above for the compound of Example 34, but using Compound 42c instead of Compound 34c. Purification was carried out by automated flash chromatography (Horizon®-Biotage) eluting with a PE-EtOAc gradient from 10:0 to 8:2 giving the title product as yellow solid. Yield: 16.8%.

¹H-NMR (CDCl₃, δ): 1.36 (d, 6H, J=8 Hz), 2.50 (s, 3H), 2.58-2.68 (m, 1H), 3.12 (spt, 2H), 3.15-3.25 (m, 2H), 3.55-3.65 (m, 4H), 6.34 (s, 1H), 6.64 (d, J=8 Hz, 1H), 8.12 (d, J=8 Hz, 1H)

MS: [M+H]⁺=344.4

### Example 43

### 1-(3-nitro-2-pyridyl)-4-[(5-phenyl-1,2,4-oxadiazol-3-yl)-methylene]-piperidine

### 1-(t-butoxycarbonyl)-4-[(5-phenyl-1,2,4-oxadiazol-3-yl)-methylene]-piperidine (Compound 43a)

The title product was synthesized following the same procedure reported above for the Compound of Example 1, but using 1-(t-butoxycarbonyl)-4-oxo-piperidine instead of 1-(6-methyl-3-nitro-pyridin-2-yl)-4-oxo-piperidine. Purification was carried out by automated flash chromatography (SP1®-Biotage) eluting with CHCl₃-1.5 N NH₃ sol. in MeOH 100:0.5 affording a white solid. Yield: 100%.

MS: [M+H]⁺=342.41

### 4-[(5-phenyl-1,2,4-oxadiazol-3-yl)-methylene]-piperidine (Compound 43b)

A solution of Compound 43a (170 mg, 0.498 mmol), and trifluoroacetic acid (0.389 ml, 4.98 mmol) in CHCl₃ (10 ml) was stirred overnight at r.t. After the usual work-up, a colourless oil was obtained (Yield: 95%), used without further purification in the next step.

MS: [M+H]⁺=242.20

### 1-(3-nitro-2-pyridyl)-4-[(5-phenyl-1,2,4-oxadiazol-3-yl)-methylene]-piperidine

The title product was synthesized following the same procedure reported above for the compound of Example 7, but using Compound 43b and 2-chloro-3-nitropyridine. Purification was carried out by automated flash chromatography (SP1®-Biotage) eluting with PE-EtOAc 85:15 affording a yellow solid. Yield: 67%.

¹H-NMR (CDCl₃, δ): 2.64-2.67 (m, 2H), 3.23-3.26 (m, 2H), 3.59-3.66 (m, 4H), 6.37 (s, 1H), 6.79-6.82 (m, 1H), 7.54-7.64 (m, 3H), 8.17-8.21 (m, 3H), 8.38-8.40 (m, 1H).

MS: [M+H]⁺=364.2

### Example 44

### 1-(6-methyl-3-nitro-2-pyridyl-4-{[5-(3-chlorophenyl)-1,2,4-oxadiazol-3-yl]-methylene}-piperidine

### Diethyl 2-amino-2-hydroxyimino-ethylphosphonate (Compound 44a)

A solution of diethyl cyanomethylphosphonate (2 g, 11.3 mmol), NH₂OH.HCl (0.87 g, 12.4 mmol) and Na₂CO₃ in EtOH (100 ml) and water (20 ml) was heated at reflux for 8 h. The reaction mixture was evaporated to dryness in vacuo; repeated EtOH (3×100 ml) addition and evaporation allowed removal of excess of water. Acetone (150 ml) was added and the resulting suspension was stirred for 1 h. The salts were filtered off and the solution was evaporated to give 2 g of the title product as brown oil (88%).

MS: [M+H]⁺=211.21

### Diethyl 2-amino-2-(3-chlorobenzoyloxy-imino)-ethylphosphonate (Compound 44b)

To a solution of Compound 44a (0.18 g, 0.86 mmol) and DIPEA (0.3 ml, 1.3 mmol) in CH₂Cl₂ (5 ml) cooled at 0-5° C. was added 3-chlorobenzoyl chloride (0.14 ml, 1.1 mmol) and the resulting mixture was stirred overnight at r.t. After alkalinization with 1N NaOH and extraction with dichloromethane, the combined extracts were dried over Na₂SO₄ and evaporated to dryness in vacuo. The resultant colourless oil was used in the next step without further purification. Yield (67%)

MS: [M+H]⁺=349.75

### Diethyl [5-(3-chlorophenyl)-1,2,4-oxadiazol-3-yl]-methylphosphonate (Compound 44c)

A suspension of Compound 44b (200 mg, 0.57 mmol) and TBAF on silica gel (capacity approx 1.5 mmol/g (F⁻)) (100 mg) in anhydrous THF (5 ml) was heated in a microwave oven (SmithCreator®-Biotage) for 60 min at 100° C. The reaction mixture was quenched with a saturated aqueous solution of NH₄Cl, extracted with EtOAc, dried over Na₂SO₄ and evaporated to dryness in vacuo. The crude, obtained as colourless oil, was used in the next step without further purification. Yield: 50%

MS: [M+H]⁺=331.70

### 1-(6-methyl-3-nitro-2-pyridyl)-4-{[5-(3-chlorophenyl)-1,2,4-oxadiazol-3-yl]-methylene}-piperidine

The title product was synthesized following the same procedure reported above for the compound of Example 1, but using Compound 44c instead of Compound 1b. Purification was carried out by automated flash chromatography (SP1®-Biotage) eluting with PE-EtOAc 8:2, affording a yellow solid. Yield: 32%.

¹H-NMR (CDCl₃, δ): 2.54 (s, 3H), 2.64-2.67 (m, 2H), 3.23-3.26 (m, 2H), 3.62-3.66 (m, 4H), 6.36 (s, 1H), 6.64-6.66 (m, 1H), 7.49-7.53 (m, 1H), 7.58-7.60 (m, 1H), 8.05-8.07 (m, 1H), 8.12-8.17 (m, 2H).

MS: [M+H]⁺=412.90.

### Example 45

### 1-(2-cyano-3-pyrazinyl)-4-[(5-phenyl-1,2,4-oxa diazol-3-yl)-methylene]-piperidine

The title product was synthesized following the same procedure reported above for the compound of Example 43, but using 3-chloro-2-cyanopyrazine instead of 2-chloro-3-nitropyridine. Purification was carried out by automated flash chromatography (SP1®-Biotage) eluting with PE-EtOAc 85:15, affording a yellow solid. Yield: 90%.

¹H-NMR (CDCl₃, δ): 2.66-2.69 (m, 2H), 3.26-3.29 (m, 2H), 3.96-4.00 (m, 4H), 6.39 (s, 1H), 7.55-7.65 (m, 3H), 8.05 (d, 1H, J=5 Hz), 8.18-8.19 (m, 2H), 8.29 (d, 1H, J=5 Hz).

MS: [M+H]⁺=345.25

### Example 46

### 1-(2-cyano-4-methoxy-3-pyrazinyl)-4-[(5-phenyl-1,2,4-oxadiazol-3-yl)-methylene]-piperidine

The title product was synthesized following the same procedure reported above for the compound of Example 43, but using 3-chloro-2-cyano-4-methoxypyrazine instead of 2-chloro-3-nitropyridine at 100° C. Purification was carried out by automated flash chromatography (SP1®-Biotage) eluting with PE-EtOAc 70:30, affording a yellow solid. Yield: 46%.

¹H-NMR (CDCl₃, δ): 2.63-2.66 (m, 2H), 3.24-3.27 (m, 2H), 3.88-3.92 (m, 4H), 3.99 (s, 3H), 6.35 (s, 1H), 6.38-6.39 (d, 1H, J=5 Hz), 7.54-7.64 (m, 3H), 8.17-8.19 (m, 2H), 8.44 (d, 1H, J=5 Hz).

MS: [M+H]⁺=373.25

### Example 47

### 1-(6-methyl-3-nitro-2-pyridyl)-4-[(3-phenyl-1,2,4-oxadiazol-5-yl)-methylene]-piperidine

### 1-(t-butoxycarbonyl)-4-[(3-phenyl-1,2,4-oxadiazol-5-yl)-methylene]-piperidine (Compound 47a)

A solution of 1-(t-butoxycarbonyl)-4-(2-chloro-2-oxo-ethylidene)-piperidine (501 mg, 1.93 mmol) and N′-hydroxybenzamidine (263 mg, 1.93 mmol) in anhydrous dioxane (10 ml) was stirred at reflux for 3 hours. Molecular sieves 3 Å (100 mg) were added and the mixture stirred at reflux for 6 hours, cooled to r.t., poured into water, extracted with EtOAc, dried (Na₂SO₄) and evaporated to dryness in vacuo. The crude was purified by automated flash chromatography (Horizon®-Biotage) eluting with a PE-EtOAc gradient from 95:5 to 75:25 and yielding 73 mg of the title compound.

¹H-NMR (CDCl₃, δ): 1.52 (s, 9H), 2.47-2.50 (m, 2H), 3.13-3.16 (m, 2H), 3.59-3.62 (m, 4H), 6.38 (s, 1H), 7.50-7.52 (m, 3H), 8.11-8.14 (m, 2H)

MS: [M+H]⁺=342.33

### 4-[(3-phenyl-1,2,4-oxadiazol-5-yl)-methylene]-piperidine (Compound 47b)

A solution of Compound 47a (30 mg, 0.088 mmol) and trifluoroacetic acid (203 μl, 2.64 mmol) in CHCl₃ (4 ml) was stirred at 60° C. for 1 hour, cooled to r.t., rinsed with CHCl₃ washed with 0.5 N NaOH and water, dried (Na₂SO₄) and evaporated to dryness in vacuo. The crude was used for the next step without further purification.

MS: [M+H]⁺=242.12

### 1-(6-methyl-3-nitro-2-pyridyl)-4-[(3-phenyl-1,2,4-oxadiazol-5-yl)-methylene]-piperidine

A mixture of Compound 47b (21 mg, 0.09 mmol), 2-chloro-6-methyl-3-nitropyridine (18 mg, 0.104 mmol) and diisopropylethylamine (30 μl, 0.174 mmol) in N,N-dimethylacetamide (5 ml) was stirred at r.t. for 2 hours. After overnight resting, it was poured into water, extracted with EtOAc, dried (Na₂SO₄) and evaporated to dryness in vacuo. The crude was purified by automated flash chromatography (Horizon®-Biotage) eluting with a PE-EtOAc gradient from 92:8 to 70:30, affording 13 mg of the title compound.

¹H-NMR (CDCl₃, δ): 2.54 (s, 3H) 2.69 (t, J=5.38 Hz, 2H) 3.34 (t, J=5.50 Hz, 2H) 3.65 (t, J=5.87 Hz, 4H) 6.43 (s, 1H) 6.67 (d, J=8.07 Hz, 1H) 7.47-7.57 (m, 3H) 8.14 (m, J=4.80, 4.80, 3.10 Hz, 3H)

MS: [M+H]⁺=378.17

### Example 48

### 1-(3-cyano-2-thienyl)-4-[(5-phenyl-1,2,4-oxadiazol-3-yl)-methylene]-piperidine

A mixture of Compound 43b (85 mg, 0.353 mmol), 2-bromo-3-cyanothiophen (100 mg, 0.532 mmol), Cs₂CO₃ (695 mg, 2.11 mmol), Pd₂(dba)₃ (2.2 mg, 0.0021 mmol), BINAP (26.3 mg, 0.422 mmol) and TEA (0.98 ml, 0.704 mmol) in toluene (3 ml) was reacted at 115° C. for 7 h. After evaporating to dryness in vacuo, the crude was purified by automated flash chromatography (Horizon®-Biotage) eluting with PE-EtOAc 10:1 affording a white solid. Yield: 66%.

¹H-NMR (CDCl₃, δ): 2.66-2.69 (m, 2H), 3.29-3.32 (m, 2H), 3.64-3.70 (m, 4H), 6.37 (s, 1H), 6.52 (d, 1H, J=5 Hz), 6.92 (d, 1H, J=5 Hz), 7.55-7.65 (m, 3H), 8.16-8.18 (m, 2H).

MS: [M+H]⁺=349.4

### Example 49

### 1-(6-methyl-3-nitro-2-pyridyl)-4-[(1-methyl-1H-benzimidazol-2-yl)-methylene]-piperidine

### 2-hydroxymethyl-1-methyl-1H-benzimidazole (Compound 49a)

A suspension of 1-methyl-2-formyl-1H-benzimidazole (500 mg, 3.12 mmol) and NaBH₄ (134 mg, 3.43 mmol) in anhydrous THF (25 ml) was stirred for 24 hours at r.t. The reaction mixture was quenched with water, extracted with EtOAc, dried over Na₂SO₄ and evaporated to dryness in vacuo. The crude was purified by automated flash chromatography (Horizon®-Biotage) eluting with CHCl₃-1.7M NH₃ sol. in MeOH 100:2 giving the title product as white solid. Yield: 50.4%.

MS: [M+H]⁺=163.5

### 2-chloromethyl-1-methyl-1H-benzoimidazole (Compound 49b)

A suspension of Compound 49a (255 mg, 1.57 mmol) and PS-triphenylphosphine resin (loading 2.2 mmol/g) in CCl₄ (15 ml) was refluxed for 3 hours. The resin was filtered and washed and then the organic solvent was evaporated off in vacuo. The crude was used in the following step without any further purification. Yield: 58.9%.

MS: [M+H]⁺=181.3

### diethyl (1-methyl-1H-benzimidazol-2-yl)methylphosphonate (Compound 49c)

The title compound was prepared following the procedure described above for Compound 37a but using Compound 49b instead of 5-(chloromethyl)-3-methyl-1,2,4-oxadiazole. The crude was used in the following step without any further purification. Yield: 100%.

MS: [M+H]⁺=283.3

### 1-(6-methyl-3-nitro-2-pyridyl)-4-[(1-methyl-1H-benzimidazol-2-yl)-methylene]-piperidine

The title compound was prepared following the procedure described above for the compound of Example 1, but using compound 49c instead of compound 1b. Purification was carried out by automated RP chromatography (SP1®-Biotage) eluting with a gradient 20 mMol. aq. NH₄HCO₃—CH₃CN from 5:5 to 10:0. Yield: 40.2%.

¹H-NMR (CDCl₃, δ): 2.49 (s, 3H), 2.60-2.70 (m, 2H), 3.20-3.30 (m, 2H), 3.55-3.65 (m, 4H), 3.80 (s, 3H), 6.35 (s, 1H), 6.61 (d, J=8 Hz, 1H), 7.25-7.40 (m, 3H), 7.75-7.83 (m, 1H), 8.10 (d, J=8 Hz, 1H)

MS: [M+H]⁺=364.5

### Example 50

### 1-(6-methoxy-3-nitro-2-pyridyl)-4-[(1-benzofuran-2-yl)-methylene]-piperidine

### 1-(t-butoxycarbonyl)-4-[(1-benzofuran-2-yl)-methylene]-piperidine (Compound 50a)

The title compound was synthesised following the procedure reported above for Compound 34b but using 2-benzofuranboronic acid instead of benzo[b]thiophene-2-boronic acid. Purification was carried out by automated flash chromatography (SP1®-Biotage) eluting with a gradient PE-EtOAc from 95:5 to 7:3 giving the title product as yellow solid. Yield: 74%. Used in the next step without further characterization.

### 4-[(1-benzofuran-2-yl)-methylene]-piperidine (Compound 50b)

The title product was synthesised as reported for Compound 47b starting from Compound 50a instead of Compound 47a. Used in the next step without further characterization.

### 1-(6-methoxy-3-nitro-2-pyridyl)-4-[(1-benzofuran-2-yl)-methylene]-piperidine

The title compound was synthesised following the procedure reported above for the compound of Example 24 but using Compound 50b instead of Compound 24b. Purification was carried out by automated RP chromatography (SP1®-Biotage) eluting with CH₃CN:H₂O 85:15, giving the title product. Yield: 12.9%.

¹H-NMR (CDCl₃, δ): 2.6 (m, 2H), 3.0-3.1 (m, 2H), 3.5-3.7 (m, 4H), 4.00 (s, 3H), 6.1-6.2 (m, 1H), 6.3 (s, 1H), 6.6 (s, 1H), 7.2-7.3 (m, 2H), 7.45-7.5 (m, 1H), 7.5-7.6 (m, 1H), 8.2-8.3 (m, 1H).

MS: [M+H]⁺=366.38

### Example 51

### 1-(6-methyl-3-nitro-2-pyridyl)-4-{[5-(3-fluorophenyl)-1,2,4-oxadiazol-3-yl]-methylene}-piperidine

### Diethyl 2-amino-2-(3-fluorobenzoyloxy-imino)-ethylphosphonate (Compound 51a)

The title compound was synthesized following the same procedure reported for Compound 44b, but using 3-fluorobenzoyl chloride instead of 3-chlorobenzoyl chloride. Purification was carried out by automated flash chromatography (Horizon®-Biotage) eluting with CH₂Cl₂-MeOH 95:5 affording the title product as colourless oil. Yield: 33%.

MS: [M+H]⁺=333.45

### Diethyl [5-(3-fluorophenyl)-1,2,4-oxadiazol-3-yl]-methylphosphonate (Compound 51b)

The title compound was synthesized following the same procedure reported for Compound 44c, but using Compound 51a instead of Compound 44b. The crude obtained as colourless oil was used in the next step without further purification. Yield 66%

MS: [M+H]⁺=315.30

### 1-(6-methyl-3-nitro-2-pyridyl)-4-{[5-(3-fluorophenyl)-1,2,4-oxadiazol-3-yl]-methylene}-piperidine

The title product was synthesized following the same procedure reported above for the compound of Example 1 but using Compound 51b instead of Compound 1b. Purification was carried out by automated flash chromatography (SP1®-Biotage) eluting with a PE-EtOAc gradient from 95:5 to 7:3 giving the title product as yellow solid. Yield: 38%.

¹H-NMR (CDCl₃, δ): 2.50 (s, 3H), 2.62-2.65 (m, 2H), 3.21-3.24 (m, 2H), 3.59-3.63 (m, 4H), 6.35 (s, 1H), 6.62-6.64 (m, 1H), 7.29-7.32 (m, 1H), 7.54-7.55 (m, 1H) 7.86-7.88 (m, 1H), 7.96-7.98 (m, 1H), 8.10-8.12 (m, 1H).

MS: [M+H]⁺=396.22

### Example 52

### 1-(6-methyl-3-nitro-2-pyridyl)-4-[(3-methyl-isoxazol-5-yl)-methylene]-piperidine

### 1-(t-butoxycarbonyl)-4-[(3-methyl-isoxazol-5-yl)-methylene]-piperidine (Compound 52a)

To a solution of trimethyl[(3-methyl-isoxazol-5-yl)-methyl]-silane (500 mg, 2.95 mmol, prepared as described in Tetrahedron Letter 1981, 22, 3699) in anhydrous THF (15 ml) stirred at −78° C., under nitrogen atmosphere, was added butyl lithium (2.5M sol. in hexane, 1.2 ml, 2.95 mmol). After 2 h, a solution of 1-(t-butoxycarbonyl)-4-oxo-piperidine (588 mg, 2.95 mmol) in THF (5 ml) was dropped and the resulting mixture was overnight at r.t. The reaction mixture was quenched with a saturated aqueous solution of NH₄Cl, extracted with EtOAc, dried over Na₂SO₄ and evaporated to dryness in vacuo. Purification was carried out by automated flash chromatography (Horizon®-Biotage) eluting with PE-EtOAc 8:2 affording a colourless oil. Yield: 93%.

MS: [M+H]⁺=279.88

### 4-[(3-methyl-isoxazol-5-yl)-methylene]-piperidine (Compound 52b)

The title compound was prepared following the procedure reported above for Compound 21b, but using Compound 52a instead of Compound 21a. The crude, obtained as colourless oil, was used in the next step without further purification.

MS: [M+H]⁺=179.78

### 1-(6-methyl-3-nitro-2-pyridyl)-4-[(3-methyl-isoxazol-5-yl)-methylene]-piperidine

The title product was synthesised following the same procedure reported above for the compound of Example 7, but using Compound 52b instead of Compound 7a. Purification was carried out by automated flash chromatography (Horizon®-Biotage) eluting with PE-EtOAc 9:1, giving the title product as yellow solid. Yield: 37%.

¹H-NMR (CDCl₃, δ): 2.32 (s, 3H), 2.56 (s, 3H), 2.55-2.58 (m, 2H), 2.90-2.92 (m, 2H), 3.53-3.60 (m, 4H), 5.97 (s, 1H), 6.22 (s, 1H), 6.61-6.63 (m, 1H), 8.09-8.11 (m, 1H).

MS: [M+H]⁺=315.52

### Example 53

### 1-(6-methyl-3-nitro-2-pyridyl)-4-[(5-methyl-1,3,4-oxadiazol-2-yl)-methylene]-piperidine

### Diethyl (5-methyl-1,3,4-oxadiazol-2-yl)-methylphosphonate (Compound 53a)

The title compound was prepared following the procedure described above for Compound 37a, but using 2-chloromethyl-5-methyl-1,3,4-oxadiazole instead of 5-chloromethyl-3-methyl-1,2,4-oxadiazole. The crude was used in the following step without any further purification. Yield: 100%.

MS: [M+H]⁺=235.2

### 1-(6-methyl-3-nitro-2-pyridyl)-4-[(5-methyl-1,3,4-oxadiazol-2-yl)-methylene]-piperidine

The title compound was prepared following the procedure described above for the compound of Example 1, but using Compound 53a instead of Compound 1b. Purification was carried out by automated flash chromatography (SP1®-Biotage) eluting with a gradient PE-EtOAc from 75:15 to 0:100 giving the title product as a yellow solid. Yield: 32.2%.

¹H-NMR (CDCl₃, δ): 2.49 (s, 3H), 2.56 (s, 3H), 2.55-2.65 (m, 2H), 3.05-3.15 (m, 2H), 3.50-3.65 (m, 4H), 6.23 (s, 1H), 6.63 (d, J=8 Hz, 1H), 8.11 (d, J=8 Hz, 1H)

MS: [M+H]⁺=316.4

### Example 54

### 1-(6-methyl-3-nitro-2-pyridyl)-4-[(5-phenyl-2H-tetrazol-2-yl)-methylene]-piperidine

### 1-(t-butoxycarbonyl)-4-[(5-phenyl-2H-tetrazol-2-yl)-methylene]-piperidine (Compound 54a)

The title product was synthesised following the same procedure reported above for the Compound 52a, but using 5-phenyl-2-trimethylsilanylmethyl-2H-tetrazole (prepared as described in J. Chem Soc Perk Trans 1, 1991, 2, 323) instead of trimethyl[(3-methyl-isoxazol-5-yl)-methyl]-silane. The crude was used for the next step without further purification. Yield: 70%.

MS: [M+H]⁺=342.23

### 4-[(5-phenyl-2H-tetrazol-2-yl)-methylene]-piperidine (Compound 54b)

The title compound was prepared following the procedure reported above for Compound 21b, but using Compound 54a instead of Compound 21a. The crude, obtained as colourless oil, was used in the next step without further purification.

MS: [M+H]⁺=242.33

### 1-(6-methyl-3-nitro-2-pyridyl)-4-[(5-phenyl-2H-tetrazol-2-yl)-methylene]-piperidine

The title product was synthesised following the same procedure reported above for the compound of Example 7 but using Compound 54b instead of Compound 7a. Purification was carried out by automated flash chromatography (Horizon®-Biotage) eluting with PE-EtOAc 9:1 and giving the title product as a yellow solid. Yield: 90%.

¹H-NMR (CDCl₃, δ): 2.52 (s, 3H), 2.64-2.67 (m, 2H), 3.13-3.16 (m, 2H), 3.61-3.66 (m, 4H), 6.65-6.67 (m, 1H), 7.50-7.55 (m, 3H), 8.12-8.14 (m, 1H), 8.20-8.22 (m, 2H).

MS: [M+H]⁺=378.52

### Example 55

### 1-(6-methyl-3-nitro-2-pyridyl)-4-{[5-(6-methyl-2-pyridyl)-1,2,4-oxadiazol-3-yl]-methylene}-piperidine

### Diethyl 2-amino-2-(6-methylpyridin-2-ylcarbonyloxy-imino)-ethylphosphonate (Compound 55a)

The title compound was synthesized following the same procedure reported for the Compound 44b, but using 6-methylpyridine-2-carbonyl chloride instead of 3-chlorobenzoyl chloride. The crude, obtained as brown oil, was used in the next step without further purification. Yield 53%

MS: [M+H]⁺=330.30.

### Diethyl [5-(6-methyl-2-pyridyl)-1,2,4-oxadiazol-3-yl]-methylphosphonate (Compound 55b)

The title compound was synthesized following the same procedure reported for the Compound 44c, but using Compound 55a instead of Compound 44b. Purification was carried out by automated flash chromatography (Horizon®-Biotage) eluting with CH₂Cl₂-MeOH 95:5 affording a brown oil. Yield: 21%.

MS: [M+H]⁺=312.41

### 1-(6-methyl-3-nitro-2-pyridyl)-4-{[5-(6-methyl-2-pyridyl)-1,2,4-oxadiazol-3-yl]-methylene}-piperidine

The title product was synthesized following the same procedure reported above for the compound of Example 1 but using Compound 55b instead of Compound 1b. Purification was carried out by automated flash chromatography (SP1®-Biotage) eluting with a PE-EtOAc gradient from 95:5 to 7:3 giving the title product as a yellow solid. Yield: 63%.

¹H-NMR (CDCl₃, δ): 2.51 (s, 3H), 2.62-2.65 (m, 2H), 2.73 (s, 3H), 3.21-3.23 (m, 2H), 3.58-3.64 (m, 4H), 6.39 (s, 1H), 6.62-6.64 (m, 1H), 7.39-7.41 (m, 1H), 7.79-7.83 (m, 1H), 8.06-8.13 (m, 2H).

MS: [M+H]⁺=393.42

### Example 56

### 1-(2-cyano-3-pyrazinyl)-4-{[5-(3-thienyl)-1,2,4-oxadiazol-3-yl]-methylene}-piperidine

### 2-cyano-3-(4-oxopiperidino)-pyrazine (Compound 56a)

A mixture of 2-cyano-3-chloropyrazine (500 mg, 3.4 mmol), piperidin-4-one hydrochloride monohydrate (1.33 g, 8.5 mmol) and TEA (1.8 ml, 10.3 mmol) in toluene (20 ml) was reacted at 80° C. for 8 h. After diluting with H₂O and EtOAc, the organic phase was evaporated to dryness in vacuo. The crude was purified by automated flash chromatography (Horizon®-Biotage) eluting with PE-EtOAc 80:20, affording a yellow solid. Yield: 69%.

MS: [M+H]⁺=203.15

### 1-(2-cyano-3-pyrazinyl)-4-{[5-(3-thienyl)-1,2,4-oxadiazol-3-yl]-methylene}-piperidine

The title product was synthesized following the same procedure reported above for the compound of Example 1, but using Compounds 29 and 56a. Purification was carried out by automated flash chromatography (SP1®-Biotage) eluting with PE-EtOAc 8:2 affording a white solid. Yield: 86%.

¹H-NMR (CDCl₃, δ): 2.65-2.68 (m, 2H), 3.23-3.26 (m, 2H), 3.95-3.99 (m, 4H), 6.37 (s, 1H), 7.50 (d, 1H), 7.71 (d, 1H), 8.05 (d, 1H), 8.25-8.26 (m, 1H), 8.30 (d, 1H).

MS: [M+H]⁺=351.41.

### Example 57

### 1-(6-methyl-3-nitro-2-pyridyl)-4-{[(2-(2-furyl)-1,3-thiazol-4-yl]-methylene}-piperidine

### Diethyl [2-(2-furyl)-1,3-thiazol-4-yl]-methylphosphonate (Compound 57a)

The title product was synthesized following the same procedure reported above for the Compound 1b of Example 1, but using 4-chloromethyl-2-(2-furyl)-thiazole instead of Compound 1a. Purification was carried out by automated flash chromatography (SP1®-Biotage) using a RP18 column and eluting with a 0.02M solution of (NH₄)HCO₃-MeCN gradient from 10:0 to 4:6 affording an oil. Yield: 47%.

MS: [M+H]⁺=302.3.

### 1-(6-methyl-3-nitro-2-pyridyl)-4-{[(2-(2-furyl)-1,3-thiazol-4-yl]-methylene}-piperidine

The title product was synthesized following the same procedure reported above for the Compound of Example 1, but using Compound 57a instead of 1b. Purification was carried out by automated flash chromatography (SP1®-Biotage) eluting with PE-EtOAc 8:2 affording a yellow oil. Yield: 30%.

¹H-NMR (CDCl₃, δ): 2.49 (s, 3H), 2.55-2.58 (m, 2H), 3.07-3.10 (m, 2H), 6.42 (s, 1H), 6.55-6.60 (m, 2H), 7.01-7.04 (m, 2H), 7.53 (s, 1H), 8.09 (d, 1H, J=8 Hz).

MS: [M+H]⁺=383.3.

### Example 58

### 1-(6-methyl-3-nitro-2-pyridyl)-4-[(2-phenyl-1,3-thiazol-4-yl)-methylene]-piperidine

### Diethyl (2-phenyl-1,3-thiazol-4-yl)-methylphosphonate (Compound 58a)

The title product was synthesized following the same procedure reported above for Compound 1b, but using 4-chloromethyl-2-phenylthiazole instead of Compound 1a. Purification was carried out by automated chromatography (SP1®-Biotage) using a RP18 column and eluting with a 0.02M solution of (NH₄)HCO₃-MeCN gradient from 9:1 to 2:8 affording an oil. Yield: 76%.

MS: [M+H]⁺=312.3

### 1-(t-butoxycarbonyl)-4-[(2-phenyl-1,3-thiazol-4-yl)-methylene]-piperidine (Compound 58b)

The title product was synthesized following the same procedure reported above for the compound of Example 1, but using 1-(t-butoxycarbonyl)-4-oxo-piperidine instead of 1-(6-methyl-3-nitropyridin-2-yl)-4-oxo-piperidine. Purification was carried out by automated flash chromatography (SP1®-Biotage) eluting with PE-EtOAc 8:2, affording a white solid. Yield: 45%.

MS: [M+H]⁺=357.41

### 4-[(2-phenyl-1,3-thiazol-4-yl)-methylene]-piperidine (Compound 58c)

A solution of Compound 58b (50 mg, 0.14 mmol) and trifluoroacetic acid (0.22 ml, 2.82 mmol) in CHCl₃ (5 ml) was stirred overnight at r.t. After the usual work-up, a colourless oil was obtained (Yield: 97%.), used without further purification in the next step.

MS: [M+H]⁺=257.3

### 1-(6-methyl-3-nitro-2-pyridyl)-4-[(2-phenyl-1,3-thiazol-4-yl)-methylene]-piperidine

The title product was synthesized following the same procedure reported above for the compound of Example 7, but using Compound 58c and 2-chloro-6-methyl-3-nitropyridine. Purification was carried out by automated flash chromatography (SP1®-Biotage) eluting with PE-EtOAc 92:8 and affording a yellow solid. Yield: 84%.

¹H-NMR (CDCl₃, δ): 2.49 (s, 3H), 2.57-2.59 (m, 2H), 3.19-3.22 (m, 2H), 3.57-3.62 (m, 4H), 6.42 (s, 1H), 6.59 (d, 1H, J=8 Hz), 7.05 (s, 1H), 7.44-7.50 (m, 3H), 7.98-8.00 (m, 2H), 8.10 (d, 1H, J=8 Hz).

MS: [M+H]⁺=393.4

### Example 59

### 1-(6-methyl-3-nitro-2-pyridyl)-4-[(4-fluoro-1-benzofuran-2-yl)-methylene]-piperidine

The title compound was prepared following the procedure described for the Compound of Example 24, but using 3-fluoro-2-iodophenol instead of 2-iodophenol. Purification was carried out by automated flash chromatography (SP1®-Biotage) eluting with a PE-EtOAc gradient from 10:0 to 7:3 affording an orange oil. Yield: 13%.

¹H-NMR (CDCl₃, δ): 2.5 (m, 1H), 2.6 (m, 2H), 3.0-3.1 (m, 2H), 3.5-3.65 (m, 4H), 6.2-6.3 (m, 1H), 6.6 (m, 2H), 6.9 (m, 1H), 7.2-7.25 (m, 1H), 7.25-7.3 (m, 1H), 8.1 (m, 1H). MS: [M+H]+=MS: [M+H]⁺=368.38

### Example 60

### 1-(2-cyano-3-pyrazinyl)-4-[(1-benzofuran-2-yl)-methylene]-piperidine

### 1-(2-cyano-3-pyrazinyl)-4-(3-trimethylsilyl-prop-2-ynylidene)-piperidine (Compound 60a)

The title product was synthesized following the same procedure reported above for the compound of Example 1, but reacting Compound 56a and Compound 3a. Purification was carried out by automated flash chromatography (SP1®-Biotage) eluting with a PE-EtOAc gradient from 100:0.5 to 9:1 affording a yellow oil. Yield: 68%.

MS: [M+H]⁺=297.4.

### 1-(2-cyano-3-pyrazinyl)-4-[(1-benzofuran-2-yl)-methylene]-piperidine

A mixture of Compound 60a (100 mg, 0.337 mmol), 2-iodophenol (80 mg, 0.356 mmol), tetrakis(triphenylphosphine)palladium(0) (21 mg, 0.018 mmol), tetrabutylammonium fluoride.H₂O (91 mg, 0.341 mmol), sodium acetate trihydrate (92 mg, 0.676 mmol) and DMF (3 ml) degassed by flowing nitrogen, was heated in a microwave oven (Discovery®-CEM) at 120° C. for 5 min. Quench with water and extraction with EtOAc afforded a residue, which was purified by automated flash chromatography (SP1®-Biotage) eluting with PE-EtOAc 9:1 affording a yellow oil. Yield: 35%.

¹H-NMR (CDCl₃, δ): 2.61-2.64 (m, 2H), 3.11-3.14 (m, 2H), 3.95-3.99 (m, 4H), 6.31 (s, 1H), 6.58 (s, 1H), 7.21-7.34 (m, 2H), 7.47 (d, 1H, J=8 Hz), 7.55 (d, 1H, J=8 Hz), 8.03 (d, 1H, J=4 Hz), 8.28 (d, 1H, J=4 Hz).

MS: [M+H]⁺=317.4.

### Example 61

### 1-(6-methyl-3-nitro-2-pyridyl)-3-[(5-phenyl-1,2,4-oxadiazol-3-yl)-methylene]-azetidine

### 1-(t-butoxycarbonyl)-3-[(5-phenyl-1,2,4-oxadiazol-3-yl)-methylene]-azetidine (Compound 61a)

The title product was synthesised following the same procedure reported above for the compound of Example 1, but using Compound 18a instead of Compound 1b and 1-(t-butoxycarbonyl)-4-oxo-azetidine instead of 1-(6-methyl-3-nitro-2-pyridyl)-4-oxo-piperidine. Purification was carried out by automated flash chromatography (Horizon®-Biotage) eluting with a PE-EtOAc gradient from 95:5 to 8:2, affording the title compound. Yield: 73.6%.

MS: [M+H]⁺=314.43

### 3-[(5-phenyl-1,2,4-oxadiazol-3-yl)-methylene]-azetidine (Compound 61b)

Prepared following the procedure reported above for Compound 21b, but using Compound 61a instead of Compound 21a. Obtained as colourless oil and used in the next step without further purification.

MS: [M+H]⁺=214.32

### 1-(6-methyl-3-nitro-2-pyridyl)-3-[(5-phenyl-1,2,4-oxadiazol-3-yl)-methylene]-azetidine

The title product was synthesised following the same procedure reported above for the compound of Example 7, but using Compound 61b instead of Compound 7a. Purification was carried out by automated flash chromatography (Horizon®-Biotage) eluting with a PE-EtOAc gradient from 95:5 to 7:3, affording the title compound as yellow solid. Yield: 32%.

¹H-NMR (CDCl₃, δ): 2.54 (s, 3H) 4.81-5.10 (m, 2H) 5.10-5.37 (m, 2H) 6.47 (t, J=2.32 Hz, 1H) 6.64 (d, J=8.31 Hz, 1H) 7.53-7.60 (m, 2H) 7.62 (d, J=7.34 Hz, 1H) 8.21 (d, J=8.31 Hz, 1H) 8.18 (d, J=7.09 Hz, 2H)

MS: [M+H]⁺=350.23

### Example 62

### 1-(6-methyl-3-nitro-2-pyridyl)-(4E,Z)-4-[(5-phenyl-1,2,4-oxadiazol-3-yl)-methylene]-perhydroazepine

### 1-(t-butoxycarbonyl)-(4E,Z)-4-[(5-phenyl-1,2,4-oxadiazol-3-yl)-methylene]-perhydroazepine (Compound 62a)

The title product was synthesised following the same procedure reported above for the compound of Example 1, but using Compound 18a instead of Compound 1b and 1-(t-butoxycarbonyl)-4-oxo-perhydro azepine instead of 1-(6-methyl-3-nitro-2-pyridyl)-4-oxo-piperidine. Purification was carried out by automated flash chromatography (Horizon®-Biotage) eluting with a PE-EtOAc gradient from 8:2 to 1:1, affording the title compound. Yield: 77.6%.

MS: [M+H]⁺=356.44

### (4E,Z)-4-[(5-phenyl-1,2,4-oxadiazol-3-yl)-methylene]-perhydroazepine (Compound 62b)

Prepared following the procedure reported above for Compound 21b, but using Compound 62a instead Compound 21a. Obtained as colourless oil, used in the next step without further purification.

MS: [M+H]⁺=256.32

### 1-(6-methyl-3-nitro-2-pyridyl)-(4E,Z)-4-[(5-phenyl-1,2,4-oxadiazol-3-yl)-methylene]-perhydroazepine

The title product was synthesised following the same procedure reported above for the compound of Example 7, but using Compound 62b instead of Compound 7a. Purification was carried out by automated flash chromatography (SP1®-Biotage) eluting with a PE-EtOAc gradient from 95:5 to 7:3, affording the title compound as yellow solid. Yield: 92%. The title compound was characterized by ¹H-NMR as a 1:1 mixture of E and Z isomers.

¹H-NMR (CDCl₃, δ): 2.43 (s, 3H), 2.67-2.80 (m, 2H), 3.22-3.26 (m, 2H), 3.39-3.73 (m, 6H), 6.25 (s, 1H), 6.62-6.64 (m, 1H), 7.52-7.62 (m, 5H), 7.96-7.98 (m, 1H), 8.10-8.12 (m, 1H).

MS: [M+H]⁺=392.44

### Example 63

### 1-(6-methyl-3-nitro-2-pyridyl)-4-{[5-(3-methylphenyl)-2H-tetrazol-2-yl]-methylene}-piperidine

### 2-Methyl-5-(3-methylphenyl)-2H-tetrazole (Compound 63a)

A solution of iodomethane (0.77 ml, 12.5 mmol) in acetone (20 ml) was dropped into a solution of 5-(3-methylphenyl)-1H-tetrazole (2 g, 12.5 mmol) and NaOH (1 g, 25 mmol) in water (10 ml). The resulting mixture was stirred at reflux for 6 hours. Afterwards, it was evaporated to dryness and taken up with EtOAc and H₂O. The organic layer was separated, dried over Na₂SO₄ and evaporated to dryness in vacuo. Purification was carried out by automated flash chromatography (SP1®-Biotage) eluting with a PE-EtOAc gradient from 95:5 to 6:4, affording the title compound as white solid. Yield: 57.6%.

MS: [M+H]⁺=175.44

### 2-trimethylsilylmethyl-5-(3-methylphenyl)-2H-tetrazole (Compound 63b)

t-Butyllithium (1.7 M in pentane, 2.42 ml, 4.11 mmol) was dropped into a cooled solution of Compound 63a (0.6 g, 3.44 mmol) in THF anhydrous (15 ml) stirred at −78° C. under a nitrogen atmosphere. After 1 hour, trimethylchlorosilane (0.52 ml, 4.12 mmol) was added, and the temperature was allowed to rise to r.t overnight. The reaction mixture was quenched with a saturated aqueous solution of NH₄Cl, extracted with EtOAc, dried over Na₂SO₄ and evaporated to dryness in vacuo. Thr crude product was used for the next step without further purification.

MS: [M+H]⁺=247.40

### 1-(6-methyl-3-nitro-2-pyridyl)-4-{[5-(3-methylphenyl)-2H-tetrazol-2-yl]-methylene}-piperidine

The title product was synthesized following the same procedure reported above for the Compound 52a, but using Compound 63b instead of trimethyl[(3-methyl-isoxazol-5-yl)-methyl]-silane. and 1-(6-methyl-3-nitro-2-pyridyl)-4-oxo-piperidine instead of 1-(t-butoxycarbonyl)-4-oxo-piperidine. Purification was carried out by automated flash chromatography (SP1®-Biotage) eluting with a PE-EtOAc gradient from 95:5 to 7:3 and affording the title compound as yellow solid. Yield: 10%.

¹H-NMR (CDCl₃, δ): 2.47 (s. 3H), 2.51 (s, 3H), 2.63-2.66 (m, 2H), 3.13-3.15 (m, 2H), 3.61-3.66 (m, 4H), 6.65-6.67 (m, 1H), 7.28 (s, 1H), 7.40-7.44 (m, 2H), 7.99-8.03 (m, 2H), 8.12-8.14 (m, 1H).

MS: [M+H]⁺=392.44

### Example 64

### 1-(6-methyl-3-nitro-2-pyridyl)-(3Z)-3-[(5-phenyl-1,2,4-oxadiazol-3-yl)-methylene]-pyrrolidine

### 1-(t-butoxycarbonyl)-(3Z)-3-[(5-phenyl-1,2,4-oxadiazol-3-yl)-methylene]-pyrrolidine (Compound 64a)

The title product was synthesised following the same procedure reported above for the compound of Example 1, but using Compound 18a instead of Compound 1b and 1-(t-butoxycarbonyl)-3-oxo-pyrrolidine instead of 1-(6-methyl-3-nitro-2-pyridyl)-4-oxo-piperidine. Purification was carried out by automated flash chromatography (SP1®-Biotage) eluting with a PE-EtOAc gradient from 8:2 to 1:1 affording the title compound. Yield: 38%. The E enantiomer (Compound 65a) was also isolated (36% yield).

MS: [M+H]⁺=328.27

### (3Z)-3-[(5-phenyl-1,2,4-oxadiazol-3-yl)-methylene]-pyrrolidine (Compound 64b)

The title compound was prepared following the procedure reported above for Compound 21b, but using Compound 64a instead of Compound 21a. It was obtained as colourless oil and used in the next step without further purification.

MS: [M+H]⁺=228.32

### 1-(6-methyl-3-nitro-2-pyridyl)-(3Z)-3-[(5-phenyl-1,2,4-oxadiazol-3-yl)-methylene]-pyrrolidine

The title product was synthesised following the same procedure reported above for the compound of Example 7, but using Compound 64b instead of Compound 7a. Purification was carried out by automated flash chromatography (SP1®-Biotage) eluting with a PE-EtOAc gradient from 95:5 to 7:3, affording the title compound as yellow solid. Yield: 76%.

¹H-NMR (CDCl₃, δ): 2.53 (s, 3H) 3.36 (t, J=7.34 Hz, 2H) 3.76 (t, J=7.34 Hz, 2H) 4.46 (s, 2H) 6.52 (t, J=2.20 Hz, 1H) 6.61 (d, J=8.07 Hz, 1H) 7.52-7.60 (m, 2H) 7.60-7.66 (m, 1H) 8.07 (d, J=8.07 Hz, 1H) 8.14-8.23 (m, 2H)

MS: [M+H]⁺=364.38

### Example 65

### 1-(6-methyl-3-nitro-2-pyridyl)-(3E)-3-[(5-phenyl-1,2,4-oxa diazol-3-yl)-methylene]-pyrrolidine

### 1-(t-butoxycarbonyl)-(3E)-3-[(5-phenyl-1,2,4-oxadiazol-3-yl)-methylene]-pyrrolidine (Compound 65a)

The title compound was isolated during the purification of Compound 64a.

MS: [M+H]⁺=328.27

### (3E)-3-[(5-phenyl-1,2,4-oxadiazol-3-yl)-methylene]-pyrrolidine (Compound 65b)

Prepared following the procedure reported above for Compound 21b, but using Compound 65a instead Compound 21a. Obtained as colourless oil and used in the next step without further purification.

MS: [M+H]⁺=228.32

### 1-(6-methyl-3-nitro-2-pyridyl)-(3E)-3-[(5-phenyl-1,2,4-oxadiazol-3-yl)-methylene]-pyrrolidine

The title product was synthesised following the same procedure reported above for the Compound of Example 7, but using Compound 65b instead of Compound 7a. Purification was carried out by automated flash chromatography (SP1®-Biotage), eluting with a PE-EtOAc gradient from 95:5 to 7:3 affording the title compound as yellow solid. Yield: 70%.

¹H-NMR (CDCl₃, δ): 2.56 (s, 3H) 2.93-3.07 (m, 2H) 3.83 (t, J=7.46 Hz, 2H) 4.69 (s, 2H) 6.52 (t, J=2.08 Hz, 1H) 6.60 (d, J=8.31 Hz, 1H) 7.52-7.66 (m, 3H) 8.06 (d, J=8.07 Hz, 1H) 8.14-8.26 (m, 2H)

MS: [M+H]⁺=364.38

### Example 66

### 1-(6-methyl-3-nitro-2-pyridyl)-4-(thieno[3,2-b]benzothien-2-yl)-methylene}-piperidine

### 2-hydroxymethyl-thieno[3,2-b]benzothiophen (Compound 66a)

Into a suspension of 2-carboxy-thieno[3,2-b]-benzothiophen (535 mg, 2.28 mmol) in 5 ml of anhydrous THF was added dropwise at 0° C. over 20 minutes a solution of BH₃.Me₂S (1M, 2.51 ml, 2.51 mmol). The reaction mixture was stirred at 0° for 15 min., then it was allowed to warm up to r.t. and was stirred at this temperature for 16 h. The reaction was quenched with methanol (6 ml) and the clear solution was concentrated in vacuo. Water was added and the product was extracted with CH₂Cl₂. NaOH was added and the mixture was stirred vigorously. The combined organic layers were washed with brine, dried on Na₂SO₄ and evaporated to dryness in vacuo to afford 455 mg (90.6%) of the title product as white solid, enough pure to be used in the following step without any further purification.

MS: [M+NH₄]⁺=238.49

### 2-chloromethyl-thieno[3,2-b]benzothiophen (Compound 66b)

To a suspension of PS-triphenylphosphine resin (1.136 g., 2.5 mmol, resin's loading=2.2 mmol/g) in 12 ml of CCl₄ was added Compound 66a (275 mg., 1.25 mmol) and the reaction was heated at reflux for 3 h. Afterwards, the solution was filtered and concentrated to dryness to give 278 mg. (86%) of the title product (yellow solid). This crude was used in the following step without any further purification.

### Diethyl thieno[3,2-b]benzothien-2-yl-methylphosphonate (Compound 66c)

To a solution of 278 mg. of Compound 66b (1.16 mmol) in 5 ml of xylene was added triethyl phosphite (5 ml) and the reaction was heated at 160° C. for 24 h. The solvent and the excess of triethyl phosphite were distilled at 90° C. at reduced pressure (1 mbar) to give a crude that was purified by automated flash chromatography (Horizon®-Biotage) eluting with CHCl₃, giving 312 mg (79%) of the title product.

MS: [M+H]⁺=341.41

### 1-(6-methyl-3-nitro-2-pyridyl)-4-(thieno[3,2-b]benzothien-2-yl)-methylene}-piperidine

The title compound was prepared following the procedure reported for the Compound of Example 1, but using Compound 66c instead of Compound 1b. To obtain the full conversion of the starting material, the reaction mixture was heated at reflux for 2 h. After the usual work-up procedure, the crude was purified by automated flash chromatography (Horizon®-Biotage) eluting with CHCl₃, affording the title compound as a yellow solid (55%).

¹H-NMR (CDCl₃, δ): 2.53 (s, 3H), 2.62 (t, J=5.62 Hz, 2H), 2.93 (t, J=5.62 Hz, 2H), 3.59 (t, J=5.87 Hz, 2H), 3.65 (t, J=5.75 Hz, 2H), 6.57 (s, 1H), 6.62 (d, J=8.31 Hz, 1H), 7.16 (s, 1H), 7.36 (d, J=7.34 Hz, 1H), 7.39-7.48 (m, 1H), 7.81 (d, J=7.83 Hz, 1H), 7.85 (d, J=8.07 Hz, 1H), 8.12 (d, J=8.31 Hz, 1H).

MS: [M+H]⁺=422.54

### Example 67

### 1-(6-methyl-3-nitro-2-pyridyl)-4-[(7-fluoro-1-benzofuran-2-yl)-methylene]-piperidine

The title compound was prepared following the procedure described for the Compound of Example 24, but using 2-fluoro-6-iodophenol instead of 2-iodophenol. Purification was carried out by automated flash chromatography (SP1®-Biotage) eluting with a PE-EtOAc gradient from 10:0 to 8:2 followed by preparative LC-MS, affording the title product. Yield: 12%.

¹H-NMR (CDCl₃, δ): 2.51 (s, 3H), 2.60 (t, J=5.62 Hz, 2H), 3.07 (t, J=5.75 Hz, 2H), 3.53-3.72 (m, 4H), 6.29 (s, 1H), 6.53-6.60 (m, 1H), 6.61 (d, J=8.31 Hz, 1H), 6.95-7.05 (m, 1H), 7.14 (td, J=7.82, 4.40 Hz, 1H), 7.23-7.34 (m, 1H), 8.11 (d, J=8.31 Hz, 1H).

MS: [M+H]⁺=368.38

### Example 68

### 1-(6-methyl-3-nitro-2-pyridyl)-4-{[1-(t-butoxycarbonyl)-6-methoxy-2-indolyl]-methylene}-piperidine

### 1-(t-butoxycarbonyl)-4-{[1-(t-butoxycarbonyl)-6-methoxy-2-indolyl]-methylene}-piperidine (Compound 68a)

To a solution of Compound 34a (0.15 g, 0.53 mmol) and dichloro-bis[dicyclohexyl-[2-(2,6-dimethoxyphenyl)phenyl]phosphoranyl]palladium (0.031 g, 0.03 mmol) in toluene (4.5 ml) was added 1-(t-butoxycarbonyl)-6-methoxy-2-indolylboronic acid (0.24 g, 0.82 mmol) and potassium phosphate (0.25 g, 1.19 mmol). The reaction mixture was reacted in a microwave oven (CEM) for 600 sec at 120° C. After the usual work-up, the residue was purified by automated flash chromatography (SP1®-Biotage) eluting with a PE-EtOAc gradient from 10:0 to 8:2 affording the title product. Yield: 91.5%.

### 4-{[1-(t-butoxycarbonyl)-6-methoxy-2-indolyl]-methylene}-piperidine (Compound 68b)

The title product was synthesised as reported for Compound 47b starting from Compound 68a instead of Compound 47a and heating at 65° C. Used in the next step without further characterization.

### 1-(6-methyl-3-nitro-2-pyridyl)-4-{[1-(t-butoxycarbonyl)-6-methoxy-2-indolyl]-methylene}-piperidine

The title product was synthesised as reported for the Compound of Example 47 starting from Compound 68b instead of Compound 47b. After the work-up, the crude was purified by automated flash chromatography (SP1®-Biotage) eluting with a PE-EtOAc gradient from 92:8 to 70:30, affording the title compound as yellow oil. Yield: 76%.

¹H-NMR (CDCl₃, δ): 1.69 (s, 9H), 2.49 (s, 3H), 2.57 (t, J=5.50 Hz, 2H), 2.73 (t, J=5.50 Hz, 2H), 3.47-3.54 (m, 2H), 3.54-3.61 (m, 2H), 3.90 (s, 3H), 6.35 (s, 1H), 6.55-6.64 (m, 2H), 6.88 (dd, J=8.56, 2.20 Hz, 1H), 7.38 (d, J=8.56 Hz, 1H), 7.77 (d, J=2.20 Hz, 1H), 8.09 (d, J=8.07 Hz, 1H)

MS: [M+H]⁺=479.54

### Example 69

### 1-(6-methyl-3-nitro-2-pyridyl)-4-[(6-fluoro-2-indolyl)-methylene]-piperidine

### 1-(t-butoxycarbonyl)-4-{[1-(t-butoxycarbonyl)-6-fluoro-2-indolyl]-methylene}-piperidine (Compound 69a)

The title compound was prepared following the method reported above for Compound 68a using 1-(t-butoxycarbonyl)-6-fluoro-2-indolylboronic acid. Yield: 98%.

### 4-[(6-fluoro-2-indolyl)-methylene]-piperidine (Compound 69b)

The title product was synthesised as reported for Compound 47b starting from Compound 69a instead of Compound 47a and heating at 65° C. for 10 hours. Purified by RP chromatography (SP1®-Biotage) eluting with CH₃CN—H₂O 65:35 and used in the next step without further characterization. Yield: 36%.

### 1-(6-methyl-3-nitro-2-pyridyl)-4-[(6-fluoro-2-indolyl)-methylene]-piperidine

The title product was synthesised as reported for the Compound of Example 47 starting from Compound 69b instead of Compound 47b. After the work-up, the crude was purified by automated flash chromatography (SP1®-Biotage) eluting with a PE-EtOAc 9:1, affording the title compound as orange solid. Yield: 33%.

¹H-NMR (CDCl₃, δ): 2.50 (s, 3H), 2.60 (t, J=5.75 Hz, 2H), 2.88 (t, J=5.62 Hz, 2H), 3.57 (dt, J=16.20, 5.96 Hz, 4H), 6.28 (s, 1H) 6.44 (s, 1H) 6.61 (d, J=8.07 Hz, 1H) 6.84-6.91 (m, 1H), 7.03 (dd, J=9.41, 1.83 Hz, 1H), 7.49 (dd, J=8.80, 5.38 Hz, 1H) 7.93 (bs, 1H), 8.10 (d, J=8.07 Hz, 1H).

MS: [M+H]⁺=367.39

### Example 70

### 1-(6-methyl-3-nitro-2-pyridyl)-4-{[3-(3-chlorophenyl)-1,2,4-oxadiazol-5-yl]-methylene}-piperidine

### 1-(t-butoxycarbonyl)-4-{2-[(amino)(3-chlorophenyl)-methylene-aminooxy]-2-oxo-ethylidene}-piperidine (Compound 70a)

A solution of 1-(t-butoxycarbonyl)-4-(2-chloro-2-oxo-ethylidene)-piperidine (371 mg; 1.43 mmol) and 3-chloro-N′-hydroxybenzamidine (195 mg, 1.14 mmol) in anhydrous CHCl₃ (8 ml) containing 3 Å molecular sieves was stirred at r.t. for 3 hours, poured into water, extracted with CHCl₃, dried (Na₂SO₄) and evaporated to dryness in vacuo. The crude was purified by automated flash chromatography (Horizon®-Biotage) eluting with a PE-EtOAc gradient from 7:3 to 4:6, affording 140 mg of the title compound.

MS: [M+H]⁺=394.07

### 1-(t-butoxycarbonyl)-4-{[3-(3-chlorophenyl)-1,2,4-oxadiazol-5-yl]-methylene}-piperidine (Compound 70b)

A mixture of Compound 70a (140 mg, 0.36 mmol), tetra-butylammonium fluoride (120 mg, 0.46 mmol), 3 Å molecular sieves (100 mg) in anhydrous THF (5 ml) was stirred at r.t. for 2 hours, quenched with an aqueous solution of NH₄Cl, extracted with EtOAc, dried (Na₂SO₄) and evaporated to dryness in vacuo. The crude was purified by automated flash chromatography (Horizon®-Biotage), eluting with a PE-EtOAc gradient from 95:5 to 80:20 and affording 140 mg of the title compound.

MS: [M+H]⁺=376.10

### 4-{[3-(3-chlorophenyl)-1,2,4-oxadiazol-5-yl]-methylene}-piperidine (Compound 70c)

The title product was synthesized following the same procedure reported above for the Compound 47b, but using Compound 70b instead of Compound 47a. The crude was used for the next step without further purification. Yield: 89%.

MS: [M+H]⁺=276.01

### 1-(6-methyl-3-nitro-2-pyridyl)-4-{[3-(3-chlorophenyl)-1,2,4-oxadiazol-5-yl]-methylene}-piperidine

The title product was synthesized following the same procedure reported above for the compound of Example 47, but using Compound 70c instead of Compound 47b. The crude was purified by automated flash chromatography (Horizon®-Biotage) eluting with a PE-EtOAc gradient from 95:5 to 70:30 and affording 35 mg of the title compound.

¹H-NMR (CDCl₃, δ): 2.53 (s, 3H) 2.69 (t, J=5.50 Hz, 2H) 3.33 (t, J=5.62 Hz, 2H) 3.65 (q, J=6.11 Hz, 4H) 6.42 (s, 1H) 6.67 (d, J=8.07 Hz, 1H) 7.45 (t, J=7.80 Hz, 1H) 7.50 (d, J=8.30 Hz, 1H) 8.02 (d, J=7.58 Hz, 1H) 8.11-8.18 (m, 2H)

MS: [M+H]⁺=412.13

### Example 71

### 1-(6-methyl-3-nitro-2-pyridyl)-4-{[5-(4-pyridyl)-1,2,4-oxadiazol-3-yl]-methylene}-piperidine

### Diethyl [5-(4-pyridyl)-1,2,4-oxadiazol-3-yl]-methylphosphonate (Compound 71a)

To a solution of Compound 44a (125 mg, 0.596 mmol) and DIPEA (208 μl, 1.19 mmol) in 3 ml of CH₃CN was added portionwise isonicotinoyl chloride.HCl (117 mg, 0.657 mmol) and the mixture was stirred at r.t. overnight, until the complete consumption of Compound 44a and the formation of the O-acyl derivative. Afterwards, tetra-n-butylammonium fluoride hydrate (0.51 g, 1.79 mmol) and 2 g of molecular sieves 4 Å were added and the mixture was stirred at r.t. for 3 h. The molecular sieves were filtered off and the solution was evaporated in vacuo to dryness to afford a residue, which was purified by automated flash chromatography (Horizon®-Biotage) eluting with CHCl₃-1.4M NH₃ sol. in MeOH 100:1 affording 116 mg of the title product as yellow oil.

MS: [M+H]⁺=298.21

### 1-(6-methyl-3-nitro-2-pyridyl)-4-{[5-(4-pyridyl)-1,2,4-oxadiazol-3-yl]-methylene}-piperidine

The title compound was prepared following the procedure reported for the Compound of Example 1, but using Compound 71a instead of Compound 1b. After the usual work-up procedure, the crude was purified by automated flash chromatography (Horizon®-Biotage) eluting with PE-EtOAc 65:35, affording the title compound as yellow needles (76.5%).

¹H-NMR (CDCl₃, δ): 2.50 (s, 3H), 2.65 (t, J=5.62 Hz, 2H), 3.22 (t, J=5.75 Hz, 2H), 3.55-3.68 (m, 4H), 6.37 (s, 1H), 6.64 (d, J=8.07 Hz, 1H), 8.07 (d, J=4.89 Hz, 2H), 8.12 (d, J=8.07 Hz, 1H), 8.91 (br. s., 2H).

MS: [M+H]⁺=379.14

### Example 72

### 1-(6-methyl-3-nitro-2-pyridyl)-4-{[5-(2-pyridyl)-1,2,4-oxadiazol-3-yl]-methylene}-piperidine

### Diethyl [5-(2-pyridyl)-1,2,4-oxadiazol-3-yl]-methylphosphonate (Compound 72a)

The title compound was prepared following the procedure reported for Compound 71a, but using pyridine-2-carbonyl chloride.HCl instead of isonicotinoyl chloride.HCl. After the usual work-up procedure, the crude was purified by automated flash chromatography (Horizon®-Biotage) eluting with CHCl₃-1.4M NH₃ sol. in MeOH 100:0.8, affording the title compound as a yellow oil (35.6%).

MS: [M+H]⁺=298.10

### 1-(6-methyl-3-nitro-2-pyridyl)-4-{[5-(2-pyridyl)-1,2,4-oxadiazol-3-yl]-methylene}-piperidine

The title compound was prepared following the procedure reported for the Compound of Example 1, but using Compound 72a instead of Compound 1b. After the usual work-up procedure, the crude was purified by automated flash chromatography (Horizon®-Biotage) eluting with PE-EtOAc 7:3, affording the title compound as yellow needles (94%).

¹H-NMR (CDCl₃, δ): 2.50 (s, 3H), 2.64 (t, J=5.62 Hz, 2H), 3.23 (t, J=5.62 Hz, 2H), 3.53-3.70 (m, 4H), 6.39 (s, 1H), 6.62 (d, J=8.07 Hz, 1H), 7.48-7.58 (m, 1H), 7.88-7.98 (m, 1H), 8.11 (d, J=8.31 Hz, 1H), 8.26 (d, J=7.82 Hz, 1H), 8.87 (d, J=4.65 Hz, 1H).

MS: [M+H]⁺=379.14

### Example 73

### 1-(6-methyl-3-nitro-2-pyridyl)-4-{[5-(3-pyridyl)-1,2,4-oxadiazol-3-yl]-methylene}-piperidine

### Diethyl (5-pyridin-3-yl-1,2,4-oxadiazol-3-yl)methylphosphonate (Compound 73a)

The title compound was prepared following the procedure reported for the Compound 71a, but using nicotinoyl chloride.HCl instead of isonicotinoyl chloride.HCl. After the usual work-up procedure, the crude was purified by automated flash chromatography (Horizon®-Biotage) eluting with CHCl₃-1.4M NH₃ sol. in MeOH 100:1, affording the title compound as a yellow oil (34%).

MS: [M+H]⁺=298.1

### 1-(6-methyl-3-nitro-2-pyridyl)-4-{[5-(3-pyridyl)-1,2,4-oxadiazol-3-yl]-methylene}-piperidine

The title compound was prepared following the procedure reported for the Compound of Example 1, but using Compound 73a instead of Compound 1b. After the usual work-up procedure, the crude was purified by automated flash chromatography (Horizon®-Biotage) eluting with PE-EtOAc 6:4, affording the title compound as yellow needles (85%).

¹H-NMR (CDCl₃, δ): 2.50 (s, 3H), 2.65 (t, J=5.50 Hz, 2H), 3.23 (t, J=5.75 Hz, 2H), 3.62 (q, J=5.14 Hz, 4H), 6.37 (s, 1H), 6.63 (d, J=8.07 Hz, 1H), 7.53 (dd, J=7.82, 4.89 Hz, 1H), 8.12 (d, J=8.07 Hz, 1H), 8.45 (d, J=8.07 Hz, 1H), 8.86 (d, J=4.65 Hz, 1H) 9.41 (s, 1H).

MS: [M+H]⁺=379.00

### Example 74

### 1-(6-methyl-3-nitro-2-pyridyl)-4-{1-[5-(3-chlorophenyl)-1,2,4-oxadiazol-3-yl]-propylidene}-piperidine

### 1-(t-butoxycarbonyl)-4-{1-[(amino)(hydroxyimino)methyl]-propylidene}-piperidine (Compound 74a)

To a colorless solution of 1-(t-butoxycarbonyl)-4-(1-cyanopropylidene)-piperidine (1.03 g, 4.12 mmol) in 20 ml of EtOH were added 36 ml of DIPEA and portionwise, hydroxylamine HCl (14.3 g, 206 mmol). The reaction mixture was stirred at reflux for 5 h. Afterwards, the solvent was removed. The residue was taken up with water, extracted with EtOAc (5 times), washed with brine and dried over sodium sulphate. After evaporation to dryness, the crude was purified by automated flash chromatography (Isolera®-Biotage) eluting with a PE-EtOAc gradient from 4:6 to 2:8 affording 280 mg (24%) of the title compound as E/Z mixture as white-greenish solid.

MS: [M+H]⁺=284.15

### 1-(t-butoxycarbonyl)-4-{1-[(amino)(3-chlorobenzoyloxy-imino)methyl]-propylidene}-piperidine (Compound 74b)

The title compound was synthesized following the same procedure reported for Compound 44b, but using Compound 74a instead of Compound 44a. After the usual work-up, the crude was purified by automated flash chromatography (Isolera®-Biotage) eluting with PE-EtOAc 65:35 affording the title compound as a yellowish solid. Yield: 23.6%.

MS: [M+H]⁺=422.12

### 1-(t-butoxycarbonyl)-4-{1-[5-(3-chlorophenyl)-1,2,4-oxadiazol-3-yl]-propylidene}-piperidine (Compound 74c)

The title compound was synthesized following the same procedure reported for Compound 44c, but using Compound 74b using TBAF (1M sol. in anhydrous THF) and conducting the reaction at r.t. stirring for 3 h. The orange solid obtained from evaporation to dryness of the reaction mixture was purified by automated flash chromatography (Isolera®-Biotage) eluting with a PE-EtOAc from 98:2 to 95:5 affording the title compound as a colorless oil. Yield: 40%.

MS: [M+H]⁺=404.13

### 4-{1-[5-(3-chlorophenol)-1,2,4-oxadiazol-3-yl]-propylidene}-piperidine (Compound 74d)

The title product was synthesized following the same procedure reported above for the Compound 47b, but using Compound 74c instead of Compound 47a and stirring at r.t. for 4 h. The crude was used for the next step without further purification. Yield: 100%.

MS: [M+H]⁺=304.15

### 1-(6-methyl-3-nitro-2-pyridyl)-4-{1-[5-(3-chlorophenyl)-1,2,4-oxadiazol-3-yl]-propylidene}-piperidine

The title product was synthesized following the same procedure reported above for the compound of Example 47, but using Compound 74d instead of Compound 47b. The crude was purified by automated flash chromatography (Horizon®-Biotage) eluting with a PE-EtOAc 95:5 affording the title compound. Yield: 82%.

¹H-NMR (CDCl₃, δ): 1.10 (t, J=7.34 Hz, 3H), 2.49 (s, 3H), 2.63 (q, J=7.34 Hz, 2H), 2.72 (t, J=5.26 Hz, 2H), 2.88 (t, J=5.14 Hz, 2H), 3.52 (t, J=5.38 Hz, 2H), 3.62 (t, J=5.38 Hz, 2H), 6.59 (d, J=8.07 Hz, 1H), 7.50 (t, J=7.83 Hz, 1H), 7.59 (d, J=8.07 Hz, 1H), 8.09 (d, J=8.31 Hz, 1H), 8.06 (d, J=8.56 Hz, 1H), 8.18 (s, 1H).

MS: [M+H]⁺=440.17

### Example 75

### 1-(6-methyl-3-nitro-2-pyridyl)-4-{1-[3-(3-chlorophenyl)-1,2,4-oxadiazol-3-yl]-ethylidene}-piperidine

### Diethyl 1-[5-(3-chlorophenol)-1,2,4-oxadiazol-3-yl]-ethylphosphonate (Compound 75a)

The title product was prepared following the synthetic method described for Compound 39a, but using Compound 44c instead of Compound 18a. The resultant orange oil was used in the next step without further purification.

MS: [M+H]⁺=345.01

### 1-(6-methyl-3-nitro-2-pyridyl)-4-{1-[5-(3-chlorophenyl)-1,2,4-oxadiazol-3-yl]-ethylidene}-piperidine

The title product was synthesised following the same procedure reported above for the compound of Example 1 but using Compound 75a instead of Compound 1b. Similar work-up and purification by automated flash chromatography (Isolera®-Biotage) eluting with a PE-EtOAc 95:5 affording the title compound as yellow solid. Yield: 19%.

¹H-NMR (CDCl₃, δ): 2.18 (s, 3H), 2.49 (s, 3H), 2.73 (t, J=5.87 Hz, 2H), 3.01 (t, J=5.62 Hz, 2H), 3.51 (t, J=5.87 Hz, 2H), 3.64 (t, J=5.99 Hz, 2H), 6.59 (d, J=8.31 Hz, 1H), 7.47-7.54 (m, 1 H), 7.56-7.63 (m, 1H), 8.06 (d, J=7.82 Hz, 1H), 8.10 (d, J=8.31 Hz, 1H), 8.17 (s, 1H).

MS: [M+H]⁺=426.15

### Example 76

### 1-(3-cyano-6-methyl-2-pyridyl)-4-{[3-(3-chlorophenyl)-1,2,4-oxadiazol-5-yl]-methylene}-piperidine

### 1-(3-cyano-6-methyl-2-pyridyl)-4-oxo-piperidine (Compound 76a)

To a mixture of K₂CO₃ (774 mg, 5.6 mmol), 2-chloro-6-methynicotinonitrile (305 mg, 2 mmol) and piperidin-4-one.HCl.H₂O (376 mg, 4 mmol) stirred under a nitrogen atmosphere, was added via syringe the pre-activated catalyst obtained from Pd(OAc)₂ (4.6 mg, 0.02 mmol) and X-Phos (29.5 mg, 0.06 mmol) by adding 4 ml di t-BuOH and 1.5μΛ of degassed water and heating at 110° C. for 1.5 min. The resultant orange suspension was stirred at 110° C. for 2 h. Another 2% of preformed catalyst was added and refluxing kept on for 5 h. The suspension became yellow. After having added additional 0.8 eq. (251 mg.) of piperidinone.HCl.H₂O and 1.6 mmol (221 mg) of K₂CO₃ and refluxed for 1 h, the reaction mixture was cooled, diluted with EtOAc, washed with water, dried, evaporated to dryness in vacuo and purified by automated flash chromatography (Isolera®-Biotage) eluting with a gradient PE-EtOAc from 85:15 to 80:20 affording the title compound as yellowish solid (34 mg). Yield: 7.9%.

MS: [M+H]⁺=216.15

### 1-(3-cyano-6-methyl-2-pyridyl)-4-{[3-(3-chlorophenyl)-1,2,4-oxadiazol-5-yl]-methylene}-piperidine

The title product was synthesised following the same procedure reported above for the compound of Example 1 but using Compound 75a instead of Compound 1b. Similar work-up and purification by automated flash chromatography (Isolera®-Biotage) eluting with a PE-EtOAc 9:1 afforded the title compound as white-beige solid. Yield: 75.3%.

¹H-NMR (CDCl₃, δ): 2.48 (s, 3H), 2.63 (t, J=5.62 Hz, 2H), 3.24 (t, J=5.62 Hz, 2H), 3.85-3.97 (m, 4H), 6.34 (s, 1H), 6.63 (d, J=7.82 Hz, 1H), 7.51 (t, J=7.60 Hz, 1H), 7.59 (d, J=8.10 Hz, 1H), 7.68 (d, J=7.82 Hz, 1H), 8.07 (d, J=7.82 Hz, 1H) 8.17 (s, 1H),

MS: [M+H]⁺=392.06

### Example 77

### Affinity of Selected Antagonists for mGlu5 Receptor Subtype

Radioligand Binding Assay at metabotropic glutamate receptor 5 in rat brain.

### Methods

a) Membrane preparation: male Sprague Dawley rats (200-300 g, Charles River, Italy) were killed by cervical dislocation and the forebrain (cortex, striatum and hippocampus) was homogenized (2×20 sec) in 50 vols of cold 50 mM Tris buffer pH 7.4, using a Politron homogenizer (Kinematica). Homogenates were centrifuged at 48000×g for 15 min, resuspended in 50 vols of the same buffer, incubated at 37° C. for 15 min and centrifuged and resuspended two more times. The final pellets were frozen and stored at −80° C. until use.

b) Binding assay: pellets from rat forebrain were resuspended in 100 vols of 20 mM HEPES, 2 mM MgCl₂, 2 mM CaCl₂, pH 7.4. The membranes were incubated in a final volume of 1 ml for 60 min at 25° C. with 4 nM [³H]MPEP in the absence or presence of competing drugs. Non-specific binding was determined in the presence of 10 μM MPEP (Spooren W. et al. Trends Pharmacol Sci. 22, 331-337, 2001). The incubation was stopped by the addition of cold Tris buffer pH 7.4 and rapid filtration through 0.5% polyethyleneimine pretreated Filtermat 1204-401 (Wallac) filters. The filters were then washed with cold buffer and the radioactivity retained on the filters was counted by liquid scintillation spectrometry.

c) Data Analysis: the inhibition of specific binding of the radioligands by the tested compounds was analyzed to estimate the inhibitory concentration 50% (IC₅₀) value by using the non-linear curve-fitting software Prism 4.0 (Graphpad, San Diego, Calif.). The IC₅₀ value was converted to an affinity constant (Ki) by the equation of Cheng & Prusoff (Cheng, Y. C.& Prusoff, W. H. Biochem. Pharmacol. 22, 3099-3108, 1973).

### Results

The affinity (Ki) of the compounds of the instant invention in particular the compounds of Examples 1 to 76, for mGlu5 receptor is between 0.1 and 1000 nM. For instance, Compound of Example 1 has a Ki of 0.37 nM.

### Example 78

### Affinity of Selected Antagonists for mGlu1 Receptor Subtype

Radioligand Binding Assay at metabotropic glutamate receptor 1 in rat brain.

### Methods

a) Membrane preparation: male Sprague Dawley rats (200-300 g, Charles River, Italy) were killed by cervical dislocation and the cerebella were homogenized (2×20 sec) in 50 vols of cold 50 mM Tris buffer pH 7.4, using a Politron homogenizer (Kinematica). Homogenates were centrifuged at 48000×g for 15 mM, resuspended in 50 vols of the same buffer, incubated at 37° C. for 15 min and centrifuged and resuspended two more times. The final pellets were frozen and stored at −80° C. until use.

b) Binding assay: pellets from rat cerebellum were resuspended in 50 mM Tris, 1.2 mM MgCl₂, 2 mM CaCl₂, pH 7.4; membranes were incubated in a final volume of 1 ml for 30 min at 0° C. with 1.5 nM [³H] R214127 in absence or presence of competing drugs. Non-specific binding was determined in the presence of 1 μM 8214127 (Lavreysen H et al Mol. Pharmacol. 63:1082-1093, 2003). The incubation was stopped by the addition of cold Tris buffer pH 7.4 and rapid filtration through 0.5% polyethyleneimine pretreated Filtermat 1204-401 (Wallac) filters. The filters were then washed with cold buffer and the radioactivity retained on the filters was counted by liquid scintillation spectrometry.

c) Data Analysis: the inhibition of specific binding of the radioligands by the tested compounds was analyzed to estimate the inhibitory concentration 50% (IC₅₀) value by using the non-linear curve-fitting software Prism 4.0 (Graphpad, San Diego, Calif.). The IC₅₀ value was converted to an affinity constant (Ki) by the equation of Cheng & Prusoff (Cheng, Y. C.& Prusoff, W. H. Biochem. Pharmacol. 22, 3099-3108, 1973).

### Results

The affinity of the compounds, in particular the compounds of Examples 1 to 76, of the instant invention for mGlu1 receptor is at least 10 times lower than their affinity for mGlu5 receptor.

### Example 79

### Affinity of Selected Antagonists for Group II (mGlu2+ mGlu3) Receptor Subtypes

Radioligand Binding Assay at Group II metabotropic glutamate receptors in rat brain.

### Methods

a) Membrane preparation: male Sprague Dawley rats (200-300 g, Charles River, Italy) were killed by cervical dislocation and the forebrain (cortex, striatum and hippocampus) was homogenized (2×20 sec) in 50 vols of cold 50 mM Tris buffer pH 7.4, using a Politron homogenizer (Kinematica). Homogenates were centrifuged at 48000×g for 15 mM, resuspended in 50 vols of the same buffer, incubated at 37° C. for 15 mM and centrifuged and resuspended two more times. The final pellets were frozen and stored at −80° C. until use.

b) Binding assay: pellets of rat forebrain were washed three times with ice-cold assay buffer (10 mM potassium phosphate+100 nM potassium bromide, ph 7.6). Final pellets were resuspended in 200 vols of the assay buffer and membranes incubated in a final volume of 1 ml for 30 min at 0° C. with 1 nM [³H]LY341495 in the absence or presence of competing drugs. Non-specific binding was determined in the presence of 1 mM 1-glutamate (Wright R. A. et al. J. Pharmacol. Exp. Ther. 298:453-460, 2001; Mutel V et al. J. Neurochem. 75, 2590-2601, 2000). The incubation was stopped by the addition of cold Tris buffer pH 7.4 and rapid filtration through 0.5% polyethyleneimine pretreated Filtermat 1204-401 (Wallac) filters. The filters were then washed with cold buffer and the radioactivity retained on the filters was counted by liquid scintillation spectrometry.

c) Data Analysis: the inhibition of specific binding of the radioligands by the tested compounds was analyzed to estimate the inhibitory concentration 50% (IC₅₀) value by using the non-linear curve-fitting software Prism 4.0 (Graphpad, San Diego, Calif.). The IC₅₀ value was converted to an affinity constant (Ki) by the equation of Cheng & Prusoff (Cheng, Y. C.& Prusoff, W. H. Biochem. Pharmacol. 22, 3099-3108, 1973).

### Results

The compounds of the instant invention, in particular the compounds of Examples 1 to 76, did not affect [³H]LY341495 binding to Group II (mGlu2+ mGlu3) metabotropic glutamate receptors up to 1000 nM.

### Example 80

### Determination of Functional Activity at mGlu5 Receptor as Accumulation of Inositol Phosphate

To determine the mode of action (agonist, antagonist or inverse agonist) of the test compounds at mGlu5 receptor, the concentration dependence of the stimulation of inositol phosphate production in response to the agonist (glutamate or quisqualic acid) was compared in the absence and presence of different concentrations of the test compounds themselves, measured in cells expressing mGlu5 receptor.

The cells were preincubated with the glutamate-degrading enzyme (1 U/ml glutamate pyruvate transaminase) and 2 mM pyruvate to avoid the possible action of glutamate released from the cells. The stimulation was then conducted in a medium containing 10 mM LiCl, and different concentrations of the agonist (glutamate or quisqualic acid) or compounds to be tested for agonistic activity.

When antagonist activity was studied, test compounds were added to cell cultures 20 mM prior to the addition of the agonist and further incubated in the presence of the agonist.

The incubation was stopped adding ice cold perchloric acid then samples were neutralized, centrifuged and the supernatant utilized for the determination of inositol phosphate (IP) accumulation using the The Biotrak D-myo-Inositol 1,4,5-trisphosphate assay system from Amersham Biosciences. D-myo-Inositol 1,4,5-trisphosphate (IP₃) may be measured in the range 0.19-25 pmol (0.08-10.5 ng) per tube. In the assay, unlabelled IP₃ competes with a fixed amount of [³H]-labelled IP₃ for a limited number of bovine adrenal IP₃ binding proteins. The bound IP₃ is then separated from the free IP₃ by centrifugation, which brings the binding protein to the bottom of the tube. The free IP₃ in the supernatant can then be discarded by simple decantation, leaving the bound fraction adhering to the tube. Measurement of the radioactivity in the tube enables the amount of unlabelled IP₃ in the sample to be determined by interpolation from a standard curve.

EC₅₀/IC₅₀ were determined by nonlinear regression analysis using the software Prism 4.0 (Graphpad, San Diego, Calif.).

### Results

The compounds of the instant invention, in particular the compounds of Examples 1 to 76, showed antagonistic activity toward mGlu5.

### Example 81

### Effect on Cystometry in Conscious Rats

### Methods:

Male Sprague-Dawley rats [Crl: CD® (SD) IGS BR] of 300-400 g b.w. supplied by Charles River Italia were used. The animals were housed with free access to food and water and maintained on a forced 12-hour-light/12-hour-dark cycle at 22-24° C. of temperature, except during the experiment. To quantify urodynamic parameters in conscious rats, cystometrographic studies were performed according to the procedure previously reported (Guarneri et al., Pharmacol. Res. 24: 175, 1991).

Briefly, the rats were anaesthetised by intraperitoneal administration of 3 ml/kg of Equithensin solution (pentobarbital 30 mg/kg and chloral hydrate 125 mg/kg) and placed in a supine position. An approximately 10 mm long midline incision was made in the shaved and cleaned abdominal wall. The urinary bladder was gently freed from adhering tissues, emptied and then cannulated via an incision in the bladder body, using a polyethylene cannula (0.58 mm internal diameter, 0.96 mm external diameter) which was permanently sutured with silk thread. The cannula was exteriorised through a subcutaneous tunnel in the retroscapular area, where it was connected to a plastic adapter in order to avoid the risk of removal by the animal. For drug testing, the rats were utilised one day after implantation.

On the day of the experiment, the rats were placed in modified Bollman cages, i.e., restraining cages that were large enough to permit the rats to adopt a normal crouched posture, but narrow enough to prevent turning around. After a stabilisation period of about 20 minutes, the free tip of the bladder cannula was connected through a T-shaped tube to a pressure transducer (Statham P23XL) and to a peristaltic pump (Gilson Minipuls 2) for continuous infusion of a warm (37° C.) saline solution into the urinary bladder, at a constant rate of 0.1 ml/minute. The intraluminal-pressure signal during infusion of saline into the bladder (cystometrogram) was continuously recorded on a polygraph (Rectigraph-8K San-ei with BM614/2 amplifier from Biomedica Mangoni) or stored on PC by data acquisition system (PowerLab, Chart 4 software, AD Instruments). From the cystometrogram, bladder volume capacity (BVC) was evaluated. BVC (in ml) is defined as the volume of saline infused into the bladder necessary to induce detrusor contraction followed by micturition. Basal BVC value was evaluated as the mean of the values observed in the cystometrograms recorded in an initial period of 30-60 minutes. At this point in the assay, the infusion was interrupted and the test compounds were administered orally by a stomach tube. The bladder infusion restarted and changes in BVC were evaluated from the mean values obtained in the cystometrograms observed during 1, 2, and 3 hours after treatment.

The compounds were administered in a volume of 2 ml/kg. Groups of control animals received the same amount of vehicle corresponding to a solution 0.5% methocel in water.

Under the given test conditions, measurement of BVC is equivalent to measurement of interval time between micturitions.

### Statistical Analysis

Each experimental group was composed of 4-11 animals. All data were expressed as mean±standard error. The percent change of BVC versus the basal value, as well as Δ value (difference in ml) of BVC (BVC at time “x” minus basal value), were also evaluated for each rat/time. Statistical analysis on BVC values, as well as on Δ values, was performed by S.A.S./STAT software, version 6.12. The difference between vehicle and active treatment effect was evaluated on Δ values of BVC, whereas the difference between the values at different times versus the basal values was evaluated on original BVC data.

### Results

The compound of Example 29 proved effective in increasing the bladder volume capacity.

### Example 82

### Plasma Extravasation in the Dura Mater of Rats Induced by Electrical Stimulation of the Trigeminal Ganglion

Electrical stimulation of the trigeminal ganglion induces inflammation in the dura mater which causes plasma extravasation. This animal model is widely accepted for testing drugs useful in migraine.

Male Wistar rats weighing 175-190 g are anaesthetised with 50 mg/kg i.p. of pentobarbital and the jugular vein is cannulated for injection of drugs. The animals are placed in a stereotaxic frame. Symmetrical boreholes are drilled 3.0 mm laterally and 3.2 mm posteriorly from bregma and the electrodes are lowered 9.5 mm from dura mater. The test compound or control-vehicle solution are administered intravenously 10 min prior to electrical stimulation of the right trigeminal ganglion (5 min; 2.0 mA, 5 Hz, 5 ms duration and Evans blue (30 mg/kg i.v.), is given 5 min prior to electrical stimulation as a marker of plasma protein extravasation. 15 minutes after the end of the stimulation period the animals are perfused with 50 ml saline via the left cardiac ventricle to remove intravascular Evans blue. The dura mater is removed, blotted dry and weighed. Tissue Evans blue is extracted in 0.3 ml formamide at 50° C. for 24 h. Dye concentrations are measured with a spectrophotometer at 620 nm wavelength, interpolated on a standard curve and expressed as ng Evans blue content per mg tissue weight.

Extravasation is expressed as the quotient calculated by dividing the Evan's blue content of the stimulated side by the Evan's blue content of the unstimulated side.

### Example 83

### GERD Model in Dogs

Beagle dogs are equipped with a chronic esophagostomy to allow passage of a manometric catheter and a pH probe along the esophagus and the stomach.

Following recording of the basal pressure of the Lower Esophageal Sphincter and the stomach, compounds under evaluation and vehicle for control are administered by intravenous route. Transient Lower Esophageal Sphincter Relaxations (TLESRs) and acid reflux are induced by infusion of an acidified meal followed by stomach distension using a peristaltic pump infusing air at 40 ml/min, in accordance to Stakeberg J. and Lehmann A., (Neurogastroenterol. Mot. (1999) 11: 125-132). Active compounds reduce dose-dependently the frequency of TLESRs and TLESRs associated with acid reflux. The activity is determined as % inhibition of both parameters as compared to vehicle control.

### Example 84

### Vogel Conflict Test in Rat

The method, which detects anxiolytic activity, follows that described by Vogel et al. as “Anxiolytics increase punished drinking” (Vogel J. R., Beer B., Clody D. E. A simple and reliable conflict procedure for testing anti-anxiety agents Psychopharmacologia, 21, 1-7, 1971). Rats were deprived of water for approximately 48 hours and were then placed individually into a transparent Plexiglas enclosure (15×32×34 cm) with a floor consisting of stainless steel bars (0.4 cm) spaced 1 cm apart. The back wall of the enclosure was made of opaque Plexiglas thereby concealing the observer from the experimental animal. In the centre of the opposite wall, 5 cm above the floor, a metal water spout protruded into the cage and was connected to one pole of a shock generator. The other pole of the shock generator was connected to the metal grid floor. The rat was left to explore until it founded the water spout. Then, every time it drank, it received a slight electric shock (1.7 mA, 1 s) 2 seconds after it started lapping. The number of punished drinks was counted during a 3 minute test.

The test was performed blind. The test compounds were administered p.o. 60 minutes before the test, and compared with a vehicle control group.

### Example 85

### Operant Alcohol Self-Administration Training

The method was used to assess a substance abuse model and to detect the activity of the compounds of the invention in preventing this behavior.

Rats were trained to orally self-administer ethanol by using a modification of a training protocol described previously by Samson (1986). Briefly, rats were deprived of water for 12 h prior to training sessions for three consecutive days and were trained to respond for a 0.1-ml drop of 0.2% (w/v) saccharin solution on both levers under a fixed ratio 1 (FR1) schedule of reinforcement. After this initial training, water deprivation was terminated, and animals had free access to food and water in their home cages throughout the subsequent training and testing. Non-deprived rats were given two additional saccharin sessions to confirm that they had acquired responding for saccharin before ethanol self administration training started. Then, during the next three sessions, responses at the right lever resulted in the delivery of 0.1 ml of 5% (w/v) ethanol+0.2% saccharin solution. Responses at the left lever were recorded but had no programmed consequences. Thereafter, the concentration of ethanol was increased first to 8% and then to 10% w/v and the concentration of saccharin was decreased until saccharin was eliminated completely from the drinking solution.

The final schedule of reinforcement for the 10% w/v ethanol concentration was similar to the training schedule except that a stimulus light was added. Thus, during the 30-min sessions responses on the active lever resulted in the delivery of 0.1 ml of ethanol and, in addition, in the illumination of the stimulus light for 3 s. The left lever remained inactive. When rats had reached stable ethanol self-administration under these conditions, the effects of the tested compounds after i.p. administration on ethanol self administration were examined. The agonists were administered 30 min before start of the self-administration session.

### Example 86

### Neuropathic Pain Test (Bennett) in the Rat

The method, which detects analgesic activity in rats with neuropathic pain, follows that described by Bennett and Xie (Bennett G. J., Xie Y. K., A peripheral mononeuropathy in rat that produces disorders of pain sensation like those seen in man, Pain, 33, 87-107, 1988).

Chronic constriction injury of the common sciatic nerve in rats is associated with hyperalgesia, allodynia and spontaneous pain, and constitutes therefore a model for peripheral neuropathic pain in humans. Antihyperalgesics reduce these chronic signs of pain hypersensitivity. Rats (150-200 g) were anesthetized (sodium pentobarbital 40 mg/kg i.p.) and an incision at mid-thigh level was performed to expose the common left sciatic nerve. Four ligatures spaced 1 mm apart were loosely tied around the sciatic nerve. The wound was then sutured. The rats were allowed to recover. One week after the surgery, when the chronic pain state was fully installed, rats were submitted consecutively to tactile and thermal stimulation of both hindpaws.

For tactile stimulation, the animal was placed under an inverted acrylic plastic box (17×11×14 cm) on a grid floor. The tip of an electronic Von Frey probe was then applied with increasing force to the non-inflamed and inflamed hindpaws and the force inducing paw-withdrawal was automatically recorded. This procedure was carried out 3 times and the mean force per paw was calculated.

For thermal stimulation, the apparatus consists of individual acrylic plastic boxes (17×11×14 cm) placed upon an elevated glass floor. A rat was placed in the box and left free to habituate for 10 minutes. A mobile infrared radiant source (96±10 mW/cm²) was then focused first under the non-lesioned and then the lesioned hindpaw and the paw-withdrawal latency was automatically recorded. In order to prevent tissue damage the heat source was automatically turned off after 45 seconds.

Prior to receiving drug treatment all animals were submitted to tactile stimulation of the hindpaws and assigned to treatment groups matched on the basis of the pain response of the lesioned hindpaw.

8 rats were studied per group. The test was performed blind.

Test compounds were administered p.o. 60 minutes before the test, and compared with a vehicle control group (0.5% carboxymethylcellulose (CMC) in distilled water).

### Example 87

### Limbic Epileptogenesis in a Mouse Model

FMRP (fragile X mental retardation protein) plays a critical role in suppressing limbic epileptogenesis and predict that the enhanced susceptibility of patients with FXS to epilepsy is a direct consequence of the loss of an important homeostatic factor that mitigates vulnerability to excessive neuronal excitation.

Kindling experiments were conducted in FMR1 mutant mice (knockout mice with reduced expression of mGluR5) during the light phase of the cycle on 12-week-old adult mice. A twisted bipolar electrode was implanted into the right amygdale (coordinates: 2.9 mm lateral and 1.2 mm posterior to bregma, 4.6 mm below dura) of animals under pentobarbital (60 mg/kg) anesthesia. Animals were then given 10 days to recover. The electrographic seizure threshold (EST) for each individual mouse was determined by applying 1-s train of 1-ms biphasic rectangular pulses at 60 Hz beginning at 50 1 A. Additional stimulations increasing by 101 A were administered at 2-min intervals until an electrographic seizure lasting at least 5 s was evoked. Stimulations at the EST intensity were subsequently applied once daily. EEGs and behavioral seizures were observed and recorded. The severity of the behavioral manifestations of seizures was classified according to the criteria of Racine (1972). Fully kindled is defined by the occurrence of 3 consecutive seizures of class 4 or greater. All surgery and kindling procedures were performed blind to genotype.

Unstimulated control animals of each genotype underwent surgical implantation of an electrode in the amygdala and were handled identically but were not stimulated. Electrode placement was confirmed by methyl green pyronine-Y staining. Data derived from animals with correct electrode placement were analyzed.

Tested drugs were administered by intraperitoneal injection 30 min before a class 5 seizure-inducing stimulation.

### Example 88

### 6-Hydroxydopamine-Lesioned Rats

This model is a reliable and robust model for reproducing Parkinson's lesions in the brain and for studying the protecting effects of drugs.

Female Sprague-Dawley rats (180-220 g) underwent stereotaxic surgery to produce lesions of the nigrostriatal system. Within each experimental group, half of the animals received an intrastriatal injection of 6-hydroxydopamine (6-OHDA) with 0.01% ascorbic acid in saline (lesioned animals) while the remaining animals received an intrastriatal injection of 0.01% ascorbic acid in saline (control animals). Surgical procedure for unilateral intrastriatal injection of 6-OHDA: rats were anaesthetized with sodium pentobarbitone (60 mg/kg, i.p.) and placed in a Kopf stereotaxic apparatus, where the head was constrained to a tilted skull position (−3.0 mm). An incision was made on the midline of the scalp and a burr hole drilled through the skull at the appropriate coordinates. Through this, an intracerebral injection was delivered into the left striatum using a 30 gauge blunt-tipped cannula. Stereotaxic coordinates for injection were: 0.3 mm anterior and 3.0 mm lateral from Bregma, and 5.2 mm ventral from the cortical surface, according to the atlas of Paxinos & Watson Lesioned rats received 4 μl of 2.5 μg/μl 6-OHDA/0.01% (w/v) ascorbic acid, while control rats received 4 μl of saline/0.01% (w/v) ascorbic acid. Injections were delivered at a rate of 0.6 μl/min and the needle left in position for 10 min following injection before being withdrawn slowly. After sealing the skull, the incision was closed and the animals allowed to recover.

Tested compounds were administered for 14 days before the induced lesions.

Protection of tested compound from loss of striatal dopaminergic nerve terminals was assessed by [³H]-mazindol autoradiography.

Seven days after stereotaxic surgery, rats were lightly anaesthetized (CO₂/O₂: 80/20) and decapitated. Brains were rapidly removed and frozen over liquid nitrogen, then stored at −40° C. prior to sectioning.

A Reichert Jung cryostat was used to cut consecutive coronal 14 μm sections of striatum at level +0.30 mm from Bregma according to the atlas of Paxinos & Watson. Sections were thaw mounted onto poly-L-lysine coated slides, then stored at −20° C. until use.

[³H]-mazindol autoradiography was used to visualize dopaminergic nerve terminals within sections of striatum taken from rat brains. All autoradiographic steps were carried out at 4° C. to reduce non-specific binding.

Slide mounted sections of striatum were preincubated for 15 min in 50 mm Tris-HCl solution (pH 7.9) containing 120 mM NaCl and 5 mM KCl. Sections were then incubated for 60 min with 4 nM [³H]-mazindol in 50 mM Tris-HCl solution (pH 7.9) containing 300 mM NaCl and 5 mM KCl. Desipramine (DMI; 300 nM) was included in all incubation solutions to prevent non-selective [³H]-mazindol binding at noradrenergic uptake sites. Nomifensine (100 μM), a selective inhibitor of dopamine uptake sites, was used to determine non-specific binding. Sections were washed twice (2×3 min) in ice-cold incubation buffer to remove excess [³H]-mazindol and dried under a stream of cold, dry air.

Once dry, radiolabelled sections were apposed to Hyperfilm-³H and exposed for 21 days to allow an image of striatal dopaminergic nerve terminal density to develop on the film. Following the exposure period, films were developed for 5 min in Phenisol X-ray developer, rinsed briefly in a weak solution of stopbath and fixed in Hypam X-ray fixer for 10 min.

Computer-assisted densitometry was used to quantify the optical density of film images. The system was calibrated using [³H]-standards, so that optical density measurements were made in nCi mm⁻². Specific binding was determined by subtracting the non-specific binding image from that of total binding, and was measured in the entire striatum.

### Data Analysis

The mean optical density and standard error of the mean were determined from independent measurements taken in at least three consecutive coronal sections of striatum for each animal.

1. A compound of Formula I, 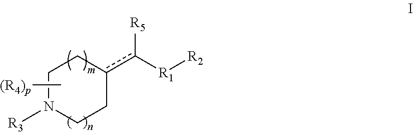
wherein:
R₁ is an optionally substituted mono-, bi- or tricyclic C₁-C₁₃ heterocyclic group containing from 1 to 5 heteroatoms selected from N, O, and S;
R₂ is hydrogen, an optionally substituted monocyclic aromatic, or a C₁-C₅ heteroaromatic group containing from 1 to 4 heteroatoms selected from N, O, and S;
R₃ is an optionally substituted mono-, bi- or tricyclic C₁-C₁₃ heterocyclic group containing 1 to 5 heteroatoms selected from N, O, and S; an optionally substituted mono-, bi- or tricyclic C₆-C₁₄ aryl group, an optionally substituted C₃-C₆ cycloalkyl group, or an optionally substituted C₃-C₆ cycloalkenyl group;
R₄ is selected independently for each position capable of substitution from the group consisting of hydrogen and C₁-C₆ alkyl;
R₅ is hydrogen, halogen, or C₁-C₆ alkyl;
m is 0, 1 or 2
n is 0, 1 or 2
p is 0, 1, 2, 3, 4, 5, or 6; and [symbol]
is an optional double bond;
wherein the optional substitutents are independently selected from halogen, oxo, nitro, cyano, hydroxy, carbamoyl, C₁-C₆ alkylsulphonyl, C₁-C₆ alkylthio, C₁-C₆ alkylcarbonyl or C₁-C₆ alkylcarbonyl-(C₁-C₆) alkyl group, or a group of the formula —NR*R* wherein each R* independently represents hydrogen, C₁-C₆ alkyl, C₁-C₆ alkylcarbonyl, phenyl and benzyl; and
C₁-C₆ alkyl, C₂-C₆ alkenyl, C₂-C₆ alkenyl or C₁-C₆ alkoxy group, each of which may be optionally bear from 1 to 8 substituents independently selected from oxo, halo, cyano, nitro, amino, hydroxy and phenyl; and
a C₃-C₉ mono- or bicycloalkyl group each of which may be optionally bear from 1 to 3 substituents independently selected from C₁-C₆ alkyl, oxo, halo, cyano, nitro, amino, hydroxy and phenyl substituents; and
a group of the formula -A, —O-A, —C(O)-A, —(CH₂)q-A, —NR**-A, —C(O)NR**-A, —NR**C(O)-A or —OC(O)-A, wherein
A is a phenyl group or a C₁-C₈ heterocyclic group containing from 1 to 3 heteroatoms selected from N, O, and S; each group A of being optionally substituted with from 1 to 3 groups independently selected from halo, hydroxy, cyano, nitro and C₁-C₆ alkyl;
R** is independently selected from hydrogen and C₁-C₆ alkyl, and
q is 0 or an integer from 1 to 6; and
enantiomers, diastereomers and N-oxides thereof and pharmaceutically acceptable salts thereof.

2. (canceled)

3. (canceled)

4. The compound of claim 1, wherein R₁ is hydrogen or an optionally substituted mono-, bi- or tricyclic C₁-C₉ heterocyclic group containing 1 to 5 heteroatoms selected from N, O, and S.

5. The compound of claim 4, wherein R₁ is an optionally substituted five membered heteroaromatic ring.

6. The compound of claim 1, wherein R₁ is an optionally substituted group selected from pyrazolyl, benzofuranyl, triazolyl, benzothiazolyl, tetrazolyl, benzoxazolyl, thiazolyl, oxazolyl, oxadiazolyl, isoxazolyl, benzimidalolyl, benzothienyl, thienobenzothienyl, and indolyl, and the optional substituent is selected from halogen, alkyl, alkoxy, nitro, amino, hydroxyl, carboxyl, cyano, and trifluoromethyl.

7. (canceled)

8. The compound of claim 1, wherein R₂ is hydrogen or an optionally substituted monocyclic aromatic or C₁-C₅ heteroaromatic group containing from 1 to 4 heteroatoms selected from the group consisting of N, S, and O.

9. The compound of claim 8, wherein R₂ is optionally substituted phenyl.

10. (canceled)

11. The compound of claim 1, wherein R₂ is an optionally substituted group selected from phenyl, furanyl, thienyl, isoxazolyl, and pyridinyl, and the optional substituent is selected from halogen, alkyl, alkoxy, nitro, amino, hydroxyl, carboxyl, cyano, and trifluoromethyl.

12. (canceled)

13. The compound of claim 1, wherein R₃ is an optionally substituted C₁-C₆ alkyl group, an optionally substituted mono-, bi- or tricyclic C₁-C₁₃ heterocyclic group containing 1 to 5 heteroatoms selected from the group consisting of N, S, and O; an optionally substituted mono, bi or tricyclic C₆-C₁₄ aryl group, an optionally substituted C₃-C₆ cycloalkyl group, or an optionally substituted C₃-C₆ cycloalkenyl group.

14. (canceled)

15. The compound of claim 13, wherein R₃ is a mono- or bicyclic C₁-C₉ heterocyclic group containing 1 to 3 heteroatoms selected from the group consisting of N, S, and O, and at least 2 adjacent carbon atoms, one carbon of which is bound to the nitrogen atom of the piperidinyl ring represented in Formula I, and the other carbon of which bears a cyano or nitro substituent.

16. The compound of claim 13, wherein R₃ is an optionally substituted group selected from phenyl, pyridyl, pyrazinyl, and thienyl, and the optional substituent is selected from halogen, alkyl, alkoxy, nitro, amino, hydroxyl, carboxyl, cyano, and trifluoromethyl.

17. (canceled)

18. The compound of claim 13, wherein R₃ is a pyridyl or phenyl group substituted with an electron withdrawing group.

19. The compound of claim 13, wherein R₃ is 6-methyl-3-nitro-2-pyridyl, 6-methyl-3-cyano-2-pyridyl, 4-methoxy-3-cyano-2-pyridyl, 3-cyano-2-thienyl, or 3-cyano-2-pyrazinyl.

20. The compound of claim 1, wherein R₁ is an optionally substituted group selected from pyrazolyl, benzofuranyl, triazolyl, benzothiazolyl, tetrazolyl, benzoxazolyl, thiazolyl, oxazolyl, oxadiazolyl, isoxazolyl, benzimidalolyl, benzothienyl, thienobenzothienyl, and indolyl, and the optional substituent is selected from halogen, alkyl, alkoxy, nitro, amino, hydroxyl, carboxyl, cyano, and trifluoromethyl;
R₂ is an optionally substituted group selected from phenyl, furanyl, thienyl, isoxazolyl, and pyridinyl, and the optional substituent is selected from halogen, alkyl, alkoxy, nitro, amino, hydroxyl, carboxyl, cyano, and trifluoromethyl;
R₃ is an optionally substituted group selected from phenyl, pyridyl, pyrazinyl, and thienyl, and the optional substituent is selected from halogen, alkyl, alkoxy, nitro, amino, hydroxyl, carboxyl, cyano, and trifluoromethyl; and
R₅ is hydrogen.

21. The compound of claim 1 selected from the group consisting of
2-(4-{[5-(2-Furyl)-1-methyl-1H-pyrazol-3-yl]methylene}piperidin-1-yl)-6-methyl-3-nitropyridine;
6-Methyl-2-{4-[(1-methyl-5-thien-2-yl-1H-pyrazol-3-yl)methylene]piperidin-1-yl}-3-nitropyridine;
2-{4-[(5-Fluoro-1-benzofuran-2-yl)methylene]piperidin-1-yl}-3-nitropyridine;
6-Methyl-2-{4-[(5-thien-2-yl-1H-pyrazol-3-yl)methylene]piperidin-1-yl}-3-nitropyridine;
3-Nitro-2-{4-[(1-phenyl-1H-1,2,3-triazol-4-yl)methylene]piperidin-1-yl}pyridine;
2-{4-[(6-Fluoro-1-benzofuran-2-yl)methylene]piperidin-1-yl}-3-nitropyridine;
2-{[1-(6-Methyl-3-nitropyridin-2-yl)piperidin-4-ylidene]methyl}-1,3-benzothiazole;
6-Methyl-3-nitro-2-{4-[(2-phenyl-2H-tetrazol-5-yl)methylene]piperidin-1-yl}pyridine;
2-{4-[(1-Benzofuran-5-phenyl-2-yl)methylene]piperidin-1-yl}-3-nitropyridine;
2-{4-[(5-Chloro-1-benzofuran-2-yl)methylene]piperidin-1-yl}-3-nitropyridine;
2-{[1-(6-Methyl-3-nitropyridin-2-yl)piperidin-4-ylidene]methyl}-1,3-benzoxazole;
3-Nitro-2-{4-[(2-phenyl-2H-tetrazol-5-yl)methylene]piperidin-1-yl}pyridine;
6-Methyl-2-{4-[(2-methyl-1,3-thiazol-4-yl)methylene]piperidin-1-yl}-3-nitropyridine;
6-Methyl-3-nitro-2-{4-[(1-phenyl-1H-1,2,3-triazol-4-yl)methylene]piperidin-1-yl}pyridine;
tert-Butyl 4-(3-trimethylsilylprop-2-ynylidene)piperidin-1-carboxyate;
6-Methyl-3-nitro-2-{4-[(5-phenylisoxazol-3-yl)methylene]piperidin-1-yl}pyridine;
2-{4-[1-Benzofuran-5-methyl-2-yl)methylene]piperidin-1-yl}-3-nitropyridine;
2-{4-[1-Benzofuran-2-yl)methylene]piperidin-1-yl}-3-nitropyridine;
6-Methyl-3-nitro-2-{4-[(5-phenyl-1,2,4-oxadiazol-3-yl)methylene]piperidin-1-yl}pyridine;
6-Methyl-3-nitro-2-{4-[(3-phenylisoxazol-5-yl)methylene]piperidin-1-yl}pyridine;
6-Methyl-3-nitro-2-{4-[(5-phenyl-1,3-oxazol-2-yl)methylene]piperidin-1-yl}pyridine;
6-Methyl-3-nitro-2-{4-[(5-thien-2-yl-1,3,4-oxadiazol-2-yl)methylene]piperidin-1-yl}pyridine;
6-Methyl-3-nitro-2-{4-[(3-thien-2-yl-1,2,4-oxadiazol-5-yl)methylene]piperidin-1-yl}pyridine;
6-Methyl-3-nitro-2-{4-[(1-phenyl-1H-1,2,3-triazol-5-yl)methylene]piperidin-1-yl}pyridine;
2-[4-(1-Benzofuran-2-ylmethylene)piperidin-1-yl]-6-methyl-3-nitropyridine;
6-Methyl-3-nitro-2-{4-[(5-phenyl-1,3,4-oxadiazol-2-yl)methylene]piperidin-1-yl}pyridine;
6-Methyl-3-nitro-2-{4-[(5-thien-2-ylisoxazol-3-yl)methylene]piperidin-1-yl}pyridine;
6-Methyl-3-nitro-2-{4-[(2-thien-2-yl-1,3-oxazol-4-yl)methylene]piperidin-1-yl}pyridine;
6-methyl-3-nitro-2-{4-[(5-(3-methoxyphenyl)-1,2,4-oxadiazol-3-yl)methylene]piperidin-1-yl}pyridine;
6-Methyl-3-nitro-2-{4-[(5-thien-2-yl-1,2,4-oxadiazol-3-yl)methylene]piperidin-1-yl}pyridine;
2-{[1-(6-Methyl-3-nitropyridin-2-yl)piperidin-4-ylidene]methyl}-1-benzofuran-4-ol;
2-(4-{[5-(2-Furyl)-1,2,4-oxadiazol-3-yl]methylene}piperidin-1-yl)-6-methyl-3-nitropyridine;
2-(4-{[5-(3-Thienyl)-1,2,4-oxadiazol-3-yl]methylene}piperidin-1-yl)-6-methyl-3-nitropyridine;
2-{[1-(6-Methyl-3-nitropyridin-2-yl)piperidin-4-ylidene]methyl}-1H-benzimidazole;
2-[4-(1-Benzothien-2-ylmethylene)piperidin-1-yl]-6-methyl-3-nitropyridine;
(2-{[1-(6-Methyl-3-nitropyridin-2-yl)piperidin-4-ylidene]methyl}-1-benzofuran-6-yl)methanol;
6-Methyl-3-nitro-2-[4-({5-[3-(trifluoromethyl)phenyl]-1,2,4-oxadiazol-3-yl}methylene)piperidin-1-yl]pyridine;
6-Methyl-2-{4-[(3-methyl-1,2,4-oxadiazol-5-yl)methylene]piperidin-1-yl}-3-nitropyridine;
6-{[1-(6-Methyl-3-nitropyridin-2-yl)piperidin-4-ylidene]methyl}-1,3-benzothiazole;
6-Methyl-3-nitro-2-{4-[1-(5-phenyl-1,2,4-oxadiazol-3-yl)ethylidene]piperidinyl}pyridine;
6-Methyl-2-(4-{[5-(3-methylphenyl)-1,2,4-oxadiazol-3-yl]methylene}piperidin-1-yl)-3-nitropyridine;
2-(4-{[5-(3,5-Dimethylisoxazol-4-yl)-1,2,4-oxadiazol-3-yl]methylene}piperidin-1-yl)-6-methyl-3-nitropyridine;
6-(1-Methylethyl)-2-{4-[(3-methyl-1,2,4-oxadiazol-5-yl)methylene]piperidin-1-yl}-3-nitropyridine;
3-Nitro-2-{4-[(5-phenyl-1,2,4-oxadiazol-3-yl)methylene]piperidin-1-yl}pyridine;
2-(4-{[5-(3-Chlorophenyl)-1,2,4-oxadiazol-3-yl]methylene}piperidin-1-yl)-6-methyl-3-nitropyridine;
3-{4-[(5-Phenyl-1,2,4-oxadiazol-3-yl)methylene]piperidin-1-yl}pyrazine-2-carbonitrile;
4-Methoxy-2-{4-[(5-phenyl-1,2,4-oxadiazol-3-yl)methylene]piperidin-1-yl}nicotinonitrile;
6-Methyl-3-nitro-2-{4-[(3-phenyl-1,2,4-oxadiazol-5-yl)methylene]piperidin-1-yl}pyridine;
2-{4-[(5-Phenyl-1,2,4-oxadiazol-3-yl)methylene]piperidin-1-yl}thiophene-3-carbonitrile;
1-Methyl-2-{[1-(6-methyl-3-nitropyridin-2-yl)piperidin-4-ylidene]methyl}-1H-benzimidazole;
2-[4-(1-Benzofuran-2-ylmethylene)piperidin-1-yl]-6-methoxy-3-nitropyridinebenzimidazole;
2-(4-{[5-(3-Fluorophenyl)-1,2,4-oxadiazol-3-yl]methylene}piperidin-1-yl)-6-methyl-3-nitropyridine;
6-Methyl-2-{4-[(3-methylisoxazol-5-yl)methylene]piperidin-1-yl}-3-nitropyridine;
6-Methyl-2-{4-[(5-methyl-1,3,4-oxadiazol-2-yl)methylene]piperidin-1-yl}-3-nitropyridine;
-Methyl-3-nitro-2-{4-[(5-phenyl-2H-tetrazol-2-yl)methylene]piperidin-1-yl}pyridine;
6-Methyl-2-(4-{[5-(6-methylpyridin-2-yl)-1,2,4-oxadiazol-3-yl]methylene}piperidin-1-yl)-3-nitropyridine;
3-{4-[(5-Thien-3-yl-1,2,4-oxadiazol-3-yl)methylene]piperidin-1-yl}pyrazine-2-carbonitrile;
2-(4-{[2-(2-Furyl)-1,3-thiazol-4-yl]methylene}piperidin-1-yl)-6-methyl-3-nitropyridine;
6-Methyl-3-nitro-2-{4-[(2-phenyl-1,3-thiazol-4-yl)methylene]piperidin-1-yl}pyridine;
2-{4-[(4-Fluoro-1-benzofuran-2-yl)methylene]piperidin-1-yl}-6-methyl-3-nitropyridine;
3-[4-(1-Benzofuran-2-ylmethylene)piperidin-1-yl]pyrazine-2-carbonitrile;
6-Methyl-3-nitro-2-{3-[(5-phenyl-1,2,4-oxadiazol-3-yl)methylene]azetidin-1-yl}pyridine;
-1-(6-Methyl-3-nitropyridin-2-yl)-4-[(5-phenyl-1,2,4-oxadiazol-3-yl)methylene]azepane;
6-Methyl-2-(4-{[5-(3-methylphenyl)-2H-tetrazol-2-yl]methylene}piperidin-1-yl)-3-nitropyridine;
6-Methyl-3-nitro-2-{(3Z)-3-[(5-phenyl-1,2,4-oxadiazol-3-yl)methylene]pyrrolidin-1-yl}pyridine;
6-Methyl-3-nitro-2-{(3E)-3-[(5-phenyl-1,2,4-oxadiazol-3-yl)methylene]pyrrolidin-1-yl}pyridine;
6-Methyl-3-nitro-2-[4-(thieno[3,2-b][1]benzothien-2-ylmethylene)piperidin-1-yl]pyridine;
2-{4-[(7-Fluoro-1-benzofuran-2-yl)methylene]piperidin-1-yl}-6-methyl-3-nitropyridine;
tert-Butyl 6-methoxy-2-[[1-(6-methyl-3-nitro-2-pyridyl)-4-piperidylidene]methyl]indole-1-carboxylate;
6-Fluoro-2-{[1-(6-methyl-3-nitropyridin-2-yl)piperidin-4-ylidene]methyl}-1H-indole;
2-(4-{[3-(3-Chlorophenyl)-1,2,4-oxadiazol-5-yl]methylene}piperidin-1-yl)-6-methyl-3-nitropyridine;
6-Methyl-3-nitro-2-{4-[(5-pyridin-4-yl-1,2,4-oxadiazol-3-yl)methylene]piperidin-1-yl}pyridine;
6-Methyl-3-nitro-2-{4-[(5-pyridin-4-yl-1,2,4-oxadiazol-3-yl)methylene]piperidin-2-yl}pyridine; and
6-Methyl-3-nitro-2-{4-[(5-pyridin-4-yl-1,2,4-oxadiazol-3-yl)methylene]piperidin-3-yl}pyridine.

22. A compound of claim 1, wherein the compound is represented by Formula II 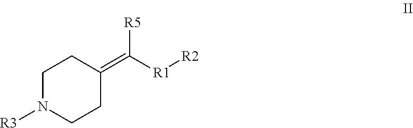
wherein
R₁ is an optionally substituted mono-, bi- or tricyclic C₁-C₁₃ heterocyclic group containing from 1 to 5 heteroatoms selected from N, O, and S;
R₂ is hydrogen, an optionally substituted monocyclic aromatic, or a C₁-C₅ heteroaromatic group containing from 1 to 4 heteroatoms selected from N, O, and S;
R₃ is an optionally substituted mono-, bi- or tricyclic C₁-C₁₃ heterocyclic group containing 1 to 5 heteroatoms selected from N, O, and S; an optionally substituted mono, bi or tricyclic C₆-C₁₄ aryl group, an optionally substituted C₃-C₆ cycloalkyl group, or an optionally substituted C₃-C₆ cycloalkenyl group;
R₄ is selected independently for each position capable of substitution from the group consisting of hydrogen and C₁-C₆ alkyl;
R₅ is hydrogen, halogen, or C₁-C₆ alkyl; and
enantiomers, diastereomers and N-oxides thereof and pharmaceutically acceptable salts thereof.

23. The compound of claim 22, wherein
R₁ is an optionally substituted group selected from pyrazolyl, benzofuranyl, triazolyl, benzothiazolyl, tetrazolyl, benzoxazolyl, thiazolyl, oxazolyl, oxadiazolyl, isoxazolyl, benzimidalolyl, benzothienyl, thienobenzothienyl, and indolyl;
R₂ is an optionally substituted group selected from phenyl, furanyl, thienyl, isoxazolyl, and pyridinyl.
R₃ is an optionally substituted group selected from phenyl, pyridyl, pyrazinyl, and thienyl; and
R₅ is hydrogen or methyl.

24. The compound of claim 23, wherein the optional substituents for R₁, R₂, and R₃ are selected from halogen, alkyl, alkoxy, nitro, amino, hydroxyl, carboxyl, cyano, and trifluoromethyl.

25. A pharmaceutical composition comprising a pharmaceutically acceptable excipient or diluent and a therapeutically effective amount of a compound according to claim 1 or a pharmaceutically acceptable salt thereof.

26. A method of treating neuromuscular dysfunctions of the lower urinary tract comprising administering to a mammal in need of such treatment an effective amount of a compound of claim 1.

27. The method according to claim 26, wherein said neuromuscular dysfunction is selected from the group consisting of urinary urgency, overactive bladder, increased urinary frequency, decreased urinary compliance (decreased bladder storage capacity), cystitis, interstitial cystitis, incontinence, urine leakage, enuresis, dysuria, urinary hesitancy and difficulty in emptying the bladder.

28. The method according to claim 26, wherein said compound is administered in combination with an antimuscarinic drug, an α1-adrenergic antagonist, a serotonin and/or noradrenaline reuptake inhibitor, or a selective or non-selective COX inhibitor.

29. The method according to claim 28, wherein said antimuscarinic drug is selected from the group consisting of oxybuynin, tolterodine, darifenacin, solifenacin, trospium, imidafenacin, fesoterodine and temiverine, said α1-adrenergic antagonist is selected from the group consisting of prazosin, doxazosin, terazosin, alfuzosin, silodosin and tamsulosin; said serotonin and/or noradrenaline reuptake inhibitor is selected form the group consisting of duloxetine, milnacipran, amoxapine, venlafaxine, des-venlafaxine, sibutramine, tesofensine and des-methylsibutramine, and said selective or non-selective COX inhibitor is selected from the group consisting of ibuprofen, naproxen, benoxaprofen, flurbiprofen, fenoprfen, ketoprofen, indoprofen, pirprofen, carprofen, tioxaprofe, suprofen, tiaprofenic acid, fluprofen, indomethacin, sulindac, tolmetin, zomepirac, diclofenac, fenclofenac, ibufenac, acetyl salicylic acid, piroxicam, tenoxicam, nabumetone, ketorolac, azapropazone, mefenamic acid, tolfenamic acid, diflunisal, acemetacin, fentiazac, clidanac, meclofenamic acid, flufenamic acid, niflumic acid, flufenisal, sudoxicam, etodolac, salicylic acid, benorylate, isoxicam, 2-fluoro-α-methyl[1,1′-biphenyl]-4-acetic acid 4-(nitrooxy)butyl ester, meloxicam, parecoxib and nimesulide.

30-35. (canceled)

36. The method according to claim 26 wherein said mammal is a human.

37. The method of claim 26, wherein the compound of Formula I is represented by Formula II 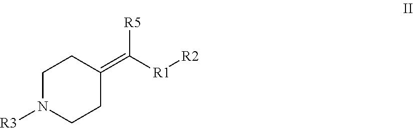
wherein
R₁ is an optionally substituted group selected from pyrazolyl, benzofuranyl, triazolyl, benzothiazolyl, tetrazolyl, benzoxazolyl, thiazolyl, oxazolyl, oxadiazolyl, isoxazolyl, benzimidalolyl, benzothienyl, thienobenzothienyl, and indolyl;
R₂ is an optionally substituted group selected from phenyl, furanyl, thienyl, isoxazolyl, and pyridinyl.
R₃ is an optionally substituted group selected from phenyl, pyridyl, pyrazinyl, and thienyl; and
R₅ is hydrogen or methyl;
the optional substituents for R₁, R₂, and R₃ are selected from halogen, alkyl, alkoxy, nitro, amino, hydroxyl, carboxyl, cyano, and trifluoromethyl; and
enantiomers, diastereomers and N-oxides thereof and pharmaceutically acceptable salts thereof.

38-39. (canceled)

40. A method of treating a disease or disorder selected from migraine, GERD, anxiety disorder, abuse disorder, substance dependence disorder, substance withdrawal disorder, neuropathic pain disorder, and fragile X syndrome disorder comprising administering to a mammal in need of such treatment an effective amount of a compound of claim 1.

41. The method according to claim 40 wherein said mammal is a human.

42. The method of claim 40, wherein the compound of Formula I is represented by Formula II 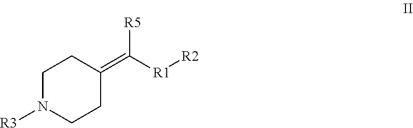
wherein
R₁ is an optionally substituted group selected from pyrazolyl, benzofuranyl, triazolyl, benzothiazolyl, tetrazolyl, benzoxazolyl, thiazolyl, oxazolyl, oxadiazolyl, isoxazolyl, benzimidalolyl, benzothienyl, thienobenzothienyl, and indolyl;
R₂ is an optionally substituted group selected from phenyl, furanyl, thienyl, isoxazolyl, and pyridinyl.
R₃ is an optionally substituted group selected from phenyl, pyridyl, pyrazinyl, and thienyl; and
R₅ is hydrogen or methyl;
the optional substituents for R₁, R₂, and R₃ are selected from halogen, alkyl, alkoxy, nitro, amino, hydroxyl, carboxyl, cyano, and trifluoromethyl; and
enantiomers, diastereomers and N-oxides thereof and pharmaceutically acceptable salts thereof.

43-44. (canceled)

45. A The method of claim 40 wherein the disease or disorder is GERD.

46-49. (canceled)

50. The method of claim 40 wherein the disease or disorder is anxiety disorder.

51-54. (canceled)

55. The method of claim 40 wherein the disease or disorder is abuse disorder, substance dependence disorder or substance withdrawal disorder.

56-59. (canceled)

60. The method of claim 40 wherein the disease or disorder is neuropathic pain disorder.

61-64. (canceled)

65. The method of claim 40 wherein the disease or disorder is fragile X syndrome disorders.

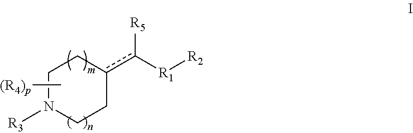

66-69. (canceled)

70. A compound having the general formula I 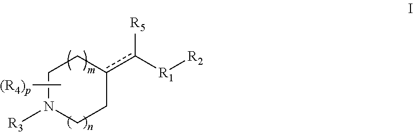
wherein:
R₁ represents an optionally substituted C₁-C₁₃ heteromonocyclic, heterobicyclic or heterotricyclic group containing from 1 to 5 heteroatoms selected from nitrogen, oxygen and sulphur;
R₂ represents a hydrogen atom, an optionally substituted monocyclic aromatic group, or a C₁-C₅ heteroaromatic group containing from 1 to 4 heteroatoms selected from nitrogen, oxygen and sulphur;
R₃ represents an optionally substituted C₁-C₁₃ heteromonocyclic, heterobicyclic or heterotricyclic group containing from 1 to 5 heteroatoms selected from nitrogen, oxygen and sulphur; an optionally substituted mono-, bi- or tricyclic C₆-C₁₄ aryl group, an optionally substituted C₃-C₆ cycloalkyl group, or an optionally substituted C₃-C₆ cycloalkenyl group;
each R₄, independently for each position capable of substitution, represents a hydrogen atom or a C₁-C₆ alkyl group;
R₅ represents a hydrogen or halogen atom or a C₁-C₆ alkyl group;
m is 0, 1 or 2;
n is 0, 1 or 2;
p is 0, 1, 2, 3, 4, 5, or 6; and [symbol]
is an optional double bond;
the optional substituents for each optionally substituted group being selected from halogen atoms and/or from nitro, cyano, hydroxy, oxo, carbamoyl, (C₁-C₆)-alkyl, (C₂-C₆)-alkenyl, (C₂-C₆)-alkynyl, (C₁-C₆)-alkoxy, (C₁-C₆)-alkylsulphonyl, (C₁-C₆)-alkylthio, (C₁-C₆)-alkylcarbonyl, hydroxy-(C₁-C₆)-alkyl, halo-(C₁-C₆)-alkyl, di-(C₁-C₆)-alkylaminocarbonyl, (C₁-C₆)-alkylcarbonyl-(C₁-C₆)-alkyl and (C₁-C₆)-alkoxycarbonyl groups and/or from groups of the formula —NR*R* wherein each R* independently represents a hydrogen atom or a (C₁-C₆)-alkyl, (C₁-C₆)-alkylcarbonyl, phenyl or benzyl group;
or an enantiomer, diastereomer, N-oxide or pharmaceutically acceptable salt of such a compound.

71. A compound according to claim 70 wherein R₁ represents an optionally substituted C₁-C₉ heteromonocyclic or heterobicyclic group containing from 1 to 4 heteroatoms selected from nitrogen, oxygen and sulphur.

72. (canceled)

73. A compound according to claim 71 wherein R₁ represents an optionally substituted isoxazolyl, oxazolyl, oxadiazolyl, pyrazolyl, tetrazolyl, thiazolyl or triazolyl, benzimidazolyl, benzofuranyl, benzoxazolyl, benzothiazolyl, benzothienyl or indolyl group, wherein
each optional substituent is a halogen atom or an alkyl, alkoxy, hydroxy, hydroxyalkyl or alkoxycarbonyl group.

74-76. (canceled)

77. A compound according to claim 70 wherein R₁ represents a 1,2,3-triazol-4-yl, 1,2,3-triazol-5-yl, 1,2,4-oxadiazol-3-yl, 3-isopropyl-1,2,4-oxadiazol-5-yl, 3-methyl-1,2,4-oxadiazol-5-yl, 1,2,4-oxadiazol-5-yl, 5-methyl-1,3,4-oxadiazol-2-yl, 1,3,4-oxadiazol-2-yl, 1,3-benzothiazol-2-yl, 1,3-benzothiazol-6-yl, 1,3-benzoxazol-2-yl, 1,3-oxazol-2-yl, 1,3-oxazol-4-yl, 2-methyl-1,3-thiazol-4-yl, 1,3-thiazol-4-yl, 4-fluoro-1-benzofuran-2-yl, 4-hydroxy-1-benzofuran-2-yl, 5-chloro-1-benzofuran-2-yl, 5-fluoro-1-benzofuran-2-yl, 5-methyl-1-benzofuran-2-yl, 6-fluoro-1-benzofuran-2-yl, 6-hydroxymethyl-1-benzofuran-2-yl, 7-fluoro-1-benzofuran-2-yl, 1-benzofuran-2-yl, 1-benzothien-2-yl, 1-methyl-3-pyrazolyl, 3-pyrazolyl, 1-methyl-benzimidazol-2-yl, benzimidazol-2-yl, 1-t.butoxycarbonyl-6-methoxy-indol-2-yl, 6-fluoro-indol-2-yl, isoxazol-3-yl, 3-methyl-isoxazol-5-yl, isoxazol-5-yl, tetrazol-2-yl, tetrazol-5-yl or thieno[3,2-b][1]benzothien-2-yl group.

78. A compound according to claim 70 wherein R₂ represents a hydrogen atom, an optionally substituted phenyl group or an optionally substituted C₁-C₅ heteroaromatic group containing from 1 to 4 heteroatoms selected from nitrogen, oxygen and sulphur.

79. A compound according to claim 70 wherein R₂ represents a hydrogen atom, an optionally substituted phenyl group or an optionally substituted furyl, isoxazolyl, pyridyl or thienyl group wherein
each optional substituent is a halogen atom, an alkyl group, an alkoxy group or a trifluoromethyl group.

80. (canceled)

81. A compound according to claim 78 wherein R₂ represents a hydrogen atom or a phenyl, 3-chlorophenyl, 3-fluorophenyl, 3-methoxyphenyl, 3-methylphenyl, 3-trifluoromethylphenyl, 2-furyl, 3,5-dimethyl-isoxazol-4-yl, 2-pyridyl, 6-methyl-2-pyridyl, 3-pyridyl, 4-pyridyl, 2-thienyl or 3-thienyl group.

82. A compound according to claim 70 wherein R₃ represents an optionally substituted C₂-C₉ heteromonocyclic or heterobicyclic group containing from 1 to 3 heteroatoms selected from nitrogen, oxygen and sulphur, and at least 2 adjacent carbon atoms, one of which is bonded to the nitrogen atom of the nitrogen containing ring in the general formula I, and the other of which bears a cyano or nitro substituent.

83. A compound according to claim 82 wherein R₃ represents a 2-cyano-3-pyrazinyl, 3-cyano-2-thienyl, 3-cyano-4-methoxy-2-pyridyl, 3-cyano-6-methyl-2-pyridyl, 3-nitro-2-pyridyl, 6-methoxy-3-nitro-2-pyridyl or 6-methyl-3-nitro-2-pyridyl group.

84. A compound according to claim 70 wherein m=n=1 and p=0.

85. (canceled)

86. Any one of the following compounds according to claim 70:
1-(3-nitro-2-pyridyl)-4-[(5-fluoro-1-benzofuran-2-yl)-methylene]-piperidine,
1-(6-methyl-3-nitro-2-pyridyl)-4-{[1-methyl-5-(2-thienyl)-1H-pyrazol-3-yl]-methylene}-piperidine,
1-(3-nitro-2-pyridyl)-4-[(5-fluoro-1-benzofuran-2-yl)-methylene]-piperidine,
1-(6-methyl-3-nitro-2-pyridyl)-4-{[5-(2-thienyl)-1H-pyrazol-3-yl]-methylene}-piperidine,
1-(3-nitro-2-pyridyl)-4-[(1-phenyl-1H-1,2,3-triazol-4-yl)-methylene]-piperidine,
1-(3-nitro-2-pyridyl)-4-[(6-fluoro-1-benzofuran-2-yl)-methylene]-piperidine,
1-(6-methyl-3-nitro-2-pyridyl)-4-[(1,3-benzothiazol-2-yl)-methylene]-piperidine,
1-(6-methyl-3-nitro-2-pyridyl)-4-[(2-phenyl-2H-tetrazol-5-yl)-methylene]-piperidine,
1-(3-nitro-2-pyridyl)-4-[(5-phenyl-1-benzofuran-2-yl)-methylene]-piperidine,
1-(3-nitro-2-pyridyl)-4-[(5-chloro-1-benzofuran-2-yl)-methylene]-piperidine,
1-(6-methyl-3-nitro-2-pyridyl)-4-[(1,3-benzoxazol-2-yl)-methylene]-piperidine,
1-(3-nitro-2-pyridyl)-4-[(2-phenyl-2H-tetrazol-5-yl)-methylene]-piperidine,
1-(6-methyl-3-nitro-2-pyridyl)-4-[(2-methyl-1,3-thiazol-4-yl)-methylene]-piperidine,
1-(6-methyl-3-nitro-2-pyridyl)-4-[(1-phenyl-1H-1,2,3-triazol-4-yl)-methylene]-piperidine,
1-(6-methyl-3-nitro-2-pyridyl)-4-[(5-phenyl-isoxazol-3-yl)-methylene]-piperidine,
1-(3-nitro-2-pyridyl)-4-[(5-methyl-1-benzofuran-2-yl)-methylene]-piperidine,
1-(3-nitro-2-pyridyl)-4-[(1-benzofuran-2-yl)-methylene]-piperidine,
1-(6-methyl-3-nitro-2-pyridyl)-4-[(5-phenyl-1,2,4-oxadiazol-3-yl)-methylene]-piperidine,
1-(6-methyl-3-nitro-2-pyridyl)-4-[(3-phenyl-isoxazol-5-yl)-methylene]-piperidine,
1-(6-methyl-3-nitro-2-pyridyl)-4-[(5-phenyl-1,3-oxazol-2-yl)-methylene]-piperidine,
1-(6-methyl-3-nitro-2-pyridyl)-4-{[5-(2-thienyl)-1,3,4-oxadiazol-2-yl]-methylene}-piperidine,
1-(6-methyl-3-nitro-2-pyridyl)-4-{[3-(2-thienyl)-1,2,4-oxadiazol-5-yl]-methylene}-piperidine,
1-(6-methyl-3-nitro-2-pyridyl)-4-[(1-phenyl-1H-1,2,3-triazol-5-yl)-methylene]-piperidine,
1-(6-methyl-3-nitro-2-pyridyl)-4-[(1-benzofuran-2-yl)-methylene]-piperidine,
1-(6-methyl-3-nitro-2-pyridyl)-4-[5-phenyl-1,3,4-oxadiazol-2-yl)-methylene]-piperidine,
1-(6-methyl-3-nitro-2-pyridyl)-4-{[5-(2-thienyl)-isoxazol-3-yl]-methylene}-piperidine,
1-(6-methyl-3-nitro-2-pyridyl)-4-{[2-(2-thienyl)-1,3-oxazol-4-yl]-methylene}-piperidine,
1-(6-methyl-3-nitro-2-pyridyl)-4-{[5-(3-methoxyphenyl)-1,2,4-oxadiazol-3-yl]-methylene}-piperidine,
1-(6-methyl-3-nitro-2-pyridyl)-4-{[5-(2-thienyl)-1,2,4-oxadiazol-3-yl]-methylene}-piperidine,
1-(6-methyl-3-nitro-2-pyridyl)-4-[(4-hydroxy-1-benzofuran-2-yl)-methylene]-piperidine,
1-(6-methyl-3-nitro-2-pyridyl)-4-{[5-(2-furyl)-1,2,4-oxadiazol-3-yl]-methylene}-piperidine,
1-(6-methyl-3-nitro-2-pyridyl)-4-{[5-(3-thienyl)-1,2,4-oxadiazol-3-yl]-methylene}-piperidine,
1-(6-methyl-3-nitro-2-pyridyl)-4-[(1H-benzimidazol-2-yl)-methylene]-piperidine,
1-(6-methyl-3-nitro-2-pyridyl)-4-[(1-benzothien-2-yl)-methylene]-piperidine,
1-(6-methyl-3-nitro-2-pyridyl)-4-[(6-hydroxymethyl-1-benzofuran-2-yl)-methylene]-piperidine,
1-(6-methyl-3-nitro-2-pyridyl)-4-{[5-(3-trifluoromethylphenyl)-1,2,4-oxadiazol-3-yl]-methylene}-piperidine,
1-(6-methyl-3-nitro-2-pyridyl)-4-[(3-methyl-1,2,4-oxadiazol-5-yl)-methylene]-piperidine,
1-(6-methyl-3-nitro-2-pyridyl)-4-[(1,3-benzothiazol-6-yl)-methylene]-piperidine,
1-(6-methyl-3-nitro-2-pyridyl)-4-[1-(5-phenyl-1,2,4-oxadiazol-3-yl)-ethylidene]-piperidine,
1-(6-methyl-3-nitro-2-pyridyl)-4-{[5-(3-methylphenyl)-1,2,4-oxadiazol-3-yl]-methylene}-piperidine,
1-(6-methyl-3-nitro-2-pyridyl)-4-{[5-(3,5-dimethylisoxazol-4-yl)-1,2,4-oxadiazol-3-yl]-methylene}-piperidine,
1-(6-methyl-3-nitro-2-pyridyl)-4-[(3-isopropyl-1,2,4-oxadiazol-5-yl)-methylene]-piperidine,
1-(3-nitro-2-pyridyl)-4-[(5-phenyl-1,2,4-oxadiazol-3-yl)-methylene]-piperidine,
1-(6-methyl-3-nitro-2-pyridyl)-4-{[5-(3-chlorophenyl)-1,2,4-oxadiazol-3-yl]-methylene}-piperidine,
1-(2-cyano-3-pyrazinyl)-4-[(5-phenyl-1,2,4-oxadiazol-3-yl)-methylene]-piperidine,
1-(2-cyano-4-methoxy-3-pyrazinyl)-4-[(5-phenyl-1,2,4-oxadiazol-3-yl)-methylene]-piperidine,
1-(6-methyl-3-nitro-2-pyridyl)-4-[(3-phenyl-1,2,4-oxadiazol-5-yl)-methylene]-piperidine,
1-(3-cyano-2-thienyl)-4-[(5-phenyl-1,2,4-oxadiazol-3-yl)-methylene]-piperidine,
1-(6-methyl-3-nitro-2-pyridyl)-4-[(1-methyl-1H-benzimidazol-2-yl)-methylene]-piperidine,
1-(6-methoxy-3-nitro-2-pyridyl)-4-[(1-benzofuran-2-yl)-methylene]-piperidine,
1-(6-methyl-3-nitro-2-pyridyl)-4-{[5-(3-fluorophenyl)-1,2,4-oxadiazol-3-yl]-methylene}-piperidine,
1-(6-methyl-3-nitro-2-pyridyl)-4-[(3-methyl-isoxazol-5-yl)-methylene]-piperidine,
1-(6-methyl-3-nitro-2-pyridyl)-4-[(5-methyl-1,3,4-oxadiazol-2-yl)-methylene]-piperidine,
1-(6-methyl-3-nitro-2-pyridyl)-4-[(5-phenyl-2H-tetrazol-2-yl)-methylene]-piperidine,
1-(6-methyl-3-nitro-2-pyridyl)-4-{[5-(6-methyl-2-pyridyl)-1,2,4-oxadiazol-3-yl]-methylene}-piperidine,
1-(2-cyano-3-pyrazinyl)-4-{[5-(3-thienyl)-1,2,4-oxadiazol-3-yl]-methylene}-piperidine,
1-(6-methyl-3-nitro-2-pyridyl)-4-{[(2-(2-furyl)-1,3-thiazol-4-yl]-methylene}-piperidine,
1-(6-methyl-3-nitro-2-pyridyl)-4-[(2-phenyl-1,3-thiazol-4-yl)-methylene]-piperidine,
1-(6-methyl-3-nitro-2-pyridyl)-4-[(4-fluoro-1-benzofuran-2-yl)-methylene]-piperidine,
1-(2-cyano-3-pyrazinyl)-4-[(1-benzofuran-2-yl)-methylene]-piperidine,
1-(6-methyl-3-nitro-2-pyridyl)-3-[(5-phenyl-1,2,4-oxadiazol-3-yl)-methylene]-azetidine,
1-(6-methyl-3-nitro-2-pyridyl)-(4E,Z)-4-[(5-phenyl-1,2,4-oxadiazol-3-yl)-methylene]-perhydroazepine,
1-(6-methyl-3-nitro-2-pyridyl)-4-{[5-(3-methylphenyl)-2H-tetrazol-2-yl]-methylene}-piperidine,
1-(6-methyl-3-nitro-2-pyridyl)-(3Z)-3-[(5-phenyl-1,2,4-oxadiazol-3-yl)-methylene]-pyrrolidine,
1-(6-methyl-3-nitro-2-pyridyl)-(3E)-3-[(5-phenyl-1,2,4-oxadiazol-3-yl)-methylene]-pyrrolidine,
1-(6-methyl-3-nitro-2-pyridyl)-4-(thieno[3,2-b]benzothien-2-yl)-methylene}-piperidine,
1-(6-methyl-3-nitro-2-pyridyl)-4-[(7-fluoro-1-benzofuran-2-yl)-methylene]-piperidine,
1-(6-methyl-3-nitro-2-pyridyl)-4-{[1-(t-butoxycarbonyl)-6-methoxy-2-indolyl]-methylene}-piperidine,
1-(6-methyl-3-nitro-2-pyridyl)-4-[(6-fluoro-2-indolyl)-methylene]-piperidine,
1-(6-methyl-3-nitro-2-pyridyl)-4-{[3-(3-chlorophenyl)-1,2,4-oxadiazol-5-yl]-methylene}-piperidine,
1-(6-methyl-3-nitro-2-pyridyl)-4-{[5-(4-pyridyl)-1,2,4-oxadiazol-3-yl]-methylene}-piperidine,
1-(6-methyl-3-nitro-2-pyridyl)-4-{[5-(2-pyridyl)-1,2,4-oxadiazol-3-yl]-methylene}-piperidine,
1-(6-methyl-3-nitro-2-pyridyl)-4-{[5-(3-pyridyl)-1,2,4-oxadiazol-3-yl]-methylene}-piperidine,
1-(6-methyl-3-nitro-2-pyridyl)-4-{1-[5-(3-chlorophenyl)-1,2,4-oxadiazol-3-yl]-propylidene}-piperidine,
1-(6-methyl-3-nitro-2-pyridyl)-4-{1-[5-(3-chlorophenyl)-1,2,4-oxadiazol-3-yl]-ethylidene}-piperidine, or
1-(3-cyano-6-methyl-2-pyridyl)-4-{[3-(3-chlorophenyl)-1,2,4-oxadiazol-5-yl]-methylene}-piperidine.

87. A pharmaceutical composition comprising a compound according to claim 70, in admixture with a pharmaceutically acceptable diluent or carrier.
